(19) United States
(12) Patent Application Publication
(10) Pub. No.: US 2026/0115250 A1
(43) Pub. Date: Apr. 30, 2026

(54) METHODS OF TREATING AGE-RELATED MACULAR DEGENERATION

(71) Applicant: Character Biosciences, Inc., Jersey City, NJ (US)

(72) Inventors: Jonathan GUMUCIO, San Francisco, CA (US); Erik KARRER, Los Altos, CA (US); Nicholas CHURCH, San Francisco, CA (US); Marcel VAN DER BRUG, Daly City, CA (US)

(21) Appl. No.: 19/433,697

(22) Filed: Dec. 26, 2025

Related U.S. Application Data
(63) Continuation of application No. 18/865,678.
(60) Provisional application No. 63/341,990, filed on May 13, 2022.

(51) Int. Cl. A61K 38/10

(52) U.S. Cl. CPC A61K 38/10

(57) ABSTRACT

Methods of treating age-related macular degeneration (AMD) may include the administration of amphipathic, ATP binding cassette transporter membrane agonists to reduce the amount of drusen in the subject in need thereof. These methods may be particularly useful for treatment of intermediate AMD.

### CROSS REFERENCE TO RELATED APPLICATIONS

This patent application is a continuation of U.S. patent application Ser. No. 18/865,678, entitled “THERAPEUTIC LIPID PROCESSING COMPOSITIONS AND METHODS FOR TREATING AGE-RELATED MACULAR DEGENERATION,” filed on Nov. 13, 2024, now U.S. Patent Application Publication No. 2025/0302911, which is a national phase application under 35 USC 371 of International Patent No. PCT/US2023/067012, entitled “THERAPEUTIC LIPID PROCESSING COMPOSITIONS AND METHODS FOR TREATING AGE-RELATED MACULAR DEGENERATION” filed on May 15, 2023, now International Publication No. WO 2023/220756, which claims priority to U.S. provisional patent application No. 63/341,990, entitled “THERAPEUTIC LIPID PROCESSING COMPOSITIONS AND METHODS FOR TREATING AGE-RELATED MACULAR DEGENERATION,” filed on May 13, 2022, each of which are herein incorporated by reference in its entirety.

### SEQUENCE LISTING

The instant application contains a Sequence Listing which has been filed electronically in ASCII format and is hereby incorporated by reference in its entirety. Said ASCII copy, created on May 12, 2023 is named 14814-702.US0.xml and is 164,170 bytes in size.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Age-related macular degeneration (AMD) is a chronic metabolic inflammatory disease of the eye. AMD is the leading cause of blindness in people over 55 years old and has a high prevalence in the US (e.g., 8.7%) and worldwide. Further, this problem is expected to increase as global populations age. Although AMD is categorized into a variety of types (e.g., early, intermediate, wet and dry), the majority of AMD cases are considered “Dry” AMD, for which there are no approved therapies.

There are believed to be many associated factors that may contribute to AMD. Dysregulation of lipid transport and processing has been attributed to the onset and progression of AMD. Dysregulation of lipid homeostasis may result in the accumulation of lipid deposits (drusen) throughout extracellular matrices such as Bruch's membrane. Drusen are the first pathological signs of AMD that disrupt and stress retinal pigment epithelium (RPE) cells, the loss of which leads to photoreceptor degeneration and the severe later stages of the disease, including geographic atrophy (GA) and can further lead to neovascular AMD (nvAMD).

Currently, there is no cure for AMD and treatments are generally ineffective. Statins or other potential treatments often consider the disruption of cholesterol synthesis. However, there is a failure in the current state of the art to address underlying defects involved in the pathogenesis of AMD. There is a need for therapies that may treat both dry and wet AMD.

### SUMMARY OF THE DISCLOSURE

Described herein are compositions for treatment of AMD and methods of using them. Certain compositions and methods consider cellular lipid efflux, reverse cholesterol transportation (RCT) mechanisms and trafficking of lipids from within a cell. For example, described herein are compositions including one or more small peptides (e.g., peptides of 80 amino acids or smaller, 75 amino acids or smaller, 70 amino acids or smaller, 65 amino acids or smaller, 60 amino acids or smaller, 55 amino acids or smaller, 50 amino acids or smaller, 45 amino acids or smaller, 40 amino acids or smaller, etc.) that mimic the ability of apolipoprotein to regulate lipid transport and promote cellular lipid efflux via RCT mechanisms, such as ATP binding cassette (ABC) transporters, High-Density Lipoproteins (HDL), or Scavenger Receptor Class B Type 1 (SR-B1). These peptides, which may be referred to herein as apolipoprotein peptide mimetics, therapeutic apolipoprotein peptide mimetics, test peptides, peptide candidates, or simply therapeutic peptides, may include sequences engineered for improved or comparative amphipathic helical structure and function related to endogenous forms of one or more apolipoproteins (such as ApoE, ApoA, ApoJ). For example, in some variations described herein the peptides may include a modified or partial polypeptide sequence corresponding to the sequence relating to the lipid-binding or lipid-accepting structure of the apolipoprotein.

In general, these therapeutic peptides may initiate or mediate trafficking of lipids out of cells via one or more transporter proteins involved in cholesterol efflux mechanisms (shown herein by promoting cholesterol efflux from human microglial cell lines and human retinal pigment epithelial cell lines). For example, cholesterol efflux regulatory proteins, ATP binding cassette transporters proteins (e.g., ABCA1 or ABCG1, e.g., “ATP binding cassette subfamily A member 1”), or scavenger proteins may be involved in lipid efflux for cholesterol removal via RCT mechanisms. The therapeutic peptides described herein may have ABCA1-dependent lipid efflux activity (as measured by reduction of lipid export, e.g., by addition of siRNA targeting ABCA1). These therapeutic peptides may treat, prevent, or ameliorate dysregulated cholesterol homeostasis associated with deficiencies or mutations in one or more endogenous apolipoproteins, apolipoprotein receptors or lipoprotein particle maturation factors.

Also described herein are compositions for treatment of AMD and methods of using them. Certain compositions and methods consider lipid influx or trafficking of extracellular lipids via lipid influx mechanisms. For example, described herein are compositions including one or more small peptides (e.g., peptides of 80 amino acids or smaller, 75 amino acids or smaller, 70 amino acids or smaller, 65 amino acids or smaller, 60 amino acids or smaller, 55 amino acids or smaller, 50 amino acids or smaller, 45 amino acids or smaller, 40 amino acids or smaller, etc.) that mimic the ability of apolipoprotein to solubilize lipids and transport lipids into cells via cellular lipid uptake mechanisms, such as Low Density Lipoprotein Receptor (LDLR), Scavenger Receptor Class B Type 1 (SR-B1), or glycosaminoglycan (GAG)-dependent mechanisms. These peptides, which may be referred to herein as Apolipoprotein peptide mimetics, therapeutic apolipoprotein peptide mimetics, test peptides or simply therapeutic peptides, may include sequences engineered for improved amphipathic helical properties that are different from those of native sequences from apolipoproteins (ApoE, ApoA, ApoJ). For example, in some variations described herein the peptides may include a modified or partial polypeptide sequence corresponding to helix 4 of ApoE (e.g., amino acids 140-150, see, e.g., SEQ ID NO. 8).

Thus, in general, these therapeutic peptides may bind LDLR in a lipid-dependent manner and transport lipids into cells via LDLR. These therapeutic peptides may overcome lipid transport deficiency present in ApoE2 carriers (e.g., the ApoE2 variant has defective LDLR binding activity). The therapeutic peptides described herein may have lipid-dependent LDLR binding activity in a surprisingly small polypeptide (e.g., 80 or fewer amino acids, 75 or fewer amino acids, 70 or fewer amino acids, 65 or fewer amino acids, 60 or fewer amino acids, 55 or fewer amino acids, 50 or fewer amino acids, 49 or fewer amino acids, 48 or fewer amino acids, 47 or fewer amino acids, 46 or fewer amino acids, 45 or fewer amino acids, 44 or fewer amino acids, 43 or fewer amino acids, 42 or fewer amino acids, 41 or fewer amino acids, 40 or fewer amino acids, etc.). These therapeutic polypeptides may preserve lipid-dependent LDLR binding and lipid transport activity in a smaller peptide.

In addition these therapeutic peptides may solubilize lipids (e.g., shown herein by reducing turbidity of 1,2-dimyristoyl-sn-glycero-3-phosphocholine, or DMPC, liposome solutions) and may preferentially bind to oxidized vs non-oxidized lipids (as measured by surface plasmon resonance, SPR). The therapeutic peptides described herein may import lipids into the cells (as shown by labeled cholesterol import into APRE-19 and HepG2 cells), and this lipid import may be dependent on GAG binding domains on the peptides (GAG-dependent lipid import activity, as measured by reduction of lipid import into cells by addition of exogenous heparin). The therapeutic peptides described herein may have LDLR-dependent lipid import activity (as measured by reduction of lipid import into cells, e.g., by addition of siRNA targeting LDLR). Some of the example therapeutic peptides described herein may have SR-B1-dependent lipid import activity (measured by reduction of lipid import into cells by addition of siRNA targeting SR-B1). In general, the therapeutic polypeptides described herein have minimal cytotoxicity (as measured by membrane permeable dye staining of ARPE-19 cells and hemolysis of human RBC).

Thus, the therapeutic peptides described herein may function as lipid transport peptides that mimic the behavior of apolipoproteins, lipoprotein particles, lipid exporters, and lipid importers. In general, the therapeutic peptides described herein may bind to LDLR and may increase the import of lipids into LDLR+ cells, causing improved or preventing dysregulated lipid transport, a hallmark of drusen formation and AMD disease progression. The therapeutic peptides described herein may also bind to ABCA1 and may increase the export of lipids from ABCA1+ cells, causing improved or preventing dysregulated lipid transport. The methods described herein may include replacement of deficient lipid transport function by intravitreal or systemic injection of the therapeutic peptide mimetics of apolipoprotein functions. These therapeutic methods may therefore address LDLR, GAG and/or SR-B1-dependent mechanisms of lipid import into cells. These therapeutic methods may therefore also address ABCA1-dependent or ABCA1-independent mechanisms of lipid export from cells. These methods and compositions (e.g., therapeutic peptides) may modulate the patient's cholesterol homeostasis. The therapeutic peptides may generally be small (e.g., <50 amino acids, <49 amino acids, <48 amino acids, etc.), amphipathic, and may package lipids. The therapeutic peptides are engineered to bind and sequester lipids and may deposit the lipid into cells via uptake receptors or enable the export of lipids from cells via export receptors. For example, these therapeutic peptides may improve lipid clearance from drusen through increased interactions with LDLR and other lipid uptake pathways. As an additional example, these therapeutic peptides may reduce drusen burden through increased interactions with ABCA1 and other lipid export pathways, to restore natural lipid transport homeostasis and clearance mechanisms. Since drusen represent a major risk factor for AMD disease progression, therapeutic peptides having the potential to decrease drusen formation will likely improve patient outcomes. These peptides may be synthesized synthetically. In some examples, these compositions (e.g., therapeutic peptides) may be formulated with one or more pharmaceutically acceptable carriers.

Also described herein are methods of treating a subject having a disorder associated with undesired activity of lipid regulatory pathways, comprising the step of administering to the subject any of the compositions disclosed herein.

In some examples, the disclosure provides for a method of treating a subject having age-related macular degeneration (AMD), comprising the step of administering to the subject any of the compositions disclosed herein. In some examples, the composition is administered intravitreally. In some examples, the subject is a human. In some examples, the human is at least 40 years of age. In some examples, the human is at least 50 years of age. In some examples, the human is at least 65 years of age. In some examples, the composition is administered locally. In some examples the composition is administered systemically. In some examples, the composition has an amino acid sequence of any one of SEQ ID NOs: 35-39, 87, 101 or 114. For example, described herein are polypeptides for use in treating age-related macular degeneration (AMD) having a peptide sequence that is less than 80 amino acids long and has 65% or more (e.g., 70% or more, 80% or more, 85% or more, 90% or more, etc.) homology to one of SEQ ID NO.: 5, 6, 7, 8, 9, 10, 11, 12, 13, 14, 15, 16, 17, 18, 19, 20, 21, 22, 23, 24, 25, 26, 27, 28, 29, 30, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 7, 74, 75, 76, 77, 78, 79, 80, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177 or 178. In particular, described herein are polypeptides for use in treating age-related macular degeneration (AMD) having a peptide sequence that is less than 80 amino acids long and has 65% or more (e.g., 70% or more, 80% or more, 85% or more, 90% or more, etc.) homology to one of SEQ ID NO.: 87, 101 or 114.

In some examples, a polypeptide for use in treating age-related macular degeneration (AMD) may have a peptide sequence that is at least 85% homologous to one of SEQ ID NO.: 84, 86, 87, 101, 112, 114, or 116.

In some examples, the polypeptide is more than 90% homologous to one of SEQ ID NO.: 84, 86, 87, 101, 112, 114, or 116.

A polypeptide for use in treating age-related macular degeneration (AMD) may have a peptide sequence that is at least 85% homologous to SEQ ID NO.: 35, 36, 37, 38 or 39.

Described herein are examples of one or more polypeptides for use in treating age-related macular degeneration (AMD) having a peptide sequence of one of SEQ ID NO.: 84, 86, 87, 101, 112, 114, or 116.

Described herein are examples of one or more polypeptides for use in treating age-related macular degeneration (AMD) having a peptide sequence of one of SEQ ID NO.: 35, 36, 37, 38 or 39.

Described herein are peptide sequences relating to apolipoprotein mimetics for use in treating age-related macular degeneration (AMD) having ATP binding cassette transporter binding activity comprising a sequence that is at least 65% homologous to one or more of SEQ ID No.: 84, 86, 87, 101, 112, 114, or 116.

Described herein are one or more polypeptides for use in treating age-related macular degeneration (AMD) having cholesterol efflux regulatory protein (CERP) binding activity comprising a sequence that is at least 65% homologous to one or more of SEQ ID NO.: 84, 86, 87, 101, 112, 114, or 116.

Described herein are one or more polypeptides for use in treating age-related macular degeneration (AMD) having transporter protein binding activity comprising a sequence that is at least 65% homologous to one or more of SEQ ID NO.: 84, 86, 87, 101, 112, 114, or 116.

Described herein are one or more polypeptides for use in treating age-related macular degeneration (AMD) having ATP binding cassette transporter binding activity comprising a sequence that is at least 65% homologous to one or more of SEQ ID NO.: 84, 86, 87, 101, 112, 114, or 116.

A polypeptide for use in treating age-related macular degeneration (AMD) may have a peptide sequence that is at least 85% homologous to one of SEQ ID NO.: 25, 27 or 29. In some examples the polypeptide is more than 90% homologous to one of SEQ ID NO.: 25, 27 or 29.

A polypeptide for use in treating age-related macular degeneration (AMD) may have a peptide sequence of one of SEQ ID NO. 25, 27 or 29. For example, a polypeptide for use in treating age-related macular degeneration (AMD) may have 80 or fewer amino acids, wherein an N-terminal end of the polypeptide has 65% or more (e.g., 70% or more, 80% or more, 85% or more, 90% or more, etc.) homology to SEQ ID NO. 8.

In some examples, a polypeptide for use in treating age-related macular degeneration (AMD) has an N-terminal end of the polypeptide with 65% or more (e.g., 70% or more, 80% or more, 85% or more, 90% or more, etc.) homology to SEQ ID NO. 29, wherein the first eleven amino acids of the polypeptide have four or fewer (e.g., three or fewer, two or fewer, or one) substitutions as compared to SEQ ID NO. 29.

For example, a polypeptide for use in treating age-related macular degeneration (AMD) may have a lipid-dependent Low Density Lipoprotein Receptor (LDLR) binding activity comprising a sequence that is at least 65% homologous to one or more of SEQ ID NO.: 25, 27 or 29. In some examples the polypeptide for use in treating age-related macular degeneration (AMD) has a lipid-dependent Low Density Lipoprotein Receptor (LDLR) binding activity comprising a sequence that is at least 65% homologous to one or more of SEQ ID NO.: 5, 7, 9. 13, 18 and 29.

A polypeptide for use in treating age-related macular degeneration (AMD) having 80 or fewer amino acids, wherein an N-terminal end of the polypeptide has 65% or more homology to an L-confirmation shown in SEQ ID NO. 8 or wherein a C-terminal end of the polypeptide has 65% or more homology to a D-confirmation of SEQ ID NO. 8.

A polypeptide for use in treating age-related macular degeneration (AMD) may have 80 or fewer amino acids, wherein an N-terminal end of the polypeptide is homologous with fewer than 2 amino acid substitutions or deletions to an L-confirmation shown in SEQ ID NO. 8 or wherein a C-terminal end of the polypeptide is homologous with fewer than 2 amino acid substitutions or deletions to a D-confirmation of SEQ ID NO. 8.

A polypeptide for use in treating age-related macular degeneration (AMD) may have 80 or fewer amino acids, whereas an N-terminal end of the polypeptide has 65% or more homology to SEQ ID NO. 8.

A polypeptide for use in treating age-related macular degeneration (AMD) may have an N-terminal end of the polypeptide with 65% or more homology to SEQ ID NO. 29, wherein the first eleven amino acids of the polypeptide have two or fewer substitutions as compared to SEQ ID NO. 29.

A polypeptide for use in treating age-related macular degeneration (AMD) may have 80 or fewer amino acids, wherein an N-terminal end of the polypeptide has is 65% or more (e.g., 70% or more, 80% or more, 85% or more, 90% or more, etc.) homologous to SEQ ID NO. 27.

Any of the polypeptide described herein may have a lipid-dependent Low Density Lipoprotein Receptor (LDLR) binding activity such that the polypeptide binds to LDLR in the presence of lipid at least two-fold (e.g., at least 2.5-fold, at least 3-fold, at least 5-fold, at least 10-fold, etc.) greater than the polypeptide binds to LDLR in the absence of lipid.

Any of the therapeutic peptides described herein may increase the efflux of lipid from cells (e.g. microglial cells or retinal pigment epithelial cells) via ATP-binding cassette transporter ABCA1 and increase the presence of ABCA1 on the membrane of cells following pro-inflammatory stimulation.

Any of the therapeutic peptides described herein may include N-term acetylation and C-term Amidation. The therapeutic peptides described herein are also intended to include both L and D forms of the peptides described herein.

Also described herein are pharmaceutical compositions for use in prevention or treatment of age-related macular degeneration (AMD) in a patient, wherein the composition comprises any of the polypeptides described herein and a pharmaceutically acceptable excipient. The composition may be for administration by intraocular injection and/or intravascular (IV) injection, and/or subcutaneous (SC) injection. The pharmaceutical composition may include two or more of the polypeptides described above.

For example, a method of treating or preventing age-related macular degeneration (AMD) in a patient using the polypeptides or pharmaceutical compositions described may be used when the patient shows signs or symptoms of AMD. These methods of treating or preventing age-related macular degeneration (AMD) in a patient using the polypeptides or pharmaceutical compositions described herein may be for treatment of early-stage AMD. Any of these methods of treating a patient for age-related macular degeneration (AMD) may include delivering the polypeptide or composition into the patient's eye, e.g., by intraocular injection (and/or by intravascular injection and/or by subcutaneous injection, etc.). As mentioned, the method may include delivering more than one of the polypeptides or compositions described herein.

In particular, described herein are peptides for treating or preventing age-related macular degeneration (AMD) in a patient include or are derived from the peptide having the sequence shown below:

(SEQ ID. NO.: 87)
DAWERFRALFKELADYFR

These polypeptides are shown herein to have surprisingly beneficial properties for treating AMD. As illustrated herein, polypeptides having a sequence that is at least 65% homologous (e.g., including six or fewer conservative and/or conservative hydrophobicity substitutions) and that display one or more demonstrable properties such as: a hydrophobic moment (μH) of 0.65 or greater, ATP binding cassette transporter membrane stabilization and agonist activity, transporter protein binding activity and/or a cholesterol efflux of 17.5% or greater, may be therapeutically effective to treat AMD. Non-limiting examples of polypeptides that are homologous to T-087 and demonstrate either sustained or improved activity in key assays include: T-152, T-160, T-161, T-163, and T-172.

Thus, described herein are polypeptides for use in treating age-related macular degeneration (AMD) having a peptide sequence that is 65% or more homologous to SEQ ID NO.: 87, wherein the polypeptide comprises a helical coil having a hydrophobic moment, μH, of 0.65 or greater. As mentioned, in the framework of the T-087 polypeptide, the hydrophobic moment correlates with the percent of cholesterol efflux, such that a hydrophobic moment, μH, of 0.65 or greater typically results in an increase in cholesterol efflux of greater than 15% (e.g., 16% or greater, 17% or greater, 17.5% % or greater, 18% or greater, etc.).

For example, also described herein are polypeptides for use in treating age-related macular degeneration (AMD) having a peptide sequence that has 65% or more homology to SEQ ID NO.: 87, wherein the polypeptide has ATP binding cassette transporter membrane stabilization and agonist activity.

Also described herein are polypeptides for use in treating age-related macular degeneration (AMD) having a sequence that is at least 65% homologous to SEQ ID NO.: 87, wherein the polypeptide has transporter protein binding activity.

Any of these polypeptides may have a hydrophobic moment, μH, of 0.65 or greater, resulting in a cholesterol efflux of 17.5% or greater. Any of these polypeptides may have ATP binding cassette transporter binding activity.

In general, for any of these polypeptides based on T-087, any peptides residues that differ from the sequence of SEQ ID NO.: 87 in positions 2-7, 9 and 10-17 are conservative substitutions or conservative hydrophobicity substitutions having a hydrophobicity value that is within 0.25 of the hydrophobicity value of a peptide residue in a corresponding position of SEQ ID NO.: 87, as calculated using the method of Fauchere and Pliska, and wherein any differing peptide residues in positions 1, 8, 10 and 18 are any amino acid. For example, the polypeptide sequence may be one of SEQ. ID. NO.: 87, 152, 153, 154, 155, 156, 157, 158, 159, 160, 161, 162, 163, 164, 165, 166, 167, 168, 169, 170, 171, 172, 173, 174, 175, 176, 177 or 178. In particular, the polypeptide sequence may be one of SEQ. ID. NO.: 87, 152, 160, 161, 163 or 172.

In any of these examples, the polypeptide sequence may be 70% or more (e.g., 80% or more, 85% or more, 90% or more, etc.) homologous to SEQ ID NO: 87. As mentioned, in any of these polypeptide sequences based on T-087, the polypeptide sequence may be as shown is SEQ ID NO.: 35 or 36, wherein X may be any amino acid.

Also described herein are peptides for treating or preventing age-related macular degeneration (AMD) in a patient include or are derived from the peptide having the sequence shown below:

(SEQ ID. NO.: 101)
RSGADALESALKELKRFIREWT

These polypeptides are also shown herein to have surprisingly beneficial properties for treating AMD. Polypeptides having a sequence that is at least 65% homologous (e.g., including eight or fewer conservative and/or conservative hydrophobicity substitutions) and that display one or more demonstrable properties such as: a hydrophobic moment (μH) of 0.6 or greater (which may confer greater ABCA1 stability), ATP binding cassette transporter membrane stabilization and agonist activity, transporter protein binding activity and/or an overall hydrophobicity of 0.198 or greater (which may confers greater cholesterol efflux). Non-limiting examples of polypeptides that are homologous to T-087 and demonstrate either sustained or improved activity in key assays include: T-122, T-123, T-129, T-136 and T-139.

For example, described herein are polypeptides for use in treating age-related macular degeneration (AMD) having a peptide sequence that is 65% or more homologous to SEQ ID NO.: 101, wherein the polypeptide comprises a helical coil having a hydrophobic moment, pH, of 0.6 or greater. For example described herein are polypeptides for use in treating age-related macular degeneration (AMD) having a peptide sequence that has 65% or more homology to SEQ ID NO.: 101, wherein the polypeptide has ATP binding cassette transporter membrane stabilization and agonist activity. Also described herein are polypeptides for use in treating age-related macular degeneration (AMD) having a sequence that is at least 65% homologous to SEQ ID NO.: 101, wherein the polypeptide has transporter protein binding activity. In some examples a polypeptide for use in treating age-related macular degeneration (AMD) may have an overall hydrophobicity of 0.198 or greater, resulting in an enhanced cholesterol efflux. In any of these examples, the polypeptide may have a hydrophobic moment, μH, of 0.6 or greater, resulting in an ABCA1 stability that is greater than forty percent of the ABCA1 stability of the polypeptide of SEQ ID NO.: 101. The polypeptide may have ATP binding cassette transporter binding activity.

In any of the polypeptides that are 65% or more homologous that substitute any peptides residues that differ from the primary sequence (e.g., the sequence of SEQ ID NO.: 101), e.g., in positions 1, 4-8, 10-15, 17-18, or 20-22 as a conservative substitution or a conservative hydrophobicity substitution having a hydrophobicity value that is within 0.25 of the hydrophobicity value of a peptide residue in a corresponding position of SEQ ID NO.: 101, as calculated using the method of Fauchere and Pliska, and wherein any differing peptide residues in positions 2, 3, 9, 16 and 19 are any amino acid. For example, the polypeptide sequence may be one of SEQ. ID. NO.: 101, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150 or 151. Specifically, the polypeptide sequence may be one of SEQ. ID. NO.: 101, 122, 123, 129, 136 or 139. The polypeptide sequence may be 70% or more homologous to SEQ ID NO: 101 (e.g., 75% or more homologous, 80% or more, 85% or more, 90% or more, etc.).

Also described herein are polypeptides based on the framework of the T-114 polypeptide. For example, described herein are polypeptides for use in treating age-related macular degeneration (AMD) having a peptide sequence that is 65% or more homologous to SEQ ID NO.: 114, wherein the polypeptide has potent lipid-dependent binding to the Low-Density Lipoprotein Receptor. The polypeptide for use in treating age-related macular degeneration (AMD) may have a sequence that is at least 65% homologous to SEQ ID NO.: 114, wherein the polypeptide has transporter protein binding activity. In any of these polypeptides, the polypeptide may also include ATP binding cassette transporter binding activity.

As mentioned above, also described herein generally are pharmaceutical compositions for use in prevention or treatment of age-related macular degeneration (AMD) in a patient, wherein the composition comprises the polypeptide described in any of the examples above, and a pharmaceutically acceptable excipient. The composition may be for administration by intraocular injection, intravascular (IV) injection, and/or subcutaneous (SC) injection. Any of these pharmaceutical compositions may include two or more of the different polypeptides described herein.

Also described herein are methods of treating or preventing age-related macular degeneration (AMD) in a patient using any of these polypeptide or pharmaceutical composition wherein the prevention or treatment is prevention of said AMD, and wherein the patient is diagnosed as having a propensity to develop AMD. For example, a method of treating or preventing age-related macular degeneration (AMD) in a patient may include using any of the polypeptides described herein, wherein the prevention or treatment is treatment and the patient shows signs or symptoms of AMD. A method of treating or preventing age-related macular degeneration (AMD) in a patient using one or more of the engineered polypeptide or pharmaceutical composition described herein may include treatment of early-stage AMD. A method of treating a patient for age-related macular degeneration (AMD) may include delivering the polypeptide or composition into the patient's eye, e.g., by intraocular injection, intravascular (IV) injection, and/or subcutaneous (SC) injection. Delivering may comprise delivering more than one of the polypeptides or compositions described herein. As mentioned, in some examples, the patient may be, e.g., 40 years old or older.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative examples, and the accompanying drawings of which:

FIGS. 1A-1I are a listing of the peptides, including control peptides (T-001-T-004, T-031-T-034 and T-081-T-083) and therapeutic peptides (T-005-T-030; and T-040-T-178) described herein, also showing hydrophobic moment. Hydrophobic moment is defined in Eisenberg (Eisenberg et al. 1982. The helical hydrophobic moment: a measure of the amphiphilicity of a helix. Nature).

FIGS. 2A-2D show results from DMPC solubility assays measuring solubilization of 0.5 mM 1,2-dimyristoyl-sn-glycero-3-phosphocholine (DMPC) for selected polypeptides shown in FIG. 1A-1I with a peptide concentration of 100 μM.

FIGS. 3A-3C are graphs showing results of solubilization of 0.5 mM DMPC for control (T-001, T-002, T-004, and T-031) as well as therapeutic polypeptides (T-025, T-027, FIG. 3A; T-051, T-052, T-054, T-059, FIG. 3B; T-062, T-063, T-065, T-069, T-080, FIG. 3C) as described herein across a range of peptide concentrations.

FIGS. 4A-4B graphically illustrate solubilization of 1 mM DMPC for select peptides shown in FIGS. 1A-1I as described herein over a range of peptide concentrations.

FIGS. 5A-5D are graphs showing results of ARPE-19 cell lysis assays for select control (T-031, T-032, T-033 and T-034) and therapeutic polypeptides (T-006, T-007, T-009, T-010, T-011, T-012, T-013, T-014, T-018, T-019, T-021, T-025, T-027, T-028, T-029, T-024, T-026, and T-030) at a single concentration (10 μM) as described herein.

FIG. 6 is a graph showing ARPE-19 cell lysis titration curves for control (T-001, T-002, T-004 and T-034) as well as select therapeutic polypeptides (T-025 and T-027) as described herein across a range of peptide concentrations.

FIGS. 7A-7D illustrate the results of an ARPE-19 cell viability assay for select peptides shown in FIG. 1A-1I at a single concentration (100 μM).

FIGS. 8A-8D show graphs summarizing the results of human red blood cell (hRBC) lysis assays using control peptides (T-001, T-002, T-004, T-031, T-032, T-033 and T-034) as well as therapeutic polypeptides (T-006, T-007, T-009, T-010, T-011, T-012, T-013, T-014, T-024, T-026, T-030, T-018, T-019, T-021, T-025, T-027, T-028, T-029) as described herein at a single concentration (10 μM).

FIGS. 9A-9B illustrate the results of hRBC lysis assays for select peptides shown in FIGS. 1A-1I at a single concentration (10 μM).

FIG. 10 is a graph illustrating hRBC lysis titration curves for select peptides shown (control: T-001, T-002, T-004 and T-034) and therapeutic polypeptides: T-025 and T-027) across a range of concentrations.

FIG. 11 is a graph summarizing the results of hRBC cell lysis titration curves using control peptides (T-001 and T-034) as well as therapeutic polypeptides (T-025, T-086, T-087, T-101, T-112, T-114, and T-116) as described herein across a range of peptide concentrations.

FIG. 12 is a graph of ARPE-19 cholesterol import for select peptides shown in FIGS. 1A-1I at a single concentration (10 μM).

FIG. 13 is a graph summarizing the results of ARPE-19 GAG-dependent cholesterol import assays using control peptide (T-002) as well as therapeutic polypeptides (T-013, T-021, T-025, T-027, T-028, T-029, and T-030) at a single concentration (10 μM) as described herein.

FIGS. 14A-14B are graphs summarizing ARPE-19 cholesterol import assays for select peptides shown in FIGS. 1A-1I at a single concentration (10 μM).

FIG. 15 is a graph showing ARPE-19 cholesterol import titration curves for control peptides (T-001, T-002, T-004 and T-032) and selected therapeutic polypeptides (T-025 and T-027) over a range of peptide concentrations.

FIG. 16 is a graph showing ARPE-19 cholesterol import titration curves for control peptides (T-001, T-002) and selected therapeutic polypeptides (T-086, T-087, T-101, T-112, T-114 and T-116) as described herein across a range of peptide concentrations.

FIG. 17 is a graph showing HepG2 cholesterol import screening data for select peptides shown in FIGS. 1A-1I at a single concentration (10 μM).

FIG. 18 is a graph showing HepG2 cholesterol import screening data for select peptides shown in FIGS. 1A-1I at a single concentration (10 μM).

FIG. 19 is a graph showing screening results for select peptides shown in FIG. 1A-1I in a HepG2 GAG dependent cholesterol import assay at a single concentration (10 μM).

FIG. 20 shows examples of HepG2 cholesterol import titration curves for control (T-001, T-002, T-004 and T-032) and therapeutic peptides (T-025 and T-027).

FIG. 21 shows examples of HepG2 cholesterol import titration curves for control (T-001, T-002) and therapeutic peptides (T-086, T-087, T-101, T-112, T-114 and T-116).

FIG. 22 graphically summarizes the results of lipid-dependent LDLR binding (SPR) assays for recombinant ApoE2 and ApoE4, as well as control peptides (T-001, T-002, T-031, T-032, T-033 and T-034) and select test peptides shown in FIG. 1A-1I at a single concentration (1 UM).

FIG. 23 is a table showing the binding kinetics results of LDLR binding assays (SPR), high surfactant preparation as described herein for select peptides shown in FIG. 1A-1I.

FIGS. 24A-24B are tables showing the binding kinetics results of LDLR binding assays (SPR), lipid-dependent binding with a low-surfactant preparation as described herein for select peptides shown in FIG. 1A-1I.

FIGS. 25A-25O are graphs showing activity of non-oxidized and oxidized lipids binding to various immobilized peptides from the list of FIGS. 1A-1I (SPR).

FIGS. 26A-26B summarize results from LDL oxidation assays. FIG. 26A is a graph showing the change in LDL oxidation over time as measured by UV-spectrophotometry for select peptides shown in FIG. 1A-1I at a single concentration (25 μM). FIG. 26B is a bar graph showing LDL oxidation rate calculated as maximum absorbance divided by the length of the lag period for select peptides shown in FIG. 1A-1I at a single concentration (25 μM).

FIGS. 27A-27B are graphs showing results of HMC3 cholesterol efflux assays for select peptides shown in FIG. 1A-1I at a single concentration (20 μM).

FIG. 28 summarizes results of HMC3 cholesterol efflux assays (as compared with control) for variants of peptide T-101 (peptides T-121 through T-151) as described herein at a single concentration (20 μM).

FIG. 29 summarizes results of ARPE-19 cholesterol efflux assays (as compared with control) for variants of peptide T-087 (peptides T-152 through T-178) as described herein at a single concentration (20 μM).

FIGS. 30A-30J are graphs of HMC3 cholesterol efflux titration curves for select peptides shown in FIG. 1A-1I at a range of peptide concentrations.

FIGS. 31A-31B illustrate the results of cholesterol efflux assays in ARPE-19 cells following siRNA knockdown of various ATP-binding cassette family members as well as SR-B1 for peptides T-087 (FIG. 31A) and T-101 (FIG. 31B) at a single concentration (20 μM).

FIG. 32 is a graph of the cholesterol efflux in iPS-RPE cells for peptides T-087 and T-101 as described herein at a single concentration (10 μM).

FIGS. 33A-33C illustrate the results of ABCA1 stability testing assays using J774 cells. FIG. 33A shows a representative western blot of ABCA1 membrane levels following treatment with buffer control or with peptides T-101 or T-087 (at 10 μM), or with positive control protein ApoA1 (at 350 nM) after washing out 8-Br-cAMP. FIG. 33B is a graph showing quantified band densitometry for membrane ABCA1 following treatment with either buffer control or with peptides T-101 or T-087 (at 10 μM). FIG. 33C graphically shows the change in the percentage of membrane ABCA1 at different concentrations of peptides T-101 or T-087 as described herein.

FIGS. 34A-34B illustrate results from ABCA1 stability assays using ARPE-19 cells treated with mCRP and peptides T-101 and T-087. FIG. 34A shows western blot data from cells treated with 10 μg/mL mCRP and peptide T-101 and T-087 at 20 μM. FIG. 34B is a graph showing the concentration-response of peptides T-087 and T-101 in this assay.

FIG. 35 is a graph showing results of ABCA1 stability assays using ARPE-19 cells for peptide T-087 and variations of peptide T-087 as described herein at a single concentration (20 UM).

FIG. 36 is a graph showing results of ABCA1 stability assays using ARPE-19 cells for peptide T-101 and variations of peptide T-101 as described herein at a single concentration (20 μM).

FIG. 37 is a graph illustrating the positive correlation of cholesterol efflux from ARPE-19 cells (shown in FIG. 29) and hydrophobic moment for variants of peptide T-087 (T-152-T178). Linear regression was used to determine slope deviation from zero.

FIG. 38A graphically shows the positive correlation of cholesterol efflux from HMC3 cells (shown in FIG. 28) and hydrophobicity for peptides T-121 to T-151 (variants of peptide T-101). Hydrophobicity is defined in Fauchere and Pliska (Fauchere and Pliska, 1983. Hydrophobic Parameters II of Amino-Acid Side Chains from the Partitioning of N-Acetyl-Amino-Acid Amides. Eur J Med Chem). Linear regression was used to determine slope deviation from zero.

FIG. 38B illustrates the positive correlation of ABCA1 stability in ARPE-19 cells (shown in FIG. 36) and hydrophobic moment for peptides T-121 to T-151. Linear regression was used to determine slope deviation from zero.

FIGS. 39A-39E show representative H&E stained sections through adult C57BL/6 mouse eyes treated with a subset of the peptides described herein (T-087, T-101, T-112, and T-114) showing in vivo tolerability seven days following intravitreal injection of 1 μL peptide at 520 μM.

FIGS. 40A-40B illustrate the evaluation of peptide-mediated reduction in sub-RPE BODIPY+ lipid deposition in ApoE^{-/-} Mice for peptides T-087 and T-101. Peptides (1 μL at 520 μM or 260 μM) were delivered via intravitreal injection and sub-RPE lipid deposition was measured at 14 days post-injection by BODIPY staining of retinal sections.

FIGS. 41A-41B indicates residues of peptides T-087 (FIG. 41A) and T-101 (FIG. 41B) (shown in the boxes) that may be the same or substituted for conservative amino acid substitutions to maintain the desired therapeutic activities of the peptide, including for safety, cholesterol efflux capacity, and ABCA1 stabilization capacity. Residues shown without boxes indicate positions where any amino acid may be substituted to maintain the desired therapeutic activities of the peptide, including for safety, cholesterol efflux capacity, and ABCA1 stabilization capacity.

### DETAILED DESCRIPTION

The compositions and methods described herein may be used to treat AMD, and in particular, AMD patients whose disease is primarily driven by dysfunction of lipid transport and processing. For example, synthetically engineered mimetic peptides described herein provide therapeutic opportunity to safely and effectively address pathophysiology, such as dysregulated lipid homeostasis.

In particular, apolipoproteins are endogenously synthesized in response to intracellular and extracellular lipid transport and processing needs. Generally, apolipoproteins are a family of amphipathic molecules capable of binding and trafficking lipids into and out of cells and peripheral tissue as part of lipid homeostatic mechanisms. For example, apolipoproteins are a component of HDL/LDL molecules. Apolipoprotein E (ApoE) is generally associated with LDL, while apolipoprotein A1 (ApoA1) is generally associated with HDL.

Cellular uptake of lipids, such as cholesterol, may be due to metabolic requirements of the cell or in response to elevated extracellular cholesterol concentration. Apolipoprotein E (e.g. ApoE) is generally associated with mechanisms relating to cholesterol uptake. Low-density lipoprotein receptor (LDLR) is a surface receptor with a high affinity to ApoE. Retinal pigmented epithelium (RPE) cells have been shown to express LDLR, which enables a pathway for the uptake of cholesterol from cholesterol-rich LDL/ApoE complexes.

Alternatively, cholesterol efflux is the mechanism by which cholesterol is excreted from within a cell to an extracellular environment through one or more transporter proteins (e.g., ATP binding cassette transporters or SR-B1). The transporters efflux cholesterol that is then bound to lipid poor ApoAI as a component in the formation of HDL. As ApoA1 accumulates cholesterol from the transporter proteins, HDL becomes saturated and continues with RCT as part of the lipid transport and processing mechanism for cholesterol homeostasis and lipid regulation.

Genotypic variations, environmental factors, and senescence may contribute to lipid homeostatic dysregulation associated with impaired apolipoprotein activity. Described herein are synthetically engineered mimetic peptides with lipid transport and processing activity as shown in detail below. These synthetic peptides may describe families of peptides (including conservative substations of peptides as described herein) that display remarkable therapeutic efficacy.

In some examples, the dysfunction of lipid transport and processing may relate to impaired lipid efflux from one or more cells or tissues related to the pathogenesis of AMD. For example, RPE cell-specific deletion of the ABCA1 gene in mouse models leads to aberrant lipid accumulation, retinal inflammation and RPE/photoreceptor degeneration (PMID 30864945). Apolipoproteins, and in particular Apolipoprotein A1 (ApoA1), act as regulators of lipid processing and trafficking of lipid molecules to maintain cholesterol homeostasis. ApoA1 may interact with one or more one or more cholesterol efflux regulatory proteins, such as ABCA1, ABCG1 or SR-B1. The cholesterol efflux regulator proteins transport cholesterol to ApoA1 that binds the cholesterol in formation of nascent HDL. As cholesterol is accumulated, the lipid-rich HDL is circulated to the liver for lipid deposition and metabolism.

In some examples, wherein the primary dysfunction in lipid processing relates to the accumulation of lipids via improper exocytosis of lipids by one or more cholesterol regulatory proteins, a patient with AMD may benefit from administration of one or more of the therapeutic peptides described herein (e.g., included in SEQ ID NOS. 5-30; or 35-178). For example, where there is an excessive rate of cholesterol import and corresponding intracellular lipid concentrations, administration of one or more of the therapeutic peptides described herein (e.g., included in SEQ ID NOS. 5-30; or 35-178) establish or restore effective lipid transport and processing mechanisms mediated by one or more apolipoproteins. As will be described in greater detail herein, some of these peptides may be more efficacious than others, however, in general they may provide therapeutic use.

In some examples, including (but not limited to) examples where the primary contributing factor is due to a one or more dysfunctions relating to a lipid transport and processing pathway, the patient may be treated with one or more therapeutic peptides as described herein, or modified versions of these peptides (e.g., included in SEQ ID NOS. 5-30; or 35-178). In particular, these one or more therapeutic peptides are those that decrease lipid accumulation in Bruch's membrane, in subretinal spaces, or in reticular pseudodrusen, to prevent or reduce AMD-related effects. For example, the methods described herein may replace deficient lipid regulation in Bruch's membrane by intravitreal injection of one or more of the therapeutic peptides described herein.

The disclosure herein provides compositions and methods for treating, preventing, or inhibiting diseases of the eye. For example, the disclosure herein provides engineered therapeutic peptides (therapeutic polypeptides) that selectively bind lipid, solubilize lipid, activate lipid efflux mechanisms via ABCA1 and related transporters and improve cholesterol homeostasis to decrease drusen formation. The disclosure provides methods of treating, preventing, or inhibiting diseases of the eye by intraocularly (e.g., intravitreally) administering an effective amount of these compositions of the disclosure to treat or prevent diseases of the eye using the methods provided herein. Diseases of the eye that may be treated or prevented using these methods include but are not limited to glaucoma, macular degeneration (e.g., age-related macular degeneration, AMD), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, diabetes, systemic infections, or underlying carotid disease, a toxic compound or agent, or photically).

The disclosure herein provides compositions and methods for treating, preventing, or inhibiting diseases of the eye. For example, the disclosure herein provides engineered therapeutic peptides (therapeutic polypeptides) that selectively bind lipid, solubilize lipid, increase LDLR binding activity, increase lipid import activity via LDLR and related receptors and improve cholesterol homeostasis to decrease drusen formation. The disclosure herein also provides engineered therapeutic peptides that selectively activate ABCA1 and promote the efflux of cholesterol from cells through an ABCA1 specific mechanism, to improve cholesterol homeostasis and decrease drusen formation and progression. The disclosure provides methods of treating, preventing, or inhibiting diseases of the eye by intraocularly (e.g., intravitreally) administering an effective amount of these compositions of the disclosure to treat or prevent diseases of the eye using the methods provided herein. Diseases of the eye that may be treated or prevented using these methods include but are not limited to, glaucoma, macular degeneration (e.g., age-related macular degeneration, AMD), diabetic retinopathies, inherited retinal degeneration such as retinitis pigmentosa, retinal detachment or injury and retinopathies (such as retinopathies that are inherited, induced by surgery, trauma, an underlying aetiology such as severe anemia, SLE, hypertension, blood dyscrasias, diabetes, systemic infections, or underlying carotid disease, a toxic compound or agent, or photically).

Unless otherwise defined herein, scientific and technical terms used in this application shall have the meanings that are commonly understood by those of ordinary skill in the art.

Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, NY (2001); Ausubel et al., Current Protocols in Molecular Biology, John Wiley & Sons, NY (2002); Harlow and Lane Using Antibodies: A Laboratory Manual, Cold Spring Harbor Laboratory Press, Cold Spring Harbor, NY (1998); Coligan et al., Short Protocols in Protein Science, John Wiley & Sons, NY (2003); Short Protocols in Molecular Biology (Wiley and Sons, 1999).

Enzymatic reactions and purification techniques are performed according to manufacturer's specifications, as commonly accomplished in the art or as described herein. The nomenclatures used in connection with, and the laboratory procedures and techniques of, analytical chemistry, biochemistry, immunology, molecular biology, synthetic organic chemistry, and medicinal and pharmaceutical chemistry described herein are those well-known and commonly used in the art. Standard techniques are used for chemical syntheses, and chemical analyses.

Where aspects or examples of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the example disclosed. Exemplary methods and materials are described herein, although methods and materials similar or equivalent to those described herein can also be used in the practice or testing of the present disclosure. The materials, methods, and examples are illustrative only and not intended to be limiting.

### Definitions

The following terms, unless otherwise indicated, shall be understood to have the following meanings:

As used herein, “residue” refers to a position in a protein and its associated amino acid identity. As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, examples wherein phosphate is replaced by P(0)S (“thioate”), P(S)S (“dithioate”), (O)NRi (“amidate”), P(0)R, P(0)OR′, CO or CH2 (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

“Homologous,” in all its grammatical forms and spelling variations, refers to the relationship between two proteins that possess a common sequence, including protein sequence from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” particularly (but not exclusively) when modified with a percentage may refer to sequence similarity and may or may not relate to a common evolutionary origin.

The term “sequence similarity,” in all its grammatical forms, refers to the degree of identity or correspondence between nucleic acid or amino acid sequences that may or may not share a common evolutionary origin. “Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

The term “conservative substitution” refers to substitution of an amino acid in a polypeptide with a functionally, structurally or chemically similar natural or unnatural amino acid. In certain embodiments, the following groups each contain natural amino acids that are conservative substitutions for one another: 1) Glycine (G), Alanine (A); 2) Aspartic acid (D), Glutamic acid (E); 3) Asparagine (N), Glutamine (Q); 4) Arginine (R), Lysine (K), Histidine (H); 5) Isoleucine (I), Leucine (L), Methionine (M), Valine (V), Alanine (A); 6) Phenylalanine (F), Tyrosine (Y), Tryptophan (W); and 7) Serine(S), Threonine (T), Cysteine (C).

The peptides described herein may include substitutions that do not disrupt the helical structure of the peptides in a manner that disrupts the function as described below. In particular, the peptides (e.g., T-087, T-101, etc.) may include one or more substitutions such that the hydrophobicity of the substituted residue is within 0.25 of the hydrophobicity value of the original amino acid (referred to herein as conservative hydrophobicity substitutions). For example: (1) Alanine (A) can be substituted with Threonine (T) or Histidine (H); (2) Glutamic acid (E) can be substituted with Asparagine (N) or Aspartic acid (D); (3) Arginine (R) can be substituted with Aspartic acid (D) or Lysine (K); (4) Phenylalanine (F) can be substituted with Cysteine (C), Leucine (L) or Isoleucine (I); (5) Leucine (L) can be substituted with Isoleucine (I), Phenylalanine (F) or Cysteine (C); (6) Lysine (K) can be substituted with Aspartic acid (D) or Arginine (R); (7) Aspartic acid (D) can be substituted with Asparagine (N), Glutamic acid (E), Lysine (K), or Arginine (R); (8) Tyrosine (Y) can be substituted with Proline (P); (9) Serine(S) can be substituted with Glutamine (Q), Glycine (G) or Histidine (H); (10) Isoleucine (I) can be substituted with Leucine (L) or Phenylalanine (F); (11) Threonine (T) can be substituted with Alanine (A) or Histidine (H); and (12) Serine(S) can be substituted with Histidine (H), Serine(S) or Glutamine (Q). Hydrophobicity is determined as using the techniques of Fauchere and Pliska (e.g., Fauchere and Pliska, Eur J Med Chem, 1983).

Alternatively or additionally, the peptides described herein may include substitutions such that the size of the substituted residue is approximately the same and/or is the next closest in size to the original amino acid (referred to herein as conservative size substitutions). For example: Aspartic acid (D) may be substituted with Asparagine (N) or Lysine (K); Alanine (A) may be substituted with Glycine (G) or Serine(S); Tryptophan (W) may be substituted with Tyrosine (Y); Glutamic acid (E) may be substituted with Glutamine (Q) or Methionine (M); Arginine (R) may be substituted with Phenylalanine (F) or Tyrosine (Y); Phenylalanine (F) may be substituted with Histidine (H) or Arginine (R); Leucine (L) may be substituted with Cysteine (C) or Isoleucine (I); Lysine (K) may be substituted with Aspartic acid (D) or Glutamine (Q); Tyrosine (Y) may be substituted with Arginine (R) or Tryptophan (W); Serine(S) may be substituted with Alanine (A) or Proline (P); Glycine (G) may be substituted with Alanine (A); Isoleucine (I) may be substituted with Leucine (L) or Asparagine (N); Threonine (T) may be substituted with Valine (V) or Cysteine (C).

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature. The therapeutic polypeptides described herein may be isolated.

As used herein, “purify,” and grammatical variations thereof, refers to the removal, whether completely or partially, of at least one impurity from a mixture containing the polypeptide and one or more impurities, which thereby improves the level of purity of the polypeptide in the composition (i.e., by decreasing the amount (ppm) of impurity(ies) in the composition). The therapeutic polypeptides described herein may be referred to as purified.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure. The therapeutic polypeptides described herein may be substantially pure.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In some examples, the subject is a human that is at least 40, 45, 50, 55, 60, 65, 70, 75, 80, 85, 90 or 95 years of age.

In one example, the subject has, or is at risk of developing a disease of the eye. A disease of the eye, includes, without limitation, retinitis pigmentosa, rod-cone dystrophy, Leber's congenital amaurosis, Usher's syndrome, Bardet-Biedl Syndrome, Best disease, retinoschisis, Stargardt disease (autosomal dominant or autosomal recessive), untreated retinal detachment, pattern dystrophy, cone-rod dystrophy, achromatopsia, ocular albinism, enhanced S cone syndrome, diabetic retinopathy, age-related macular degeneration, retinopathy of prematurity, sickle cell retinopathy, Congenital Stationary Night Blindness, glaucoma, or retinal vein occlusion. In another example, the subject has, or is at risk of developing glaucoma, Leber's hereditary optic neuropathy, lysosomal storage disorder, or peroxisomal disorder. In some examples, the subject has shown clinical signs of a disease of the eye.

In some examples, the subject has, or is at risk of developing a renal disease or complication. In some examples, the renal disease or complication is associated with AMD or atypical hemolytic uremic syndrome (aHUS). In some examples, the subject has, or is at risk of developing AMD or aHUS.

Clinical signs of a disease of the eye include, but are not limited to, decreased peripheral vision, decreased central (reading) vision, decreased night vision, loss of color perception, reduction in visual acuity, decreased photoreceptor function, and pigmentary changes. In one example, the subject shows degeneration of the outer nuclear layer (ONL). In another example, the subject has been diagnosed with a disease of the eye. In yet another example, the subject has not yet shown clinical signs of a disease of the eye.

As used herein, the terms “prevent”, “preventing” and “prevention” refer to the prevention of the recurrence or onset of, or a reduction in one or more symptoms of a disease or condition (e.g., a disease of the eye) in a subject as result of the administration of a therapy (e.g., a prophylactic or therapeutic agent). For example, in the context of the administration of a therapy to a subject, “prevent”, “preventing” and “prevention” refer to the inhibition or a reduction in the development or the onset or the progression of a disease or condition (e.g., a disease of the eye), or the prevention of the recurrence, onset, or development of one or more symptoms of a disease or condition (e.g., a disease of the eye), in a subject resulting from the administration of a therapy (e.g., a prophylactic or therapeutic agent), or the administration of a combination of therapies (e.g., a combination of prophylactic or therapeutic agents).

“Treating” a condition or patient refers to taking steps to obtain beneficial or desired results, including clinical results. With respect to a disease or condition (e.g., a disease of the eye), treatment refers to the reduction or amelioration of the progression, severity, and/or duration of a condition (e.g., a disease of the eye or symptoms associated therewith), or the amelioration of one or more symptoms resulting from the administration of one or more therapies (including, but not limited to, the administration of one or more prophylactic or therapeutic agents).

“Administering” or “administration of a substance, a compound or an agent to a subject can be carried out using one of a variety of methods known to those skilled in the art. For example, a compound or an agent can be administered intravitreally or subretinally or systemically. In particular examples, the compound or agent is administered intravitreally. In some examples, administration may be local. In other examples, administration may be systemic. Administering can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient.

Each example described herein may be used individually or in combination with any other examples described herein.

In some examples, the therapeutic peptides described herein are synthetically engineered mimetics relating to the structure or function of one or more endogenous molecules. For example, endogenous apolipoproteins involved in lipid transport and processing mechanisms are responsible for the solubilization and trafficking of lipid molecules for cellular uptake or efflux. In particular, apolipoproteins have a direct involvement in cholesterol homeostasis in RPE relating to the uptake or efflux of lipids such as cholesterol.

The therapeutic compositions described herein may include one or more therapeutic peptides having lipid solubilizing and lipid efflux activity; these therapeutic compositions may have minimal cytotoxicity.

As mentioned, the therapeutic peptides described herein may be short, amphipathic polypeptides (e.g., 80 or fewer amino acids, 75 or fewer amino acids, 70 or fewer amino acids, 65 or fewer amino acids, 60 or fewer amino acids, 55 or fewer amino acids, 50 or fewer amino acids, 45 or fewer amino acids, 40 or fewer amino acids, fewer than 35 amino acids, fewer than 30 amino acids, etc.) that may bind one or more cholesterol efflux transporter proteins such as ABCA1 or other transporters) and promote lipid efflux out of cells. Accordingly, the therapeutic peptide mimetics described herein may have homologous structure to endogenous apolipoprotein A (e.g., ApoA1) function may confer therapeutic benefit to a condition based on dysregulation of lipid efflux mechanisms for the prevention of lipid accumulation or increased drusen formation.

The therapeutic compositions described herein may include one or more therapeutic peptides having lipid solubilizing and lipid import activity; these therapeutic compositions may have minimal cytotoxicity.

As mentioned, the therapeutic peptides described herein may be short, amphipathic that may bind LDLR in a lipid-dependent manner and transport lipids into cells via LDLR receptor. These therapeutic peptides may overcome lipid transport deficiency present in ApoE2 carriers, such as the ApoE2 variant which has defective LDLR binding activity. These therapeutic peptides preserve lipid-dependent LDLR binding and lipid transport activity in a smaller peptide. Accordingly, the therapeutic peptide mimetics described herein may have homologous structure to endogenous apolipoprotein E (e.g., ApoE) function may confer therapeutic benefit to a condition based on dysregulation of lipid import mechanisms for the prevention of lipid accumulation or increased drusen formation.

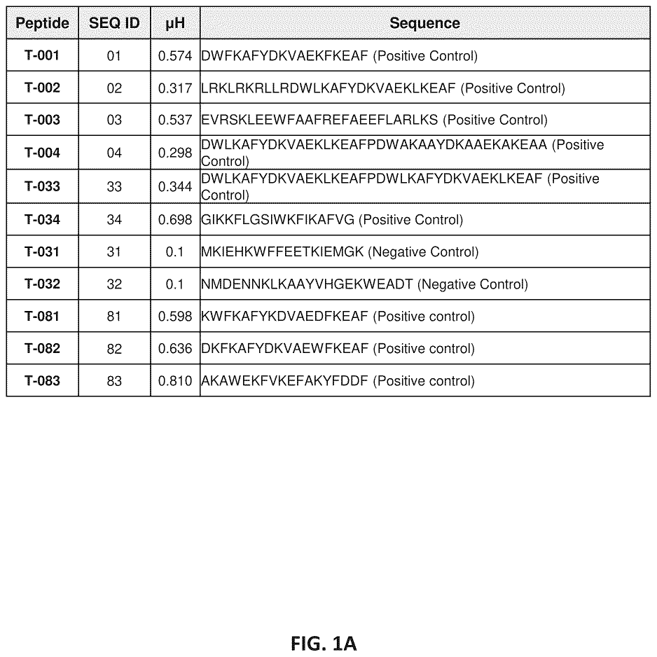

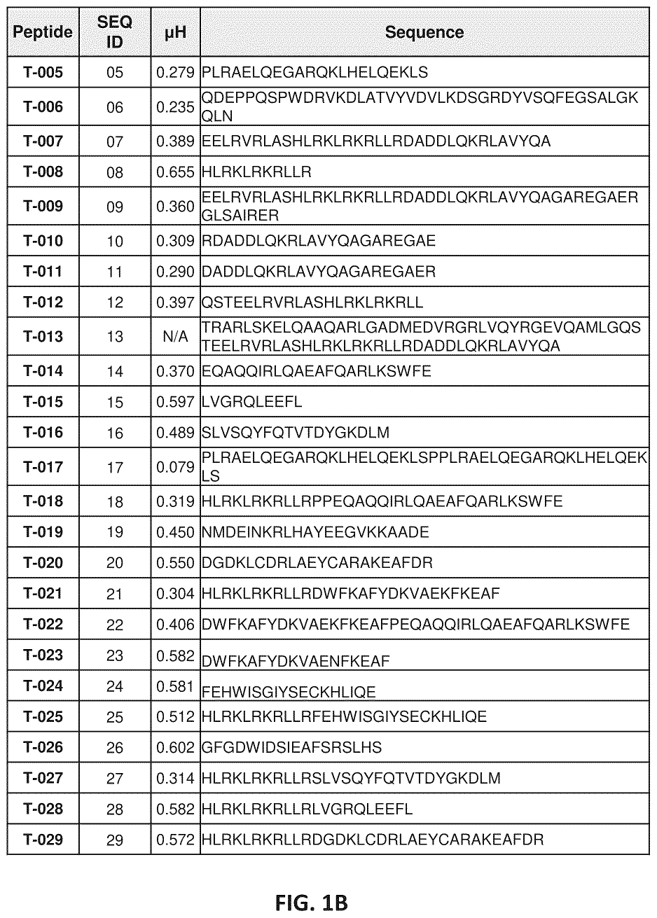

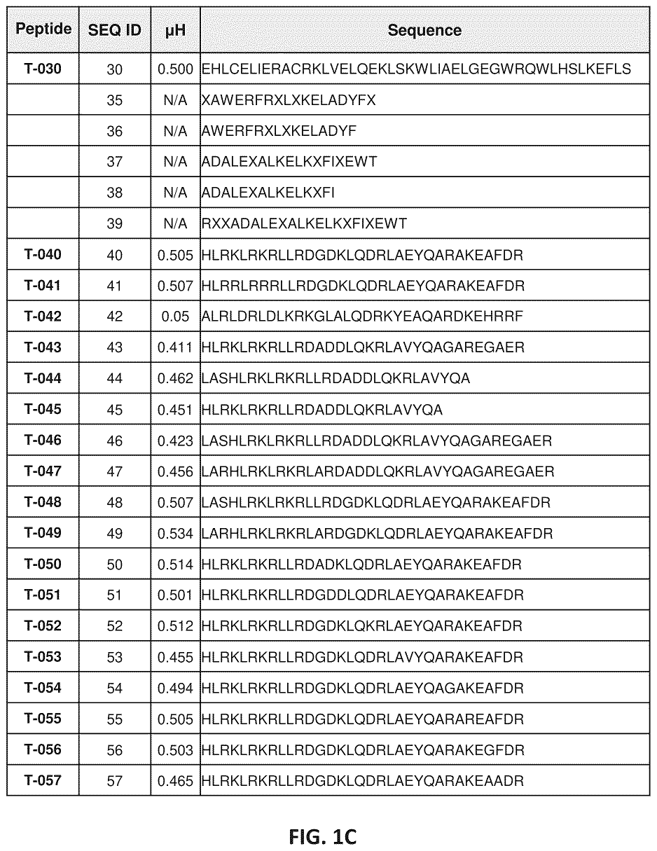

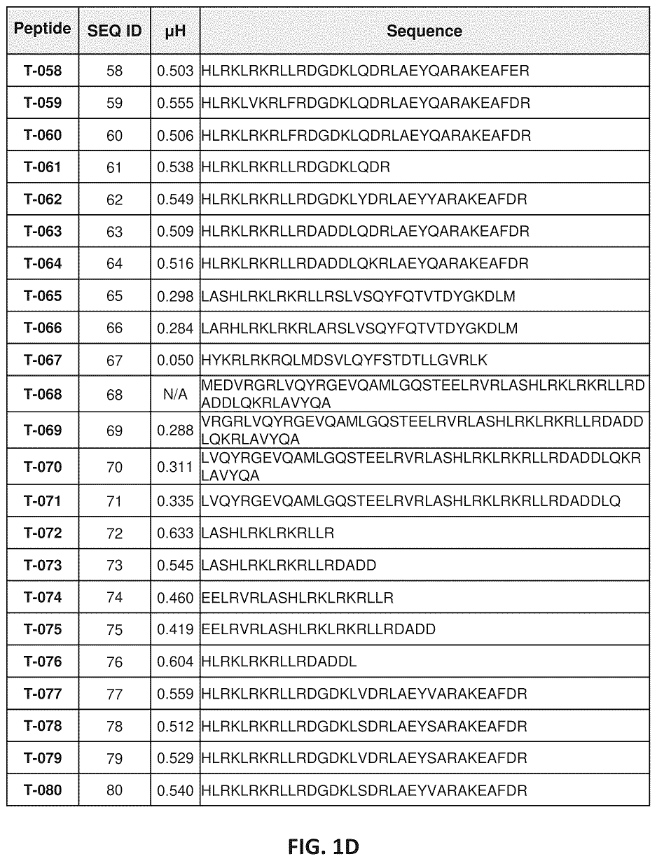

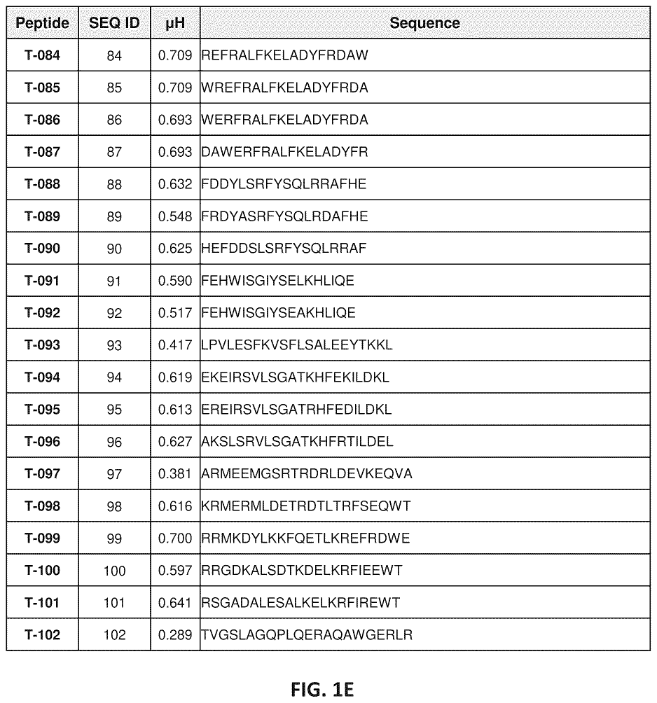

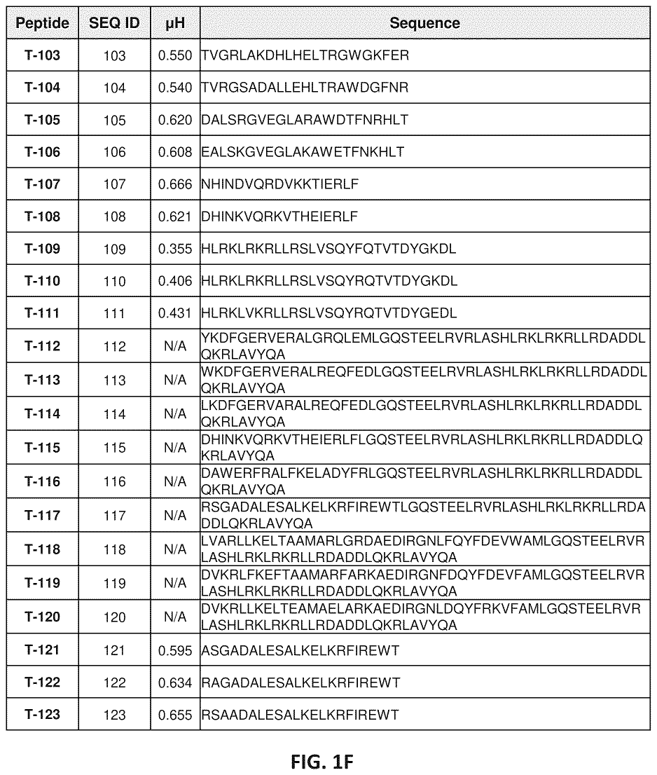

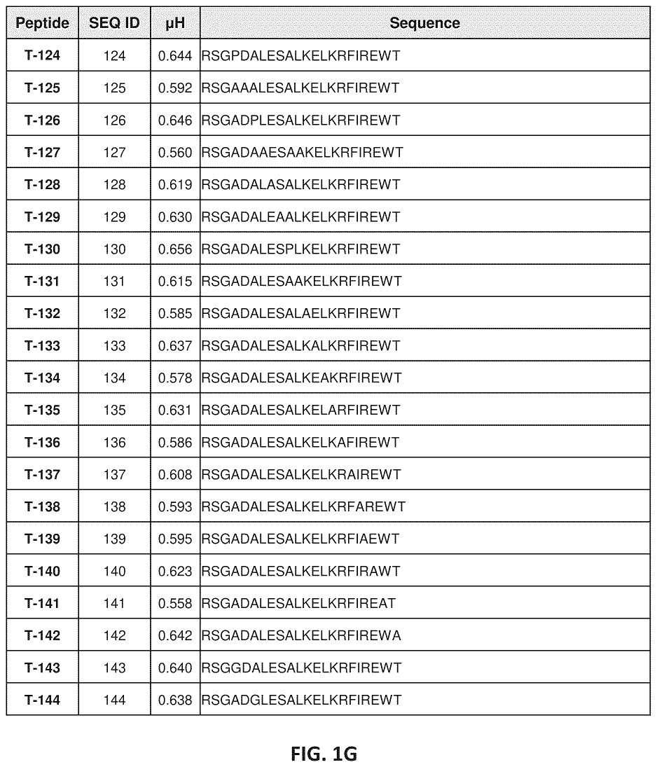

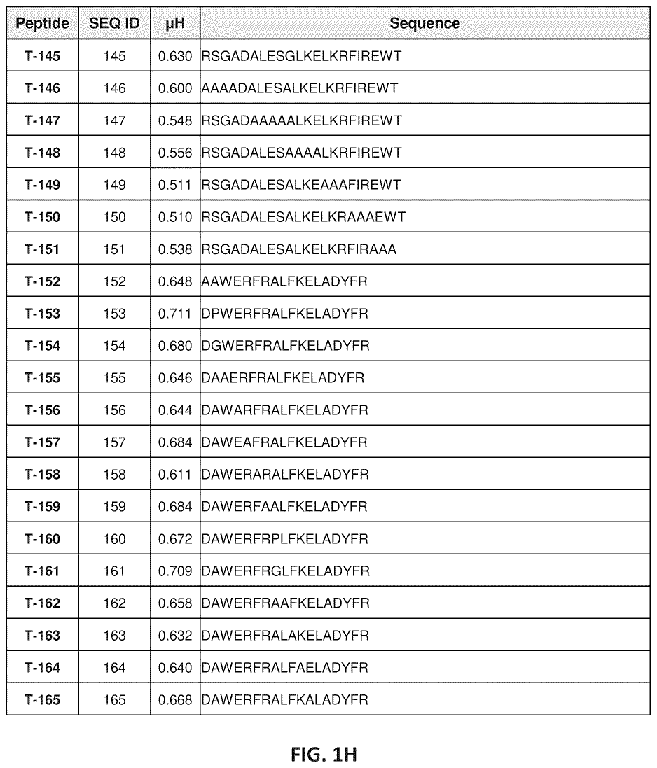

FIGS. 1A-1I illustrates peptides including control (T-001 to T-004, and T-031 to T-034) and therapeutic peptides (T-005 to T-030, and T-35 to T-178 corresponding to SEQ ID NOS: 5-30 and 35-178, respectively). These peptides, or modified versions of these peptides, may be used as therapeutic peptides as described herein. The tables shown in FIGS. 1A-1I also include the helical hydrophobic moment (μH), which is a measure of the amphiphilicity of the helix of the peptide.

As mentioned, any of the peptides, T-005 to T-030, T-35 to T-080, and T-084 to T-178, and related peptides (e.g., peptides having 65% or more homology) may be used therapeutically as described herein. Peptides that are at least 65% (in some examples, at least 75% homologous, at least 80% homologous, at least 85% homologous, at least 90% homologous) with any of the peptides of T-005 to T-030, T-35 to T-080, and T-084 to T-178 may refer to homologous peptides in which the different amino acid residues are conservative substitutions. In any of these examples homologous peptides may refer to peptides in which the amino acids that differ from the peptides of T-005 to T-030, T-035 to T-080, and T-084 to T-178 are conservative hydrophobicity substitutions and/or conservative size substitutions and/or conservative charge substitutions. In any of these examples the conservative substitution (and/or the conservative hydrophobicity substitutions and/or the conservative size substitutions and/or the conservative charge substitutions) maintain a helical structure as described herein.

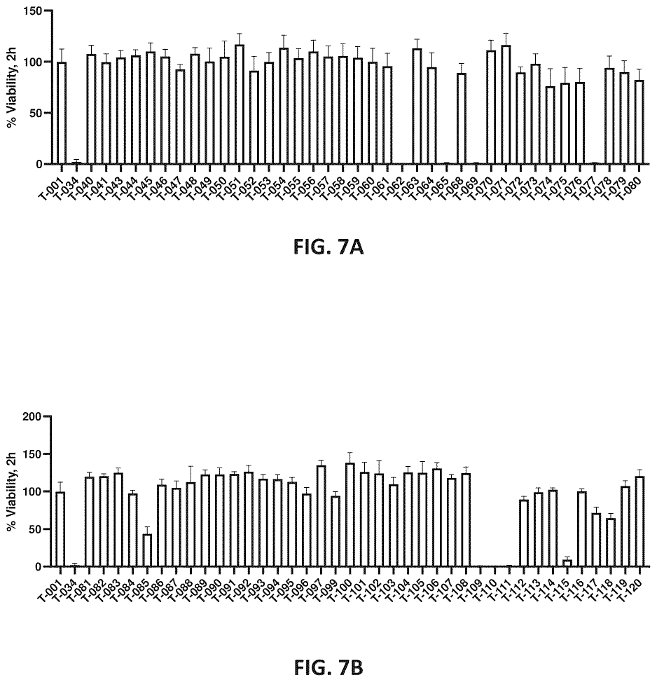

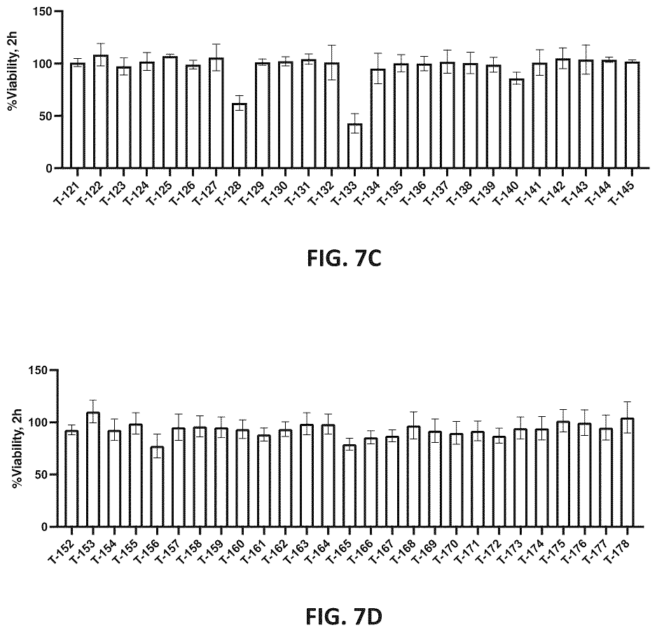

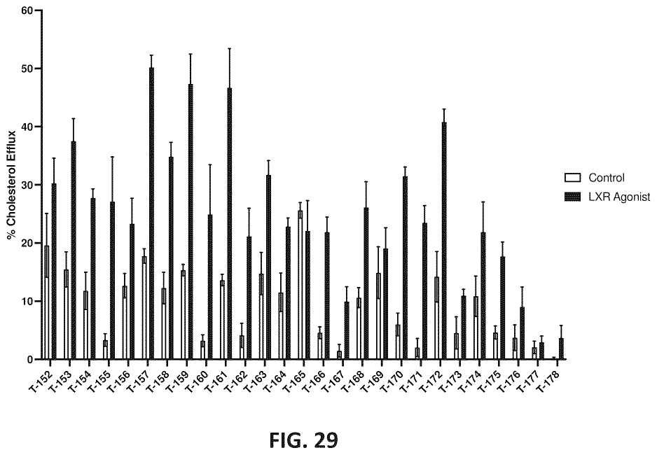

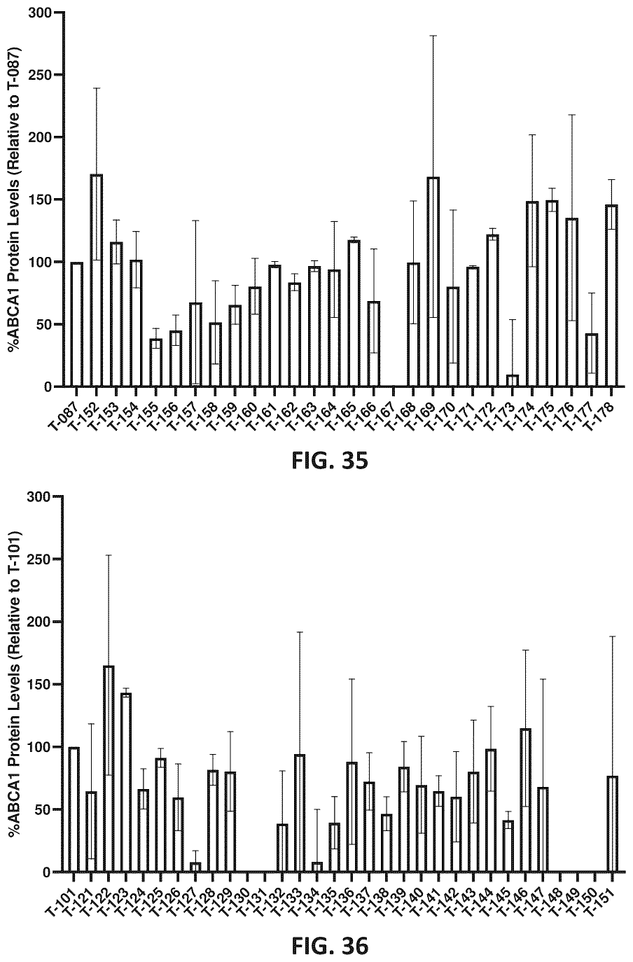

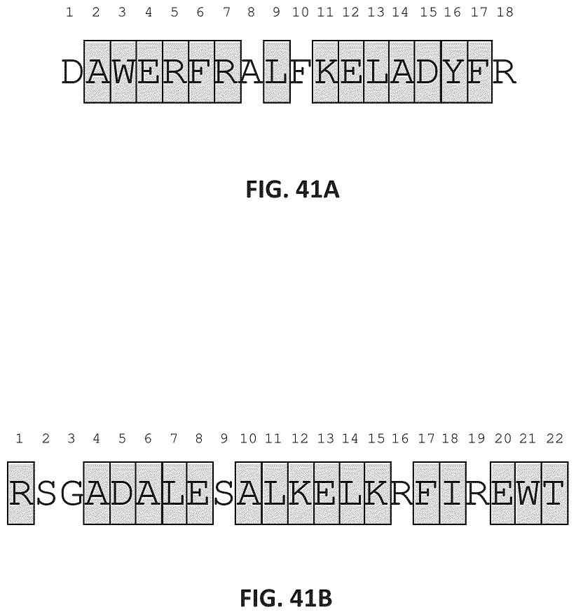

In particular, one example, the T-087 (SEQ ID NO.: 87) peptide, or a peptide that is at least 65% homologous to the T-087 peptide, may be used therapeutically as described herein. For example, FIG. 41A shows the sequence of one family of homologous peptides based on the T-087 peptide. In FIG. 41A, the boxed amino acids indicate residues that contribute to the desired therapeutic activities of the peptide, including safety, cholesterol efflux capacity, and ABCA1 stabilization capacity. These residues were determined by testing derivatives of T-087 created through alanine scanning (single and triple) mutagenesis in functional assays as described in FIG. 7D, FIG. 29 and FIG. 35. The peptides T-152 to T-178 illustrate some of the example peptides corresponding to variants of T-087. In FIG. 41A the unboxed amino acids may correspond to any amino acid. In some examples the boxed amino acid corresponds to a conservative substitution (e.g., using Glycine (G) in the second position, Phenylalanine (F) in the third position, Aspartic acid (D) in the fourth position, Lysine (K) in the fifth position, Leucine (L) in the sixth position, Arginine (R) in the seventh position, Isoleucine (I) in the ninth position, Arginine (R) in the eleventh position, Aspartic acid (D) in the twelfth position, Valine (V) in the thirteenth position, Glycine (G) in the fourteenth position, Glutamic acid (E) in the fifteenth position, Tryptophan (W) in the sixteenth position, and Tyrosine (Y) in the seventeenth position), a conservative hydrophobicity substitution (e.g. Threonine (T) in the second position, Aspartic acid (D) in the fourth position, Aspartic acid (D) in the fifth position, Isoleucine (I) in the sixth position, Aspartic acid (D) in the seventh position, Phenylalanine (F) in the ninth position, Aspartic acid (D) in the eleventh position, Asparagine (N) in the twelfth position, Phenylalanine (F) in the thirteenth position, Threonine (T) in the fourteenth position, Lysine (K) in the fifteenth position, Proline (P) in the sixteenth position, and Leucine (L) in the seventeenth position), or a conservative size substitution (e.g. Glycine (G) or Serine(S) in the second position, Tyrosine (Y) in the third position, Glutamine (Q) or Methionine (M) in the fourth position, Phenylalanine (F) or Tyrosine (Y) in the fifth position, Histidine (H) or Arginine (R) in the sixth position, Phenylalanine (F) or Tyrosine (Y) in the seventh position, Cysteine (C) or Isoleucine (I) in the ninth position, Aspartic acid (D) or Glutamine (Q) in the eleventh position, Glutamine (Q) or Methionine (M) in the twelfth position, Cysteine (C) or Isoleucine (I) in the thirteenth position, Glycine (G) or Serine(S) in the fourteenth position, Asparagine (N) or Lysine (K) in the fifteenth position, Arginine (R) or Tryptophan (W) in the sixteenth position, and Histidine (H) or Arginine (R) in the seventeenth position).

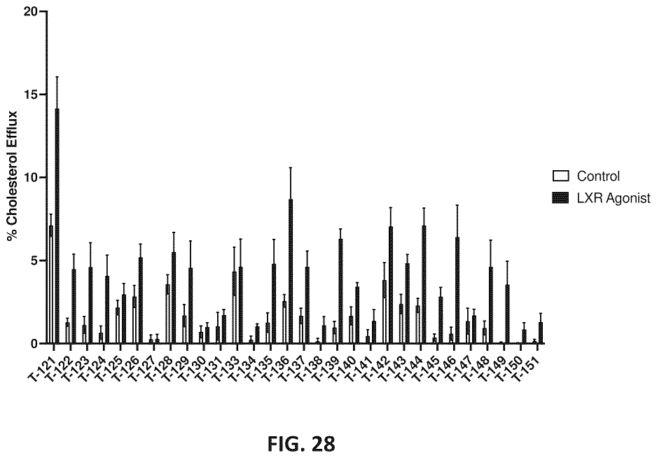

In another example, the T-101 (SEQ ID NO.: 101) peptide, or a peptide that is at least 65% homologous to the T-101 peptide, may be used therapeutically as described herein. For example, FIG. 41B shows the sequence of one family of homologous peptides based on the T-101 peptide. In FIG. 41B, the boxed amino acids indicate residues that contribute to the desired therapeutic activities of the peptide, including safety, cholesterol efflux capacity, and ABCA1 stabilization capacity. These residues were determined by testing derivatives of T-101 created through alanine scanning mutagenesis (single and triple) in functional assays as described in FIG. 7C, FIG. 28 and FIG. 36. The peptides T-121 to T-151 illustrate some of the example peptides corresponding to variants of T-101. In FIG. 41B the unboxed amino acid may correspond to any amino acid. In some examples the boxed amino acid corresponds to a conservative substation (e.g. using Lysine (K) in the first position, Glycine (G) in the fourth position, Glutamic acid (E) in the fifth position, Glycine (G) in the sixth position, Valine (V) in the seventh position, Aspartic acid (D) in the eighth position, Glycine (G) in the tenth position, Isoleucine (I) in the eleventh position, Arginine (R) in the twelfth position, Aspartic acid (D) in the thirteenth position, Valine (V) in the fourteenth position, Arginine (R) in the fifteenth position, Tryptophan (W) in the seventeenth position, Leucine (L) in the eighteenth position, Aspartic acid (D) in the twentieth position, Phenylalanine (F) in the twenty-first position, and Serine(S) in the twenty-second position), a conservative hydrophobicity substitution (e.g. Aspartic acid (D) in the first position, Threonine (T) in the fourth position, Lysine (K) or Arginine (R) in the fifth position, Histidine (H) in the sixth position, Phenylalanine (F) in the seventh position, Asparagine (N) in the eighth position, Threonine (T) in the tenth position, Isoleucine (I) in the eleventh position, Aspartic acid (D) in the twelfth position, Asparagine (N) in the thirteenth position, Cysteine (C) in the fourteenth position, Aspartic acid (D) in the fifteenth position, Leucine (L) in the seventeenth position, Phenylalanine (F) in the eighteenth position, Asparagine (N) in the twentieth position, and Histidine (H) in the twenty-second position), or a conservative size substitution (e.g. Phenylalanine (F) or Tyrosine (Y) in the first position, Glycine (G) or Serine(S) in the fourth position, Asparagine (N) or Lysine (K) in the fifth position, Glycine (G) or Serine(S) in the sixth position, Cysteine (C) or Isoleucine (I) in the seventh position, Glutamine (Q) or Methionine (M) in the eighth position, Glycine (G) or Serine(S) in the tenth position, Cysteine (C) or Isoleucine (I) in the eleventh position, Aspartic acid (D) or Glutamine (Q) in the twelfth position, Glutamine (Q) or Methionine (M) in the thirteenth position, Cysteine (C) or Isoleucine (I) in the fourteenth position, Aspartic acid (D) or Glutamine (Q) in the fifteenth position, Histidine (H) or Arginine (R) in the seventeenth position, Leucine (L) or Asparagine (N) in the eighteenth position, Glutamine (Q) or Methionine (M) in the twentieth position, Tyrosine (Y) in the twenty-first position, and Valine (V) or Cysteine (C) in the twenty-second position).

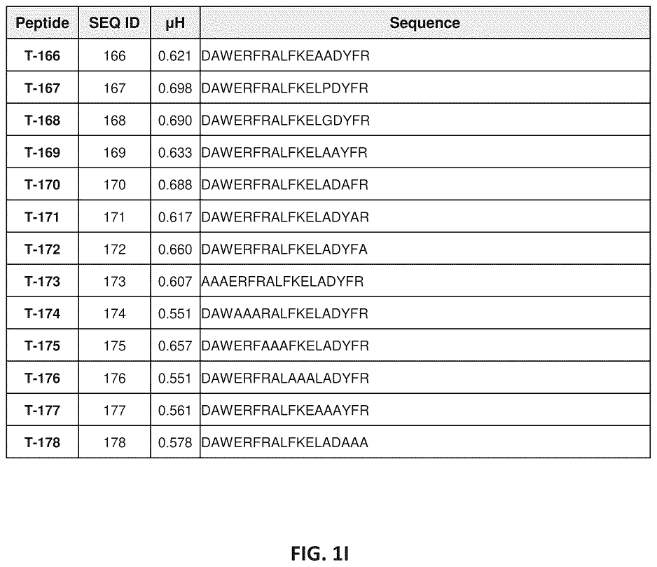

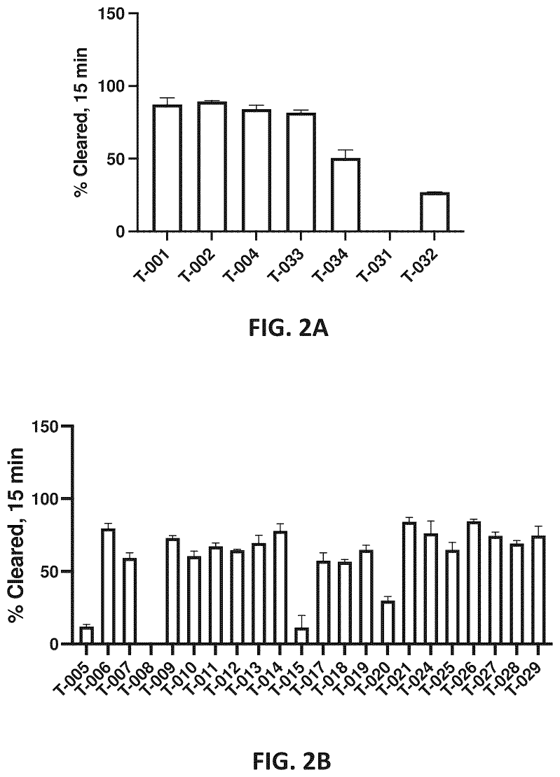

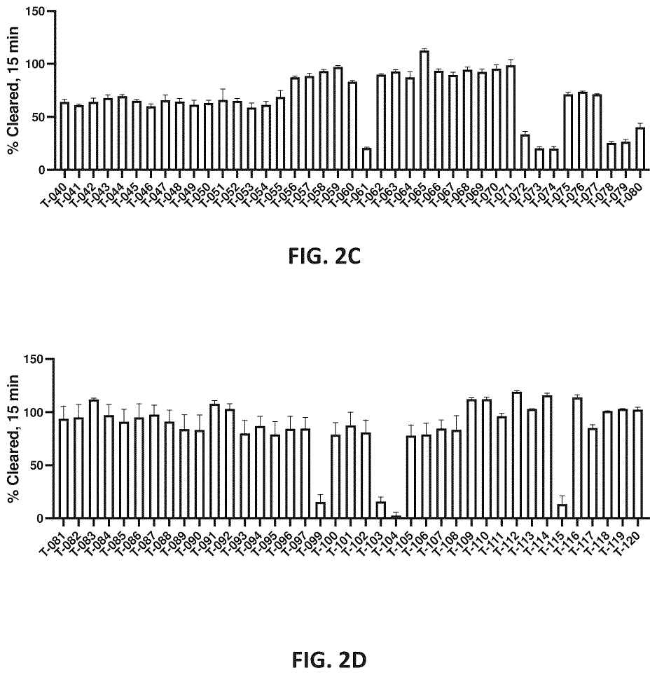

The safety and functional activities of the peptides described herein are illustrated in the figures and data provided herein. For example, FIGS. 2A-2D illustrate lipid solubilization activity, measured by reducing turbidity of 1,2-dimyristoyl-sn-glycero-3-phosphocholine (DMPC) liposome solutions, for each of the peptides shown. In this example, peptide stocks were diluted to 200 μM in H2O (a 2× solution) and 50 μL of peptide solution was transferred in triplicate into 96-well plates. A one mM solution of 1,2-dimyristoyl-sn-glycero-3-phosphocholine (DMPC) in H₂O was prepared, and using a multichannel pipet, 50 μL DMPC solution was rapidly added to the plate. Peptide and lipid solutions were incubated at room temperature for 15 minutes with a final concentration of 100 μM peptides and 0.5 mM DMPC. After 15 minutes, absorbance was read on a plate reader at 405 nm. H2O blanks were used for background correction, and 100% lipid clearance was determined using the absorbance of DMPC incubated in 1% Triton-X-100. Data are presented as mean+SD over two runs. Nearly all tested peptides cleared over 50% of lipid over the course of the assay.

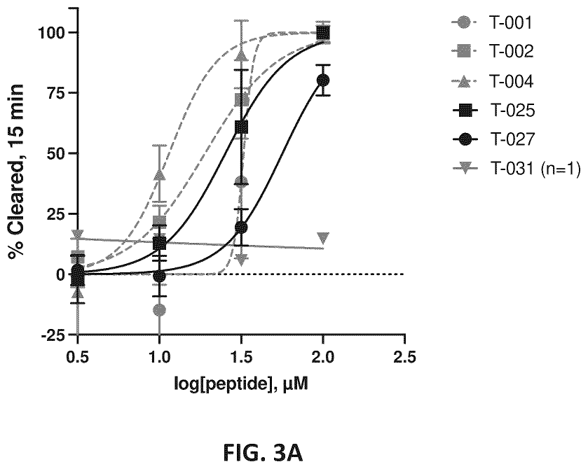

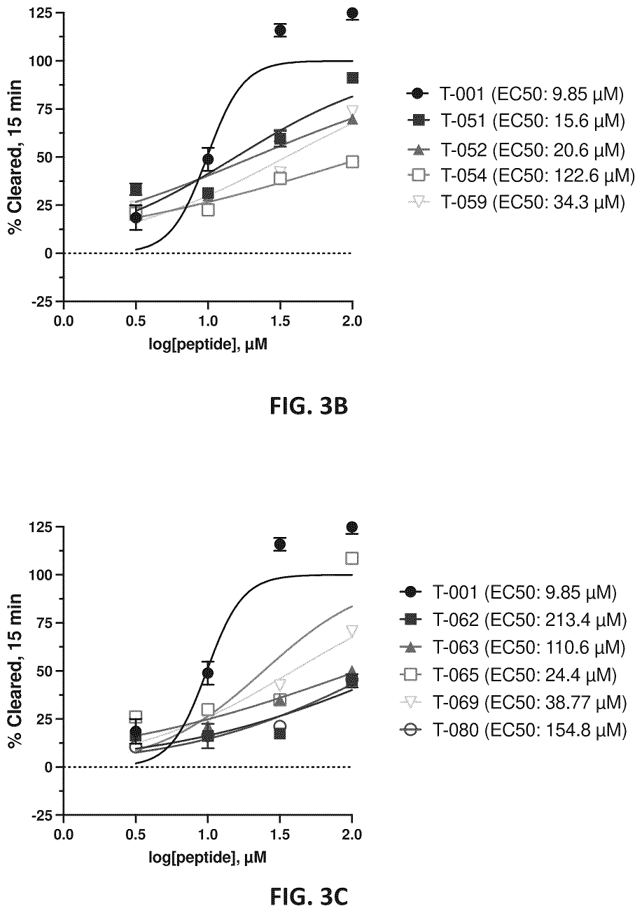

Eleven of the test peptides (T-025, T-027, T-051, T-052, T-054, T-059, T-062, T-063, T-065, T-069 and T-080) were examined in greater detail, as shown in FIGS. 3A-3C. In these experiments, DMPC solubility titration curves were generated. Peptide stocks were diluted to 200 μM in H2O (a 2× solution) and serially diluted in H2O over 4 concentrations, then 50 μL of peptide solutions were distributed in triplicate into 96-well plates. A 1 mM solution of 1,2-dimyristoyl-sn-glycero-3-phosphocholine (DMPC) in H2O was prepared, and using a multichannel pipet, 50 μL DMPC solution was rapidly added to the plate. Peptide and lipid solutions were incubated at room temperature for 15 minutes with final concentrations of 3.16-100 μM peptides and 0.5 mM DMPC. After 15 minutes, absorbance was read on a plate reader at 405 nm. H2O blanks were used for background correction, and 100% lipid clearance was determined using the absorbance of DMPC incubated in 1% Triton-X-100. Data are presented as mean+SD over two runs. Non-linear regression was used to calculate EC50 values. Test peptide EC50 values in the assay were (T-025, 25.33 μM; T-027, 56.55 μM; T-051, 15.6 μM; T-052, 20.6 μM; T-054, 122.6 μM; T-062, 213.4 μM; T-063, 110.6 μM; T-065, 24.4 μM; T-069, 38.77 μM; T-080, 154.8 μM).

From these results shown in FIGS. 2A-2D and 3A-3C, a peptide is likely to be particularly therapeutically useful if more than 50% was cleared at 15 min at 100 μM (and particularly if there was activity at 10 μM). Many of the non-control peptides had significant activity. See, e.g., T-006, T-007, T-009, T-010, T-011, T-012, T-013, T-014, T-024, T-026, T-030, T-018, T-019, T-021, T-025, T-027, T-028, T-029, T-086, T-087, T-101, T-112, T-114, T-116. Some of these peptides (T-005, T-008, T-015, T-017, T-020, and T-099) did not have significant solubilizing activity.

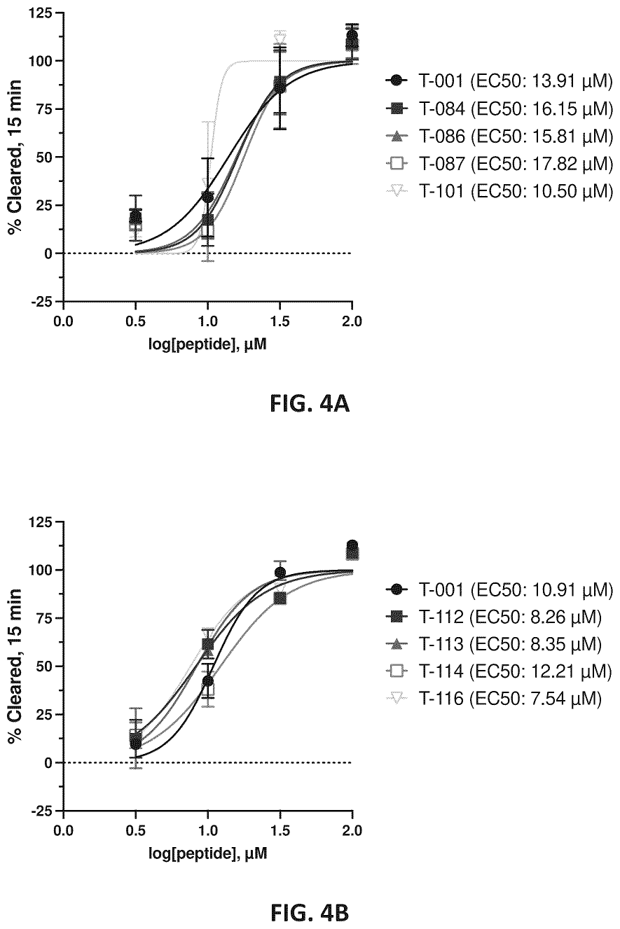

FIGS. 4A-4B illustrate a screening assay based on DMPC solubility, measured by reducing turbidity of DMPC liposome solutions, for a subset of the peptides in FIGS. 1B-1I. FIGS. 4A-4B illustrate DMPC solubility titration curves. Peptide stocks were diluted to 200 μM in H₂O (a 2× solution) and serially diluted in H₂O over 4 concentrations, then 50 μL of peptide solutions were distributed in triplicate into 96-well plates. A 2 mM solution of 1,2-dimyristoyl-sn-glycero-3-phosphocholine (DMPC) in H₂O was prepared, and using a multichannel pipet, 50 μL DMPC solution was rapidly added to the plate. Peptide and lipid solutions were incubated at room temperature for 15 minutes with final concentrations of 3.16-100 μM peptides and 1 mM DMPC. After 15 minutes, absorbance was read on a plate reader at 405 nm. H₂O blanks were used for background correction, and 100% lipid clearance was determined using the absorbance of DMPC incubated in 1% Triton-X-100. Data are presented as mean+SD over two runs. Non-linear regression was used to calculate EC50 values. Test peptide EC50 values in the assay were (T-084, 16.15 μM; T-086, 15.81 μM; T-087, 17.82 μM; T-101, 10.50 μM; T-112, 8.26 μM; T-113, 8.35 μM; T-114, 12.21 μM; T-116, 7.54 μM)

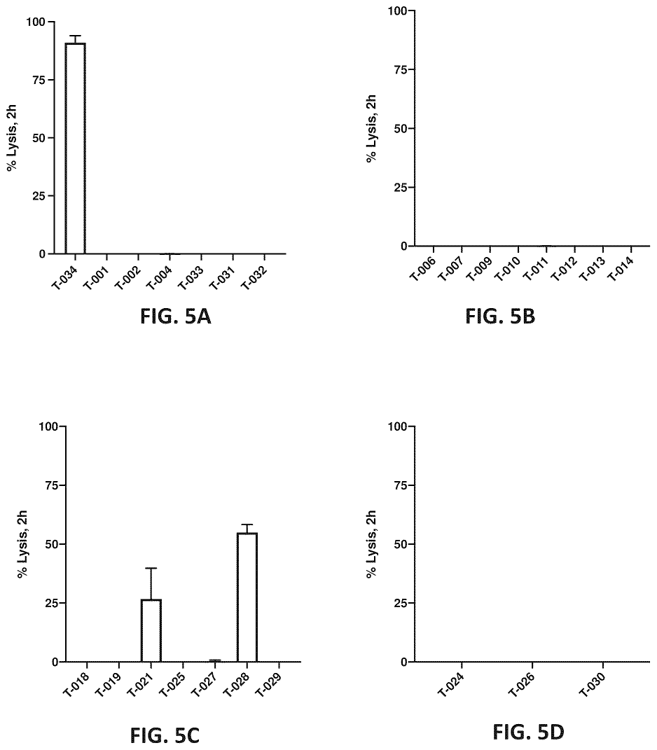

Select test peptides were evaluated further as shown in FIGS. 5A-5D. These figures illustrate ARPE-19 cell lysis. ARPE-19 cells were grown in 96-well plates to 75-85% confluence. Peptides at 10 mM were diluted in serum-free DMEM:F12 media into a 20 μM working solution (a 2× solution). Sytox Green was prepared in serum-free DMEM:F12 at a 1 μM working solution (a 2× solution). Equivalent volumes of the peptide and Sytox Green solutions were added to empty 96-well plates and mixed gently. Final peptide concentration was 10 μM. ARPE-19 cells (˜80% confluent in 96-well plates) were washed with DPBS and then incubated in 100 μL of peptide/Sytox Green mixture for 2 h at 37° C. Cells were fixed in 4% paraformaldehyde solution in DPBS and then incubated in 10 μM Hoechst solution for 10 min, washed, and imaged on a Yokogawa CQ1 high-content imager. CellPathfinder software was used to count live/dead cells in 4 fields of view at 20× magnification. Percent (%) lysis was calculated by dividing the number of Sytox Green+ nuclei by the total number of Hoechst+ nuclei. In this assay, test peptides did not induce cell lysis, with the exception of T-021, T-028 and T-034.

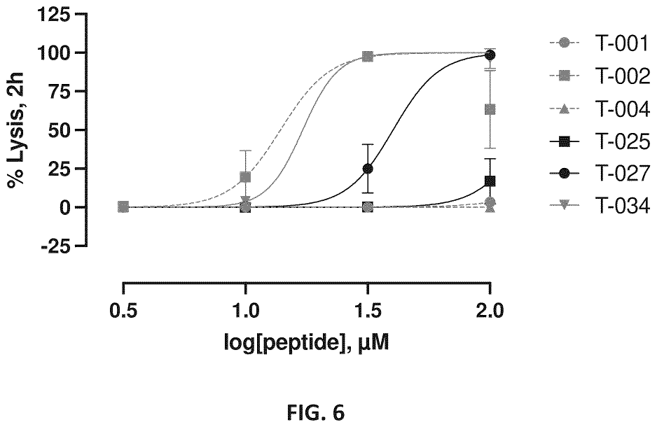

FIG. 6 shows ARPE-19 cell lysis titration curves for a subset of the peptides of FIGS. 1B-1I. ARPE-19 cells were grown in 96-well plates to 75-85% confluence. Peptides at 10 mM were diluted in serum-free DMEM: F12 media to solutions between 6.32-200 M. Sytox Green was prepared in serum-free DMEM: F12 media at a 1 μM working solution. Equivalent volumes of the peptide and Sytox Green solutions were added to empty 96-well plates and mixed gently. Final peptide concentration was 3.16-100 μM. ARPE-19 cells (˜80% confluent in 96-well plates) were washed with DPBS and then incubated in 100 μL of peptide/Sytox Green mixture for 2 h at 37° C. Cells were fixed in 4% paraformaldehyde solution in DPBS and then incubated in 10 μM Hoechst solution for 10 min, washed, and imaged on a Yokogawa CQ1 high-content imager. CellPathfinder software was used to count live/dead cells in 4 fields of view at 20× magnification. Percent (%) lysis was calculated by dividing the number of Sytox Green+ nuclei by the total number of Hoechst+ nuclei. Non-linear regression was used to calculate EC50 values. Test peptide EC50 values in the assay were (T-025, 144.1 μM; T-027, 40.14 μM)

FIGS. 7A-7D illustrate the results of ARPE-19 cell viability assays on the peptides described herein. ARPE-19 cells were seeded in 96-well plates at 25,000 cells/well and allowed to adhere to the plate surface for at least 24 hours. Peptides were diluted in serum-free DMEM: F12 to a final concentration of 100 μM. Cells were washed, then incubated in 100 μM peptide solution for 1.5 hours at 37° C. Plates were brought to room temperature for 30 minutes, then washed with DPBS. Cells were incubated in a 1:1 mixture of DPBS and Cell Titer Glo 2.0 Reagent for 10 minutes at room temperature, then cell luminescence was read on a plate reader. Viability values were background corrected, and 0% viability is determined by luminescence values of cells treated with 1% Triton-X-100. Data are presented as mean+SD over two separate runs. Treatment with test peptides resulted in no substantial change in viability over the course of the assay, with the exception of test peptides T-062, T-065, T-069, T-077, T-085, T-109, T-110, T-111, and T-115.

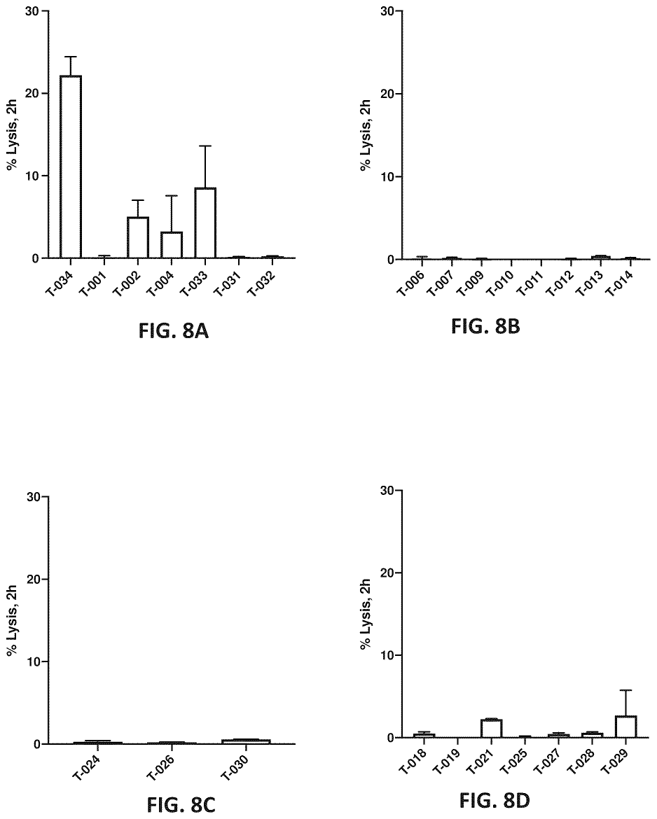

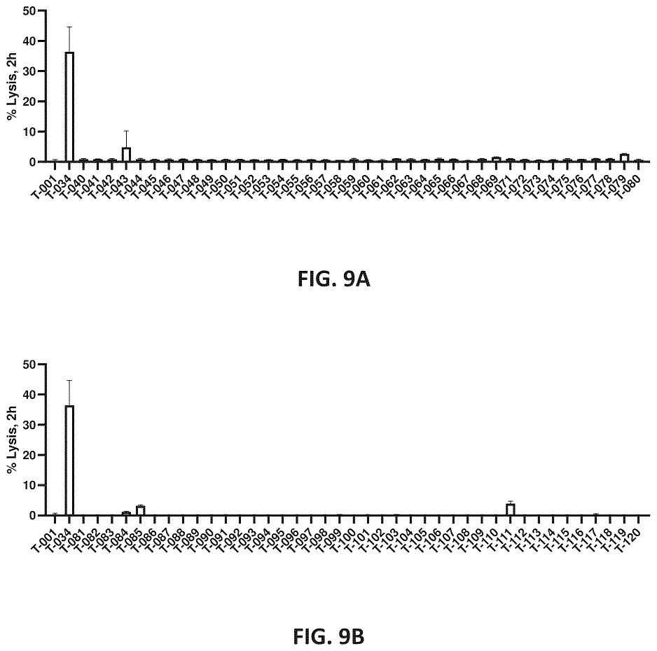

Similar results were seen with human red blood cells (RBCs) in a lysis assay, as shown in FIGS. 8A-8D and 9A-9B. FIGS. 8A-8D show the results of human red blood cell (hRBC) lysis assays on a subset of the peptides. Human erythrocytes (hRBCs; washed, suspended at 25% dilution of cells in Alsevers solution) were added in duplicate 50 μL aliquots to 96-well plates containing 50 μL of 40 μM peptide solutions. Plates were incubated for 2 h at 37° C., then hRBCs were pelleted at 500×g for 5 min. Fifty μL of supernatant was collected and transferred to fresh plates containing 50 μL PBS per well and absorbance was read on a plate reader at 560 nm to determine lysis. 100% lysis reflects wells treated with 0.1% Triton-X-100. Data are presented as mean+SD for two runs. FIGS. 9A-9B are graphs summarizing the hRBC lysis assay for more of the peptides. Test peptides did not induce greater than 10% hRBC lysis in the assay.

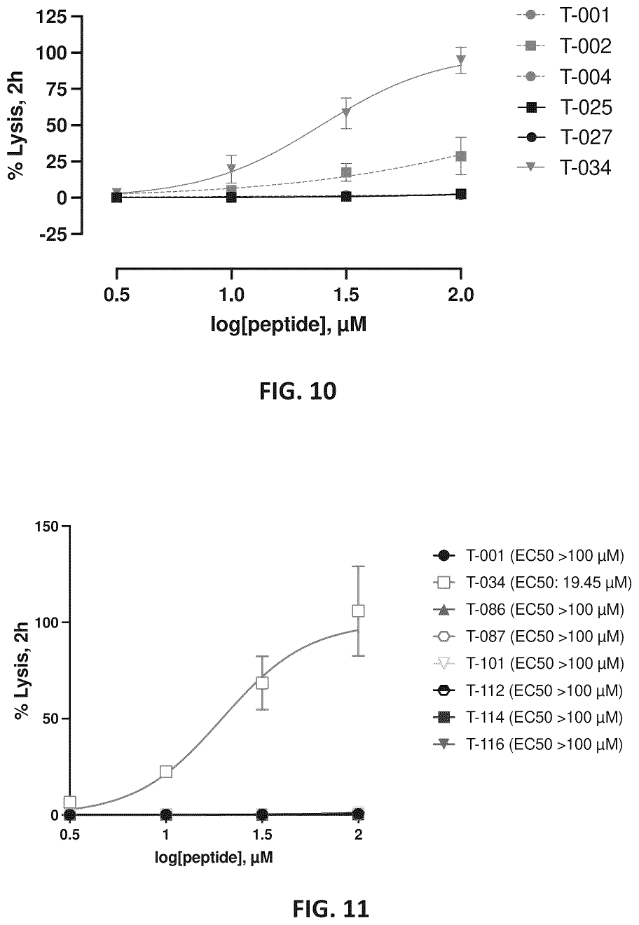

FIGS. 10 and 11 illustrate human red blood cell (hRBC) lysis titration curves. Human erythrocytes (hRBCs; washed, suspended at 25% dilution of cells in Alsevers solution) were added in duplicate 50 μL aliquots to 96-well plates containing 50 μL of 6.32-200 μM peptide solutions (final peptide concentrations were 3.16-100 μM). Plates were incubated for 2 h at 37° C., then hRBCs were pelleted at 500×g for 5 min. Fifty μL of supernatant was collected and transferred to fresh plates containing 50 μL PBS per well and absorbance was read on a plate reader at 560 nm to determine lysis. 100% lysis reflects wells treated with 0.1% Triton-X-100. Data are presented as mean+SD for two runs. Non-linear regression was used to calculate EC50 values. Test peptide EC50 values in this assay were (T-025, >100 μM; T-027, >100 μM; T-086, >100μ; T-087, >100 μM; T-101, >100μ; T-112, >100 μM; T-114, >100μ; T-116, >100 μM)

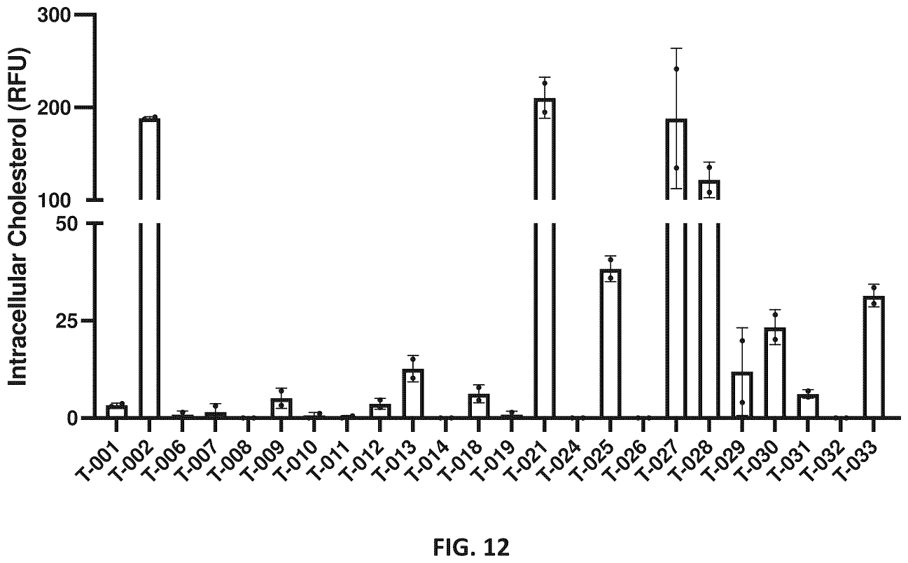

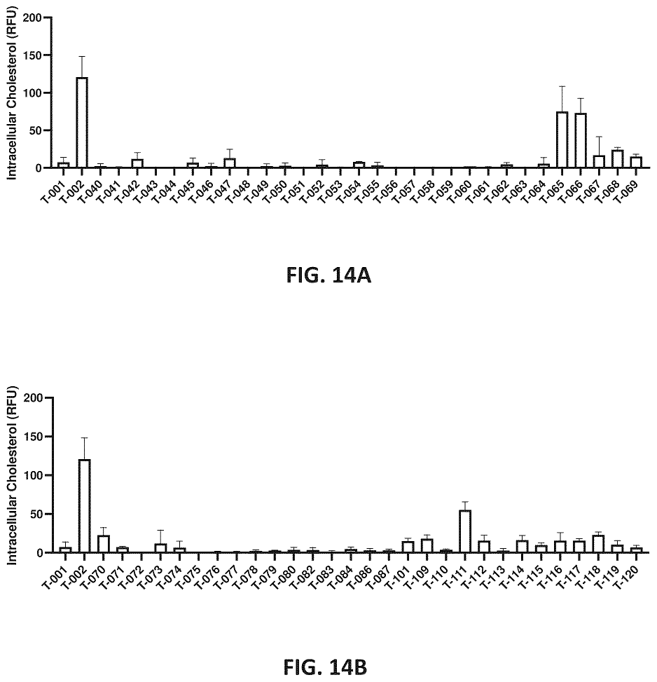

FIG. 12 and FIGS. 14A-14B illustrate the results of ARPE-19 cholesterol import testing. ARPE-19 cells were seeded on 96-well plates at 25,000 cells per well and grown to confluence. A 2× solution of 500 μM DMPC and 50 μM BODIPY-Cholesterol were prepared in serum free DMEM: F12. Peptides were diluted to 20 μM in serum free DMEM: F12 (a 2× solution). Peptides were added to lipid solution in equal parts (60 μL each) and were incubated for 1 hour at 37° C. ARPE-19 cells were serum starved in serum free DMEM: F12 for 1 hour at 37° C. Peptide: Lipid complexes (100 μL total volume per well) were transferred to ARPE-19 cells for 2 hours at 37° C. Cells were washed, fixed, and intracellular fluorescence was read on Promega GloMax at 475 nm excitation, 500-550 emission. Fluorescence values were background corrected from no treatment conditions. Data are presented as mean+SD for two runs. The results illustrated in FIG. 12 and FIGS. 14A-14B show some of the peptides have cholesterol import activity (corresponding to fluorescence values at least 10 RFU above baseline), for example: T-013, T-018, T-021, T-025, T-027, T-028, T-030, T-042, T-047, T-065, T-066, T-067, T-068, T-069, T-070, T-073, T-101, T-109, T-111, T-112, T-114, T-115, T-116, T-117 T-118, and T-119.

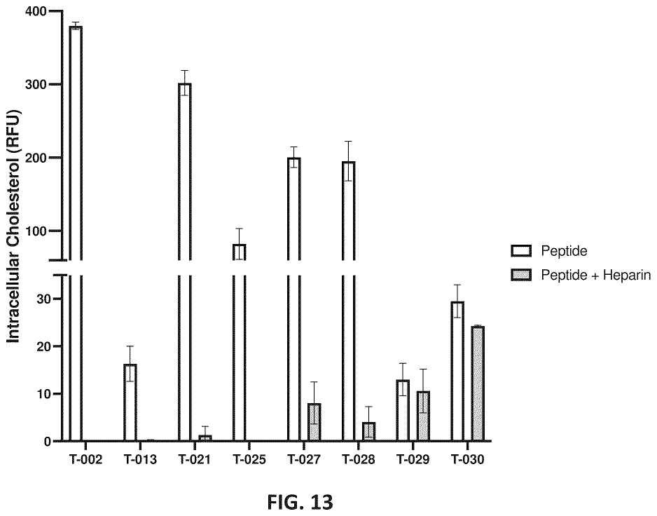

FIG. 13 shows the results of ARPE-19 GAG-dependent cholesterol import assays. ARPE-19 cells were seeded on 96-well plates at 25,000 cells per well and grown to confluence. A 2× solution of 500 μM DMPC and 50 μM BODIPY-Cholesterol with or without 100 g/mL heparin was prepared in serum free DMEM: F12. Peptides were diluted to 20 μM in serum free DMEM: F12 (a 2× solution). Peptides were added to lipid solution in equal parts (60 μL each) and incubated for 1 hour at 37° C. ARPE-19 cells were serum starved in serum free DMEM: F12 for 1 hour at 37° C. Peptide: Lipid complexes (100 μL total volume per well) were transferred to ARPE-19 cells for 2 hours at 37° C. Cells were washed, fixed, and intracellular fluorescence was read on Promega GloMax at 475 nm excitation, 500-550 emission. Fluorescence values were background corrected from no treatment conditions. Data are presented ±SD for 2 runs. Based on these data, test peptides T-013, T-021, T-025, T-027, and T-028 rely on GAG binding for cholesterol import activity.

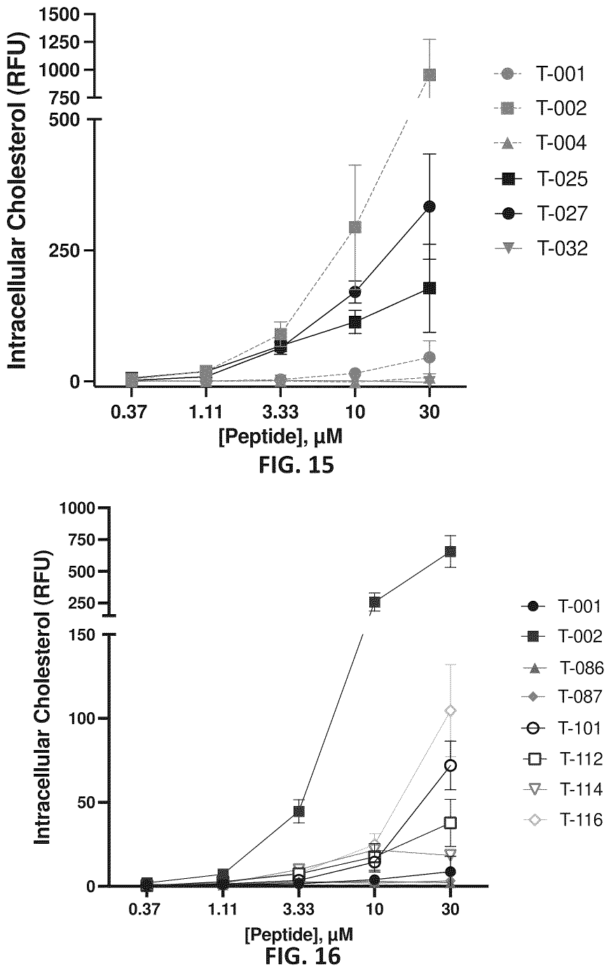

FIG. 15 illustrates ARPE-19 cholesterol import titration curves for a subset of the peptides. ARPE-19 cells were seeded on 96-well plates at 25,000 cells per well and grown to confluence. A 2× solution of 500 μM DMPC and 50 μM BODIPY-Cholesterol was prepared in serum free DMEM: F12. Peptides were diluted to 0.74-60 μM in serum free DMEM: F12 (2× solutions). Peptides were added to lipid solution in equal parts (60 μL each) to create final peptide concentrations of 0.37-30 UM and incubated for 1 hour at 37° C. ARPE-19 cells were serum starved in serum free DMEM: F12 for 1 hour at 37° C. Peptide: Lipid complexes (100 μL total volume per well) were transferred to ARPE-19 cells for 2 hours at 37° C. Cells were washed, fixed, and intracellular fluorescence was read on Promega GloMax at 475 nm excitation, 500-550 emission. Fluorescence values were background corrected from no treatment conditions. Data are presented as mean+SD for three runs. Test peptides T-025 and T-027 demonstrated a concentration-dependent increase in cholesterol import.

FIG. 16 shows ARPE-19 cholesterol import titration curves for another subset of the peptides. ARPE-19 cells were seeded on 96-well plates at 25,000 cells per well and grown to confluence. Peptide: Lipid complexes were created by preparing a solution of serum-free DMEM:F12 containing 30 μM peptide, 750 μM DMPC, and 75 μM BODIPY-Cholesterol. Peptide: Lipid complexes were serially diluted 1:3 for 5 concentrations, and incubated for 1 hour at 37° C. ARPE-19 cells were serum starved in serum free DMEM: F12 for 1 hour at 37° C. Peptide: Lipid complexes (100 μL total volume per well) were transferred to ARPE-19 cells for 2 hours at 37° C. Cells were washed, fixed, and intracellular fluorescence was read on Promega GloMax at 475 nm excitation, 500-550 emission. Fluorescence values were background corrected from no treatment conditions. Data are presented as mean±SD for two runs. Test peptides T-101, T-112, T-114, and T-116 demonstrated concentration-dependent increases in cholesterol import, whereas T-086 and T-087 were not able to increase cholesterol import over a range of concentrations.

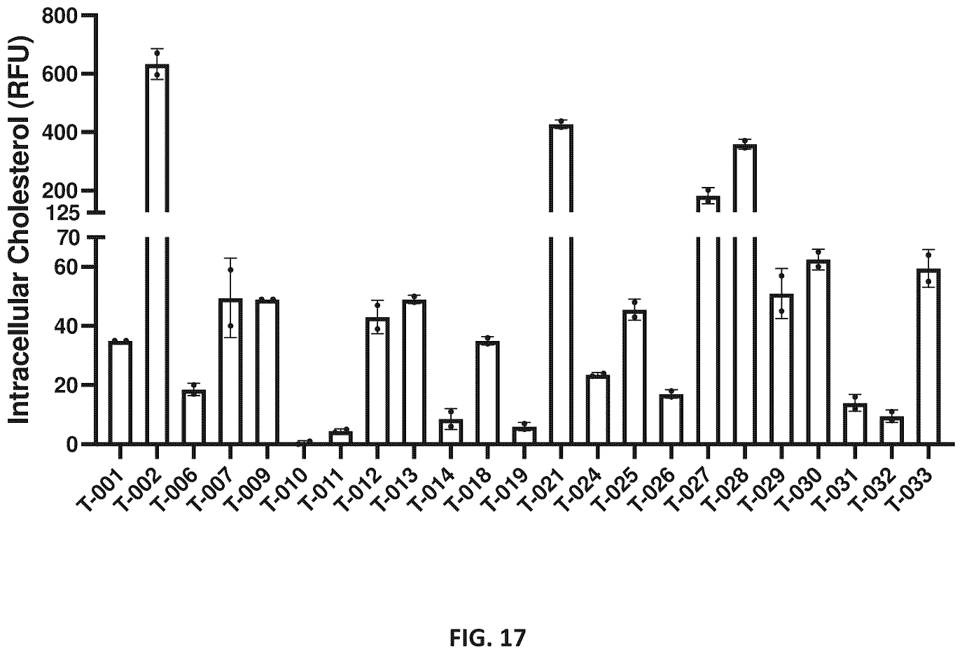

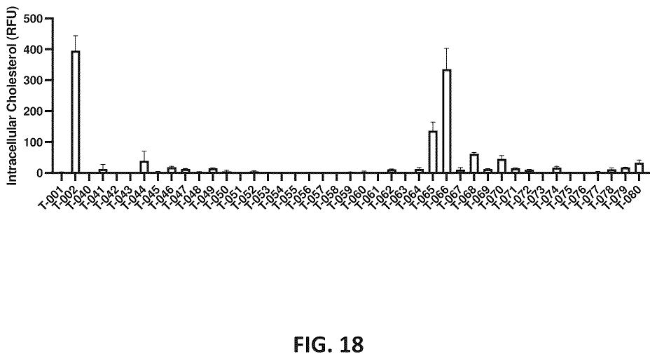

FIGS. 17 and 18 summarize HepG2 cholesterol import screening data for some of the peptides described herein. HepG2 cells were seeded on a 96-well plate at 10,000 cells per well and grown to confluence. A solution of 500 μM DMPC and 50 μM BODIPY-Cholesterol was prepared in serum free EMEM. Peptides were diluted to 20 μM in serum free EMEM (a 2× solution). Peptides were added to lipid solution in equal parts (60 μL each) to create final 1× concentration and incubated for 1 hour at 37° C. HepG2 cells were serum starved in serum-free EMEM for 1 hour at 37° C. Peptide: Lipid complexes (100 μL total volume per well) were transferred to HepG2 cells for 2 hours at 37° C. Cells were washed, fixed, and intracellular fluorescence was read on Promega GloMax at 475 nm excitation, 500-550 emission. Fluorescence values were background corrected from no treatment conditions. Data are presented as mean+SD for two runs. The results of FIG. 17 and FIG. 18 show that many test peptides have cholesterol import activity (corresponding to cellular fluorescence values 20 RFU above baseline) in HepG2 cells, including T-007, T-009, T-012, T-013, T-018, T-021, T-024, T-025, T-027, T-028, T-029, T-030, T-044, T-065, T-066, T-068, T-070, and T-080.

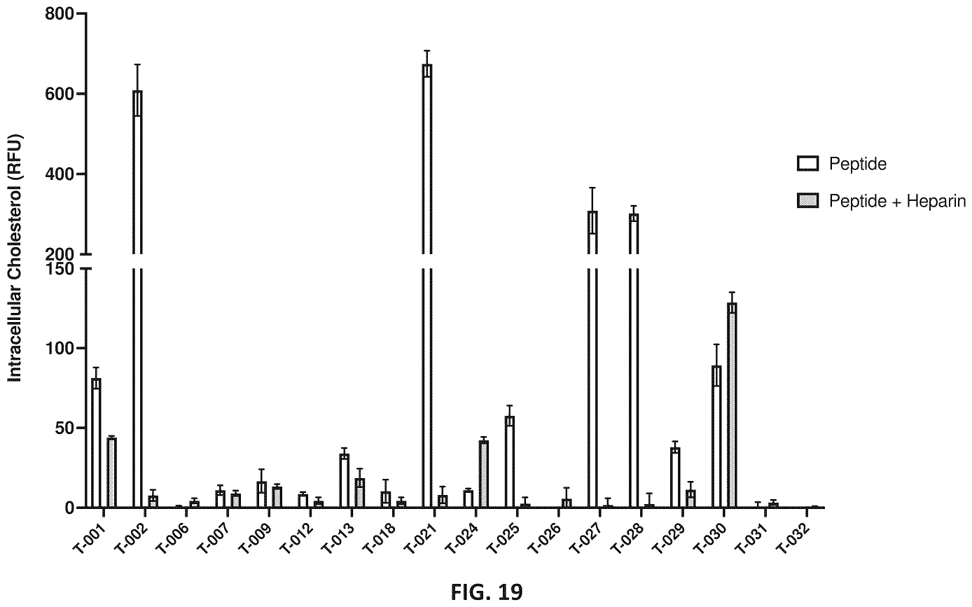

FIG. 19 shows HepG2 GAG dependent cholesterol import in a subset of the peptides. HepG2 cells were seeded on 96-well plates at 10,000 cells per well and grown to confluence. A 2× solution of 500 μM DMPC and 50 μM BODIPY-Cholesterol with or without 100 μg/mL heparin was prepared in serum free EMEM. Peptides were diluted to 20 μM in serum-free EMEM (a 2× solution). Peptides were added to lipid solution in equal parts (60 μL each) and incubated for 1 hour at 37° C. HepG2 cells were serum starved in serum-free EMEM for 1 hour at 37° C. Peptide: Lipid complexes (100 μL total volume per well) were transferred to HepG2 cells for 2 hours at 37° C. Cells were washed, fixed, and intracellular fluorescence was read on Promega GloMax at 475 nm excitation, 500-550 emission. Fluorescence values were background corrected from no treatment conditions. Data are presented ±SD for 2 runs. Based on these data, test peptides T-021, T-025, T-027, T-028, and T-029 rely on GAG binding for cholesterol import activity in HepG2 cells.

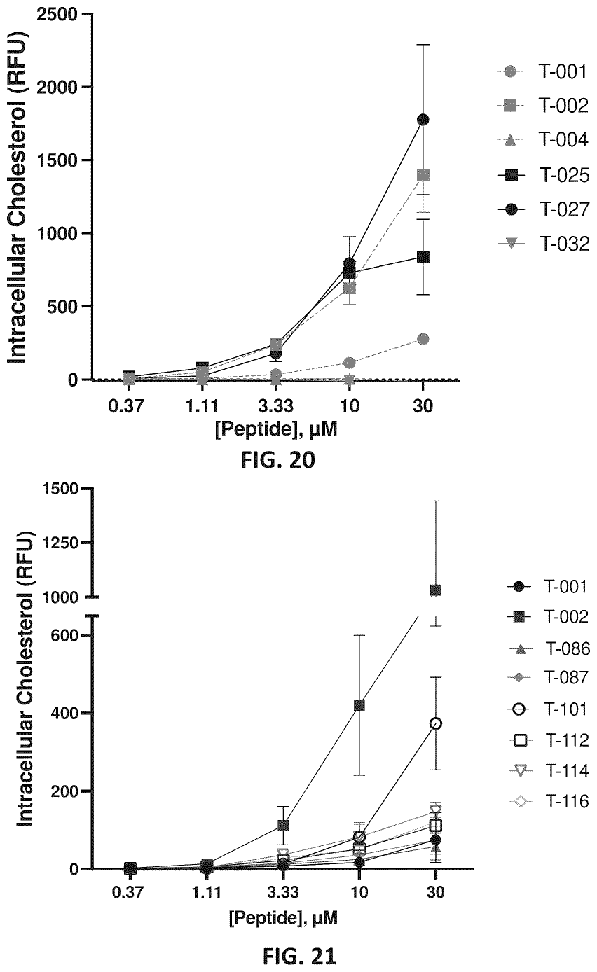

FIGS. 20 and 21 illustrate HepG2 cholesterol import titration curves for some of the peptides (T-025, T-027, T-032, T-086, T-087, T-101, T-112, T-114 and T-116), as shown. HepG2 cells were seeded on 96-well plates at 10,000 cells per well and grown to confluence. Peptide: Lipid complexes were created by preparing a solution of serum-free EMEM containing 30 μM peptide, 750 μM DMPC, and 75 μM BODIPY-Cholesterol. Peptide: Lipid complexes were serially diluted 1:3 for 5 concentrations, and incubated for 1 hour at 37° C. HepG2 cells were serum starved in serum free EMEM for 1 hour at 37° C. Peptide: Lipid complexes (100 μL total volume per well) were transferred to HepG2 cells for 2 hours at 37° C. Cells were washed, fixed, and intracellular fluorescence was read on Promega GloMax at 475 nm excitation, 500-550 emission. Fluorescence values were background corrected from no treatment conditions. Data are presented as mean+SD for two runs. Test peptides T-025, T-027, T-101, T-112, T-114, and T-116 demonstrated concentration-dependent cholesterol import activity in HepG2 cells.

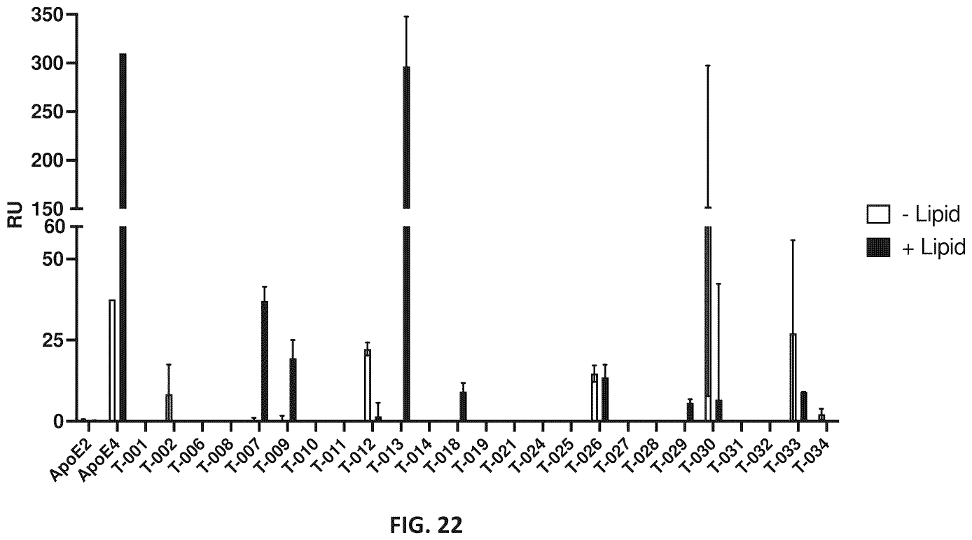

FIG. 22 shows lipid-dependent LDLR binding (SPR) for a subset of the peptides. Peptides were diluted in 1×HBS-N buffer with 1 mM CaCl2 to a final concentration of 1 μM and incubated with or without 25 μM DMPC for 1 hour at RT to form lipid complexes. Each peptide was tested in duplicate. LDLR was diluted to 10 μg/mL in 10 mM acetate buffer pH 4.5 and was immobilized (˜400RU) on a CM5 chip via amine coupling utilizing 1×HBS-P+ buffer with 1 mM CaCl2. Test peptides (with or without DMPC) were exposed to immobilized LDLR at a flow rate of 30 μL/sec for 120 seconds in HBS-N with 1 mM CaCl2. Between cycles the surface was regenerated with 3, 30 second injections of 10 mM NaOH and 100 mM EDTA. Data are plotted as max RU values, averaged from three separate runs. Test peptides T-007, T-009, T-013, T-018, and T-029 demonstrated lipid-dependent LDLR binding activity, where RU values for peptide binding to LDLR in the presence of lipid was higher than the peptide alone without lipid.

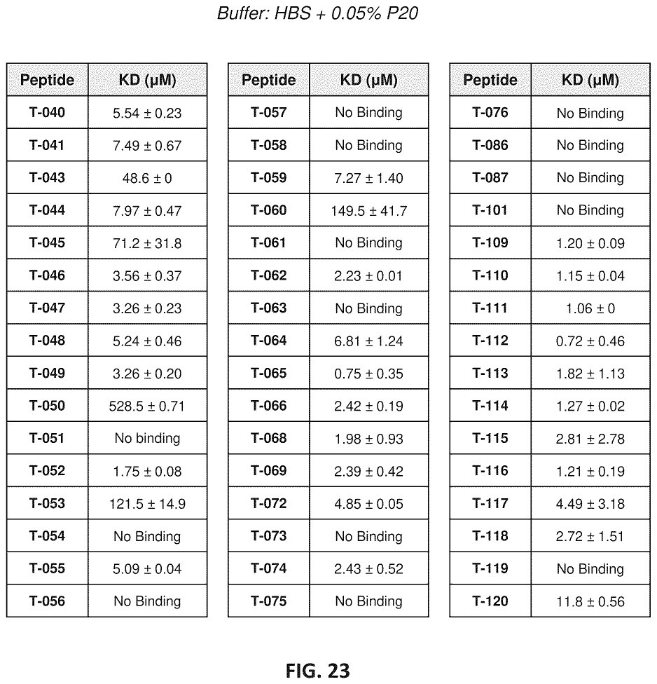

The table shown in FIG. 23 illustrates LDLR binding data (SPR), using a high surfactant preparation. Recombinant LDLR was diluted in 10 mM acetate buffer pH 4.5 and amine coupled to a CM5 chip at 1000 RU. Peptides were diluted to 5 μM in running buffer (HBS-P+, 1 mM CaCl2) and serially diluted 1:3 for 5 total concentrations. Peptides were exposed to immobilized LDLR at a rate of 30 μL/sec for 120 seconds. Between cycles, the chip surface was regenerated with 3 injections of 50 mM NaOH/100 mM EDTA. Multi-Cycle Kinetics was performed on a Biacore Insight Evaluation Software and binding kinetics were analyzed with a 1:1 binding model. Several test peptides had binding KD<10 μM to LDLR in this assay, including T-040, T-041, T-044, T-046, T-047, T-048, T-049, T-052, T-055, T-059, T-062, T-064, T-065, T-066, T-068, T-069, T-072, T-074, T-109, T-110, T-111, T-112, T-113, T-114, T-115, T-116, T-117, T-118, and T-120.

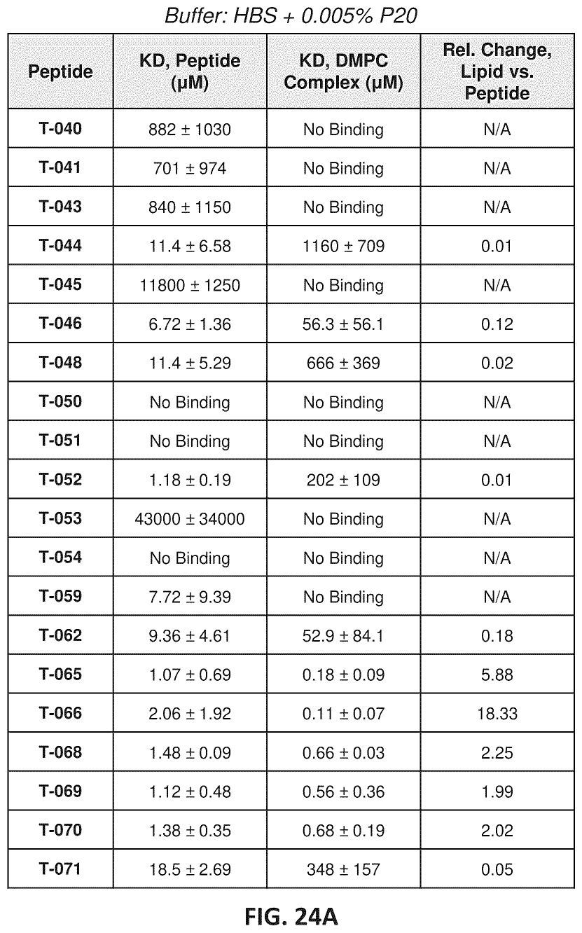

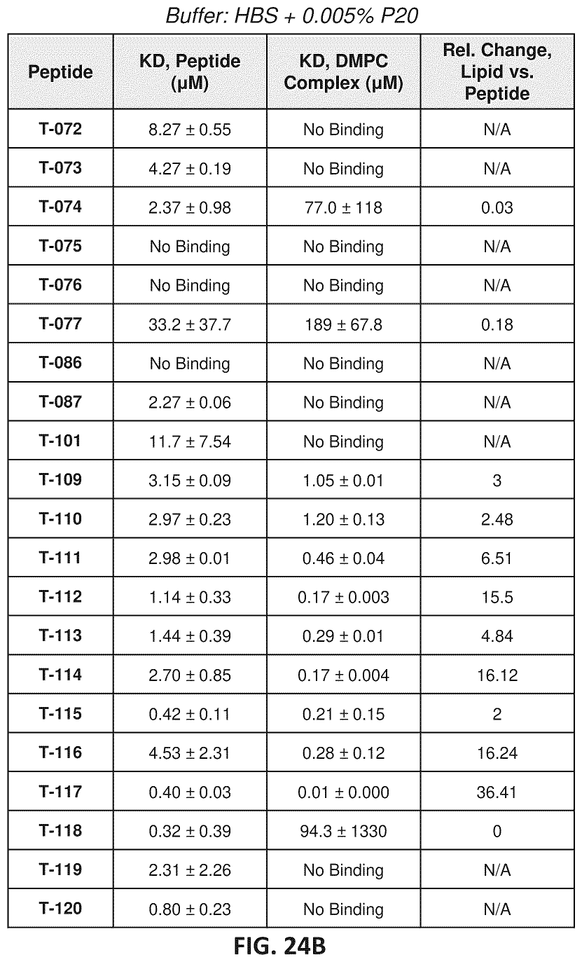

The table of FIGS. 24A-24B summarizes the results of LDLR binding data (SPR), for lipid-dependent binding. Recombinant LDLR was diluted in 10 mM acetate buffer pH 4.5 and amine coupled to a CM5 chip at 1000 RU. Peptides were diluted to 5 μM in running buffer (HBS-P, 1 mM CaCl2) and serially diluted 1:3 for 5 total concentrations. In a separate preparation, peptides were diluted to 1 μM in running buffer containing 25 μM DMPC and serially diluted 1:3 for 5 total concentrations in running buffer. Peptide: DMPC complexes were incubated for 1 hour prior to use. Peptides or peptide: lipid complexes were exposed to immobilized LDLR at a rate of 30 μL/sec for 120 seconds. Between cycles, the chip surface was regenerated with 3 injections of 50 mM NaOH/100 mM EDTA. Multi-Cycle Kinetics was performed on a Biacore Insight Evaluation Software and binding kinetics were analyzed with a 1:1 binding model. Several test peptides had a >2× change in LDLR binding in the presence of DMPC lipid, including T-065, T-066, T-068, T-070, T-109, T-110, T-111, T-112, T-113, T-114, T-115, T-116, and T-117.

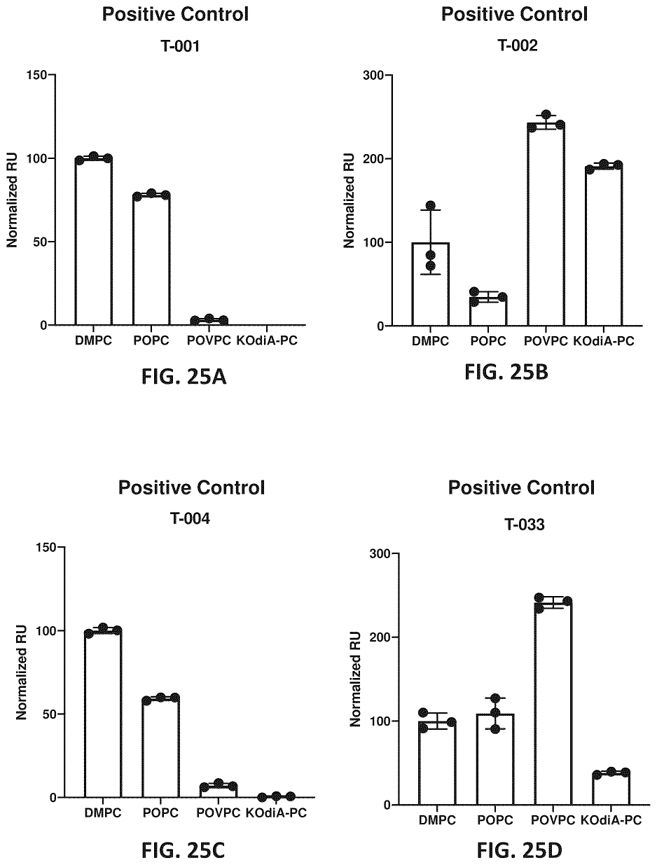

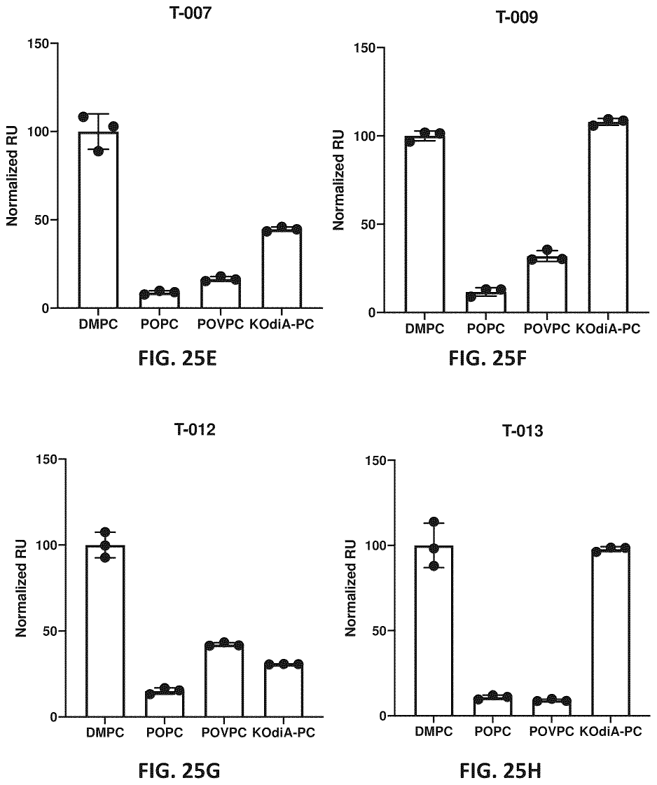

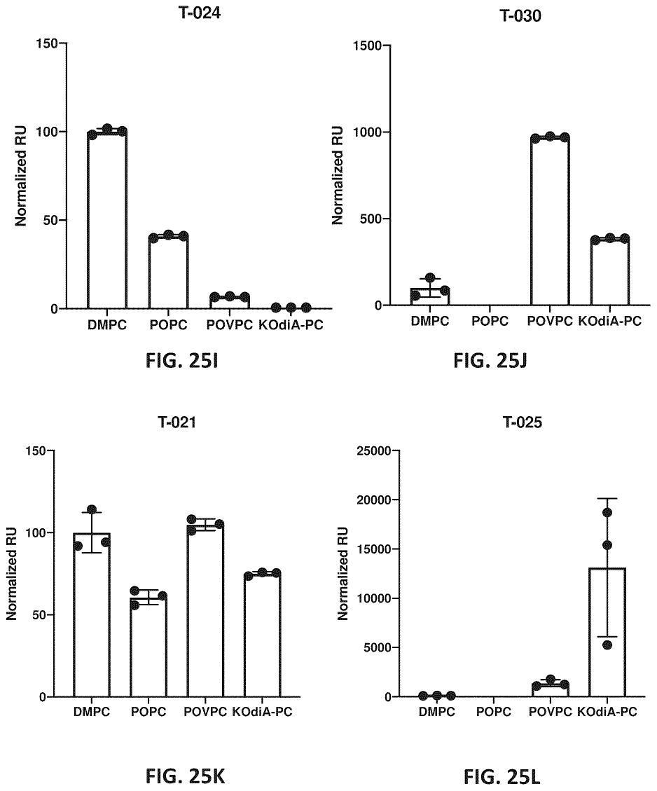

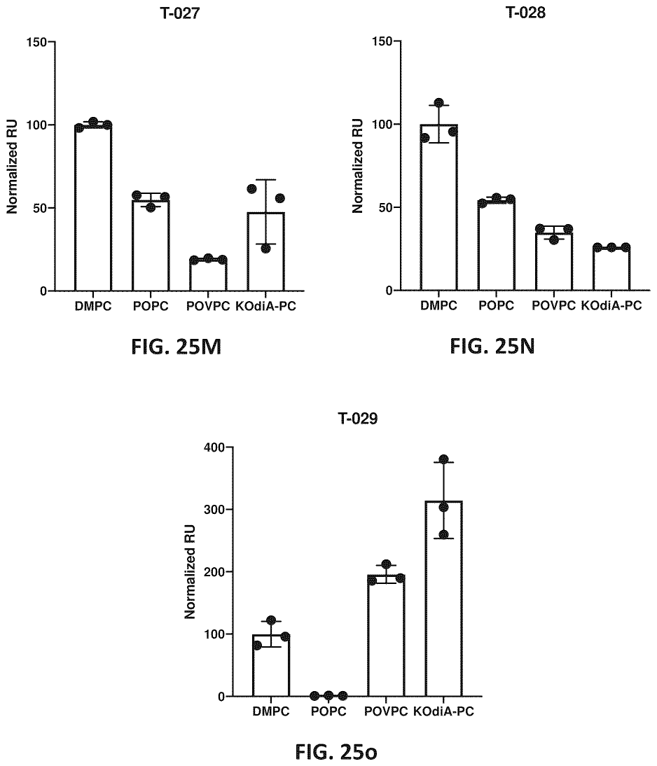

FIGS. 25A-25O show peptide binding activity to non-oxidized and oxidized lipids via SPR for indicated peptides. Peptides were reconstituted to 50 μg/mL in 10 mM acetate buffer, pH 4.5 and immobilized to ˜200 RU on a CM5 chip via amine coupling. Non-oxidized lipids (DMPC & POPC) and oxidized lipids (POVPC & KOdiA-PC) were diluted to 100 M in HBS-EP running buffer and exposed to the chip for binding (n=3) at a flow rate of 30 μL/sec for 120 seconds. The surface was regenerated with 20% EtOH. T-001 served as a positive control and T-031 served as a negative control on each chip. For each peptide, the RU response for all lipids was normalized to the DMPC RU values. If the peptide was unable to bind DMPC, it was deemed inactive from immobilization. Test peptides T-009, T-013, T-021, T-025, T-029, and T-030 all demonstrated binding to at least one oxidized lipid that was equivalent or improved to binding against DMPC.

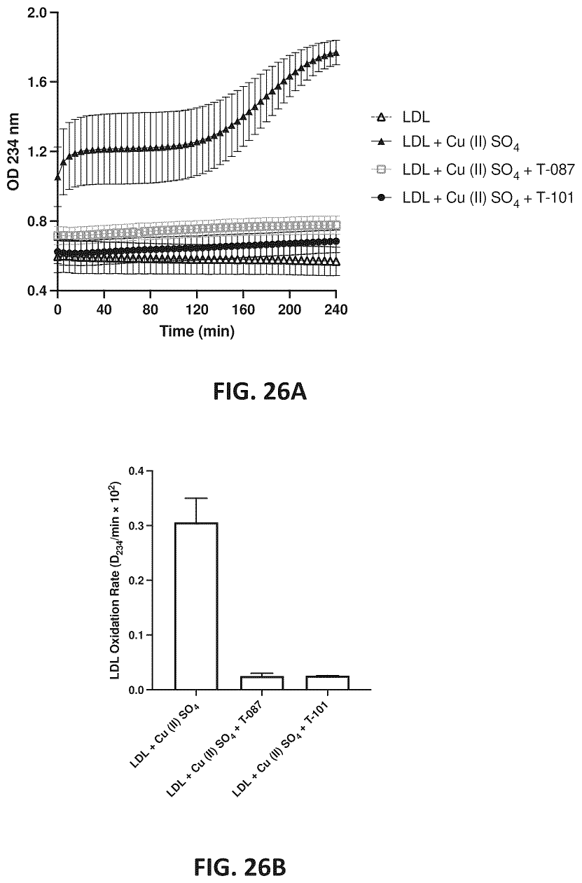

FIGS. 26A-26B show the results of oxidized LDL testing. In 96-well microplates, LDL (stock concentration 10 mg/mL), copper (II) sulfate (stock concentration 1.5 mM), and peptides (stock concentration 1 mM) were combined in sextuplicate wells to final concentrations of 100 μg/mL, 15 μM, and 25 μM, respectively. Control wells were filled with LDL (100 μg/mL) and copper (II) sulfate (15 μM), or LDL alone. The microplate was immediately read in a plate reader at OD 234 nm every 5 min for 240 minutes. An increase in absorbance at 234 nm reflects an increase in conjugated diene formation and is used as a measure of oxidation. In FIG. 26A data are presented as mean+SD over three technical replicates. In FIG. 26B, LDL oxidation rate was calculated as maximum absorbance divided by the length of the lag period, and data are presented as mean+SD over three technical replicates. Test peptides T-087 and T-101 decreased the oxidation of LDL in the presence of copper (II) sulfate in this assay by 92% and 91.6%, respectively.

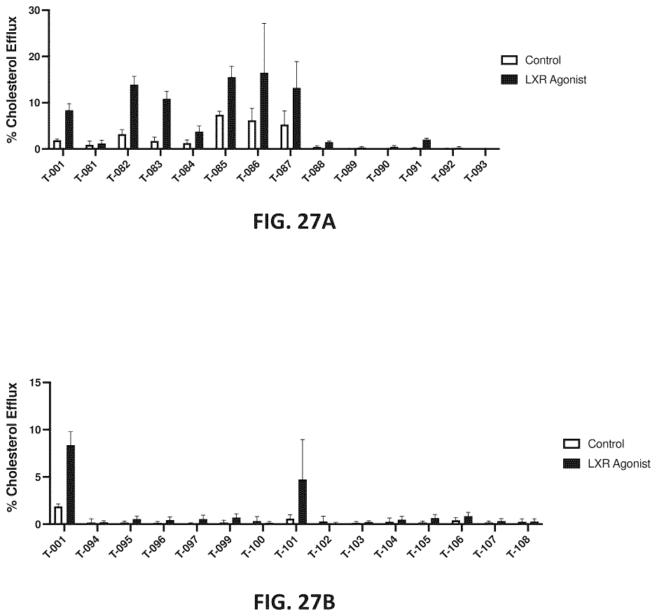

FIGS. 27A-27B and FIG. 28 illustrate HMC3 cholesterol efflux. Fully confluent 96-well plates of HMC3 cells were treated for 1 hour in serum-free EMEM containing 10 mM methyl-β-cyclodextrin, 100 μM cholesterol, and 25 μM BODIPY cholesterol. Cells were then washed and incubated overnight in serum free EMEM containing 1 μM LXR agonist GW3965, 2 μg/mL ACAT inhibitor, and 0.2% BSA (LXR Agonist treated), or serum-free EMEM containing 2 μg/mL ACAT inhibitor, and 0.2% BSA (Control). Cells were washed and incubated with 20 μM peptides diluted in serum-free Fluorobrite DMEM for 4 hours at 37° C. Media was transferred to a new 96-well plate and fresh serum-free Fluorobrite DMEM was placed on the cells. Media and cellular fluorescence were read on a Promega GloMax at 475 nm excitation, 500-550 emission. Fluorescence values were background corrected from no treatment conditions and percent efflux was calculated by the following equation: % efflux=(Media Fluorescence values)/(Media+Cell Fluorescence values). Data are presented as mean+SD over two runs per peptide. Several test peptides induced greater cholesterol efflux in the presence of LXR agonist in HMC3 cells, including T-084, T-085, T-086, T-087, T-101, as well as T-101 derivative peptides T-121, T-122, T-123, T-124, T-126, T-128, T-129, T-134, T-135, T-136, T-137, T-138, T-139, T-140, T-141, T-142, T-143, T-144, T-145, T-146, T-148, T-149, T-150 and T-151.

FIG. 29 shows ARPE-19 cholesterol efflux for the indicated peptides. Fully confluent 96-well plates of ARPE-19 cells were treated for 1 hour in serum-free DMEM containing 10 mM methyl-β-cyclodextrin, 100 μM cholesterol, and 25 μM BODIPY cholesterol. Cells were then washed and incubated overnight in serum-free DMEM containing 1 μM GW3965, 2 μg/mL ACAT inhibitor, and 0.2% BSA (LXR Agonist treated), or serum free DMEM containing 2 μg/mL ACAT inhibitor, and 0.2% BSA (Control). Cells were washed and incubated with 20 μM peptides diluted in serum-free Fluorobrite DMEM for 4 hours at 37° C. Media was transferred to a new 96-well plate and fresh serum-free Fluorobrite DMEM was placed on the cells. Media and cellular fluorescence were read on a Promega GloMax at 475 nm excitation, 500-550 emission. Fluorescence values were background corrected from no treatment conditions and percent efflux was calculated by the following equation: % efflux=(Media Fluorescence values)/(Media+Cell Fluorescence values). Data are presented as mean+SD over two runs per peptide. Several T-087 derivative peptides demonstrated increased cholesterol efflux in ARPE-19 cells following LXR agonist treatment, including T-152, T-153, T-154, T-155, T-156, T-157, T-158, T-159, T-160, T-161, T-162, T-163, T-164, T-166, T-167, T-168, T-170, T-171, T-172, T-174, T-175, T-176 and T-178.

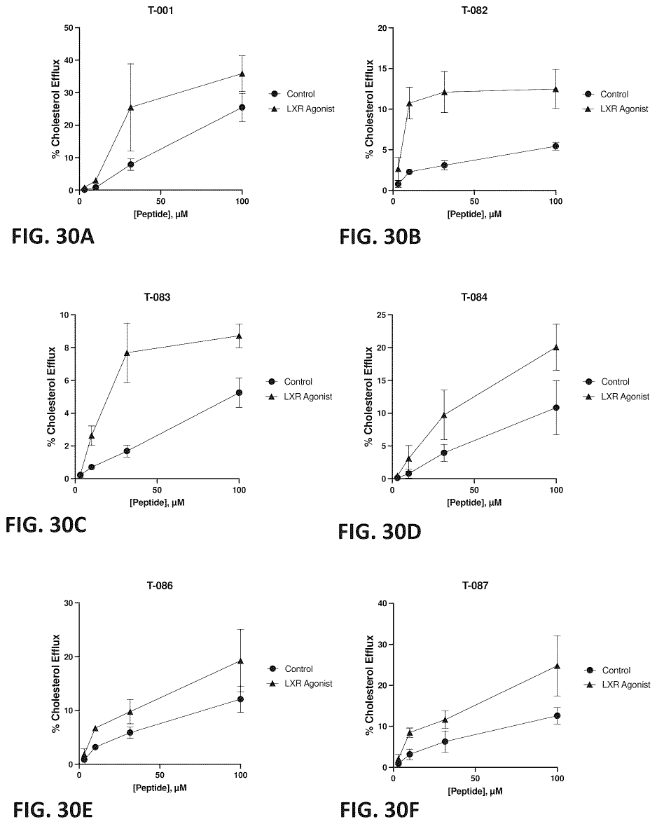

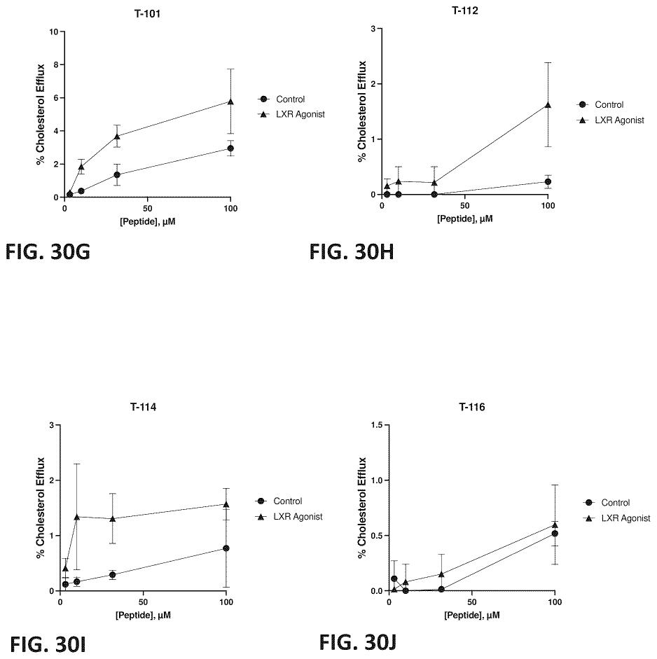

FIGS. 30A-30J show HMC3 cholesterol efflux titration curves for the indicated peptides. Fully confluent 96-well plates of HMC3 cells were treated for 1 hour in serum-free EMEM containing 10 mM methyl-β-cyclodextrin, 100 μM cholesterol, and 25 μM BODIPY cholesterol. Cells were then washed and incubated overnight in serum free EMEM containing 1 μM GW3965, 2 μg/mL ACAT inhibitor, and 0.2% BSA (LXR Agonist treated), or serum free EMEM containing 2 μg/mL ACAT inhibitor, and 0.2% BSA (Control). Cells were washed and incubated with 0-100 μM peptides serially diluted in serum-free Fluorobrite DMEM for 4 hours at 37° C. Media was transferred to a new 96-well plate and fresh serum-free Fluorobrite DMEM was placed on the cells. Media and cellular fluorescence were read on a Promega GloMax at 475 nm excitation, 500-550 emission. Fluorescence values were background corrected from no treatment conditions and percent efflux was calculated by the following equation: % efflux=(Media Fluorescence values)/(Media+Cell Fluorescence values). Data are presented as mean+SD over two runs per peptide. Peptides tested were (A) T-001, positive control; (B) T-082, positive control; (C) T-083, positive control; (D) T-084; (E) T-086; (F) T-087; (G) T-101; (H) T-112; (I) T-114; and (J) T-116. All test peptides demonstrated a concentration-dependent increase in cholesterol efflux in HMC3 cells in the presence of LXR agonist.

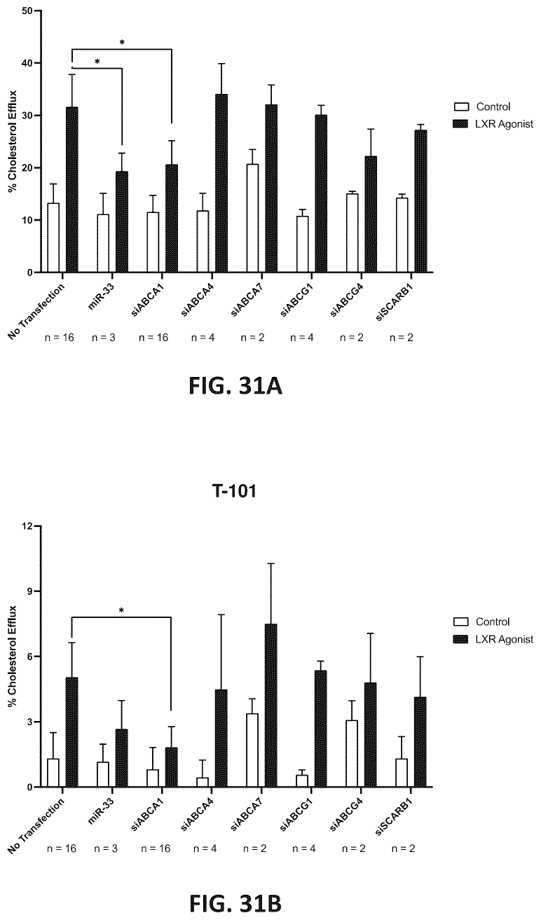

FIGS. 31A-31B show cholesterol efflux following ATP-binding cassette family knockdown assays. ARPE-19 cells were seeded at 25,000 cells per well in 96-well plates or 100,000 cells per well in 12-well plates and allowed to grow to confluence for 1 week. miR-33a (which targets ABCA1) was diluted to 30 nM in Opti-MEM. siRNAs for ABCA1, ABCA4, ABCA7, ABCG1, ABCG4, or SRB1 were diluted in Opti-MEM to a final concentration of 100 nM siRNA. RNAiMax Lipofectamine was added to the siRNA or miRNA preparations for 5 min prior to transfection for 24 hours at 37° C. in serum-free DMEM. To validate siRNAs or miRNAs used in this experiment, qPCR was performed to verify at least 85% knockdown of the target transcript, and verify there was no effect on other transcripts tested. Following siRNA or miRNA treatment, cells were treated for 1 hour in serum-free DMEM containing 10 mM methyl-β-cyclodextrin, 100 μM cholesterol, and 25 μM BODIPY cholesterol. Cells were then washed and incubated overnight in serum-free DMEM containing 1 μM LXR agonist GW3965, 2 μg/mL ACAT inhibitor, and 0.2% BSA (LXR Agonist treated), or serum free EMEM containing 2 μg/mL ACAT inhibitor, and 0.2% BSA (Control). Cells were washed and incubated with 20 μM of peptides T-087 or T-101 diluted in serum-free Fluorobrite DMEM for 4 hours at 37° C. Media was transferred to a new 96-well plate and fresh serum-free Fluorobrite DMEM was placed on the cells. Media and cellular fluorescence were read on a Promega GloMax at 475 nm excitation, 500-550 emission. Fluorescence values were background corrected from no treatment conditions and percent efflux was calculated by the following equation: % efflux=(Media Fluorescence values)/(Media+Cell Fluorescence values). Data are presented as mean+SD from two runs (siABCA7, siABCG4, siSCARB1), three runs (miR-33), four runs (siABCA4, siABCG1), or 16 runs (siABCA1). Differences were tested with a Two-Way ANOVA with Tukey's post hoc testing. *, Significantly Different (P<0.05). Peptides T-087 and T-101 demonstrated reduced cholesterol efflux following the knockdown of miR-33 and siABCA1, but not any other tested siRNA, indicating these peptides primarily direct cholesterol efflux activity through ABCA1.

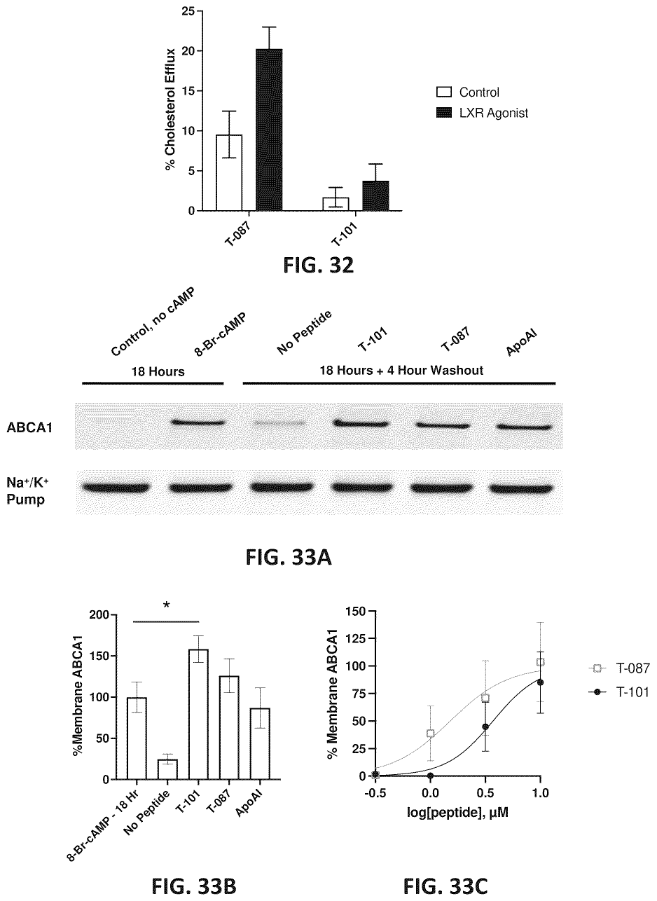

FIG. 32 is a graph illustrating the cholesterol efflux in iPS-RPE Cells for two examples, T-087 and T-101. iPS-derived RPE cells were obtained from Fujifilm (iCell Retinal Pigment Epithelial Cells) and grown for 28 days on 96-well dishes using vitronectin as a coating substrate. Cells were verified for cobblestone morphology and pigmentation, then cells were treated for 1 hour in serum-free DMEM containing 10 mM methyl-β-cyclodextrin, 100 μM cholesterol, and 25 μM BODIPY cholesterol. Cells were then washed and incubated overnight in serum free DMEM containing 1 μM LXR agonist GW3965, 2 g/mL ACAT inhibitor, and 0.2% BSA (LXR Agonist treated), or serum free EMEM containing 2 μg/mL ACAT inhibitor, and 0.2% BSA (Control). Cells were washed and incubated with 20 μM of peptides serially diluted in serum-free Fluorobrite DMEM for 4 hours at 37° C. Media was transferred to a new 96-well plate and fresh serum-free Fluorobrite DMEM was placed on the cells. Media and cellular fluorescence were read on a Promega GloMax at 475 nm excitation, 500-550 emission. Fluorescence values were background corrected from no treatment conditions and percent efflux was calculated by the following equation: % efflux=(Media Fluorescence values)/(Media+Cell Fluorescence values). Data are presented as mean+SD from two runs. Peptides T-087 and T-101 were able to increase cholesterol efflux in iPS-RPE cells following LXR agonism.

FIGS. 33A-33C illustrate the results of membrane ABCA1 stability testing in J774 Cells. J774 murine macrophage cells were seeded at 50,000 cells per well on 12-well plates and were allowed to grow to confluence. A vehicle solution of 0.1% BSA in serum-free DMEM was prepared for control wells. An ABCA1 induction solution of 0.1% BSA with 1 mM 8-Br-CAMP and 50 μg/mL AcLDL was prepared in serum-free DMEM and incubated on cells overnight at 37° C. to increase ABCA1 protein levels on the membrane. A solution of 10 μM peptide was prepared in serum-free DMEM and serially diluted 1:10 for 4 concentrations. ApoAI was prepared at a concentration of 300 nM. Peptides or ApoA1 were added to cells for 4 hours at 37° C. before isolating membrane protein and measuring membrane ABCA1 levels by western blot. Blots were quantified in ImageJ and the signal was normalized to membrane Na+/K+ pump levels. Signals were further normalized to the 18-hour cAMP treated group to determine percent (%) Membrane ABCA1 remaining. In the absence of peptide or ApoA1 treatment, membrane ABCA1 levels were reduced following 4 hour washout of 8-Br-CAMP, whereas treatment with either peptides T-087, T-101, or ApoA1 preserved membrane ABCA1 levels. The EC50 values for peptides T-087 and T-101 in this assay were 1.51 μM and 3.7 μM, respectively.

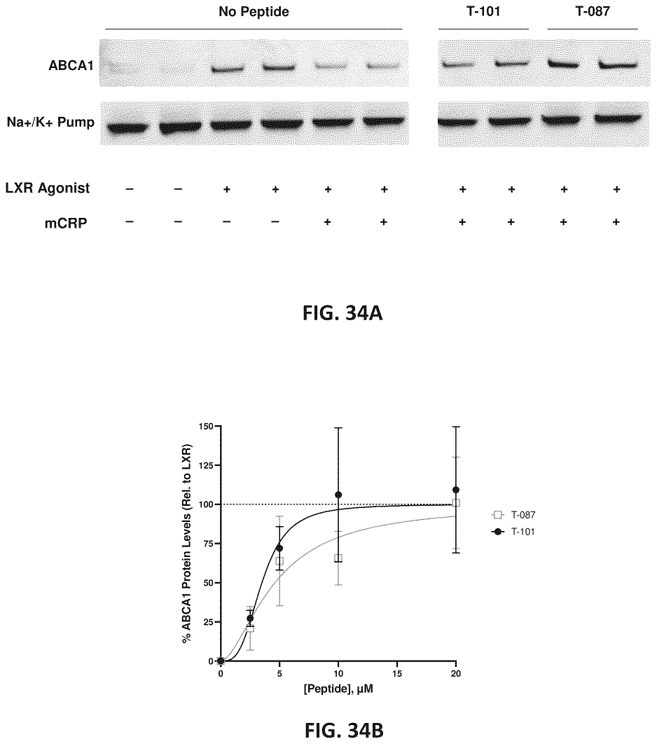

FIG. 34 shows membrane ABCA1 stability testing results in ARPE-19 cells for peptides T-087 and T-101. ARPE-19 cells were seeded at 100,000 cells per well on 12-well plates and were allowed to grow to confluence. To prepare monomeric CRP (mCRP), recombinant CRP was prepared in a PBS solution containing 8 M Urea and 10 mM EDTA and incubated at 37° C. for 2 hours prior to desalting with a Zeba spin column (7K MW cutoff). mCRP was prepared to a 2× solution of 20 μg/mL. A 2× solution of 40 μM peptides was prepared in serum-free DMEM and serially diluted 1:2 over 4 concentrations. mCRP and peptides were added 1:1 to cells for 24 hours at 37° C. before isolating membrane protein and measuring membrane ABCA1 levels by western blot. Blots were quantified in ImageJ. Signals were further normalized to the LXR agonist treated group to determine percent (%) ABCA1 remaining. Treatment with LXR agonist increased membrane ABCA1 levels in this assay, but following treatment with mCRP, membrane ABCA1 levels were decreased. In the presence of peptides T-087 or T-101 during the mCRP incubation period, ABCA1 membrane levels were preserved compared to the no peptide treatment. The EC50 values for peptides T-087 and T-101 in this assay were 4.6 μM and 3.5 μM, respectively.

FIGS. 35 and 36 show membrane ABCA1 stability testing using ARPE-19 cells. ARPE-19 cells were seeded at 100,000 cells per well on 12-well plates and were allowed to grow to confluence. To prepare monomeric CRP (mCRP), recombinant CRP was prepared in a PBS solution containing 8 M Urea and 10 mM EDTA and incubated at 37° C. for 2 hours prior to desalting with a Zeba spin column (7K MW cutoff). mCRP was prepared to a 2× solution of 20 μg/mL. A 2× solution of 40 μM peptides was prepared in serum-free DMEM. CRP and peptides were added 1:1 to cells for 24 hours at 37° C. before isolating membrane protein and measuring membrane ABCA1 levels by western blot. Blots were quantified in ImageJ. In FIG. 35, Signals were further normalized to the T-087 treated group to determine percent (%) ABCA1 levels compared to peptide T-087 treated cells. In FIG. 36, Signals were further normalized to the peptide T-101 treated group to determine percent (%) ABCA1 levels compared to peptide T-101 treated cells. Several derivatives of peptide T-087 resulted in reduction of membrane ABCA1 levels following mCRP treatment compared to peptide T-087 treated cells, including peptides T-155, T-156, T-157, T-158, T-159, T-162, T-166, T-167, T-170, T-173, and T-177, indicating residues 3, 4, 5, 6, 7, 9, 13, 14, 15 in peptide T-087 contribute to activity of stabilizing membrane ABCA1 following mCRP treatment. Several derivatives of peptide T-101 also resulted in reduction of membrane ABCA1 levels following mCRP treatment compared to peptide T-101 treated cells, including peptides T-121, T-124, T-126, T-127, T-128, T-130, T-132, T-134, T-135, T-137, T-138, T-104, T-141, T-145, T-148, T-149, and T-150, indicating that residues at positions 1, 4, 6, 7, 8, 10, 12, 14, 15, 17, 18, 21, as well as groups of residues between positions 6-19 are critical in T-101 for full activity of stabilizing ABCA1 following mCRP treatment.

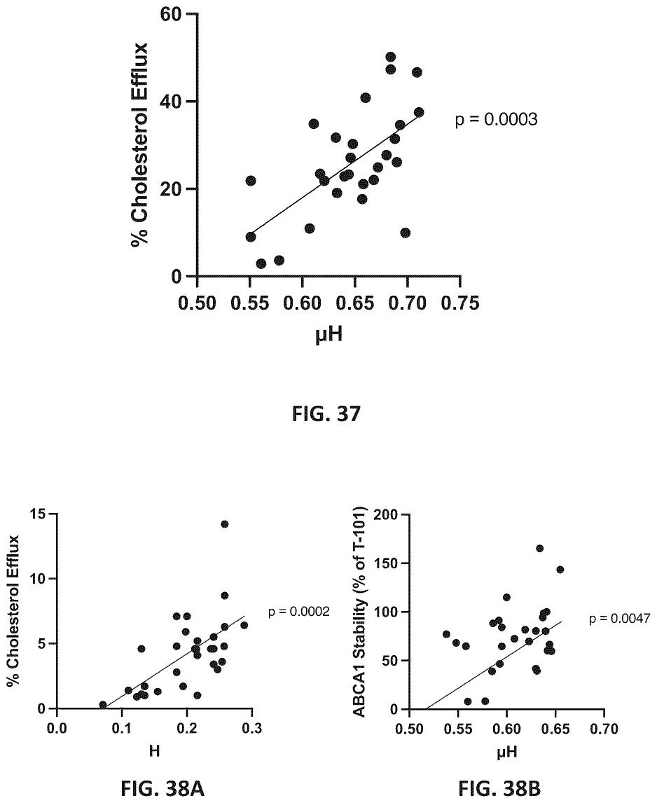

FIG. 37 shows the correlation of cholesterol efflux and hydrophobic moment for peptides T-152-T-178 (SEQ IDs 152-178). In this example a linear regression was used to determine slope deviation from zero. The source data in this figure comes from FIG. 28, as well as the calculated hydrophobic moment, defined in Eisenberg (Eisenberg et al. 1982. The helical hydrophobic moment: a measure of the amphiphilicity of a helix. Nature). The results of this analysis show that the hydrophobic moment of test peptides is positively correlated with their cholesterol efflux activity.

FIG. 38A shows the correlation of cholesterol efflux and hydrophobicity for peptides T-121 to T-151 (SEQ IDs 121-151). FIG. 38B shows the correlation of ABCA1 membrane stability and hydrophobic moment for these peptides. Linear regression was used to determine slope deviation from zero. Source data for these figures comes from FIG. 29 and FIG. 36. Hydrophobicity is defined in Fauchere and Pliska. (Fauchere and Plisk (1983). Hydrophobic Parameters II of Amino-Acid Side Chains from the Partitioning of N-Acetyl-Amino-Acid Amides. Eur J Med Chem). Hydrophobic moment is defined in Eisenberg (Eisenberg et al. 1982. The helical hydrophobic moment: a measure of the amphiphilicity of a helix. Nature). The results of the analysis shown in FIG. 38A show that the hydrophobicity of test peptides is positively correlated with their cholesterol efflux activity. The results of the analysis shown in FIG. 38B indicate that the membrane ABCA1 stability is positively associated with their hydrophobic moment.

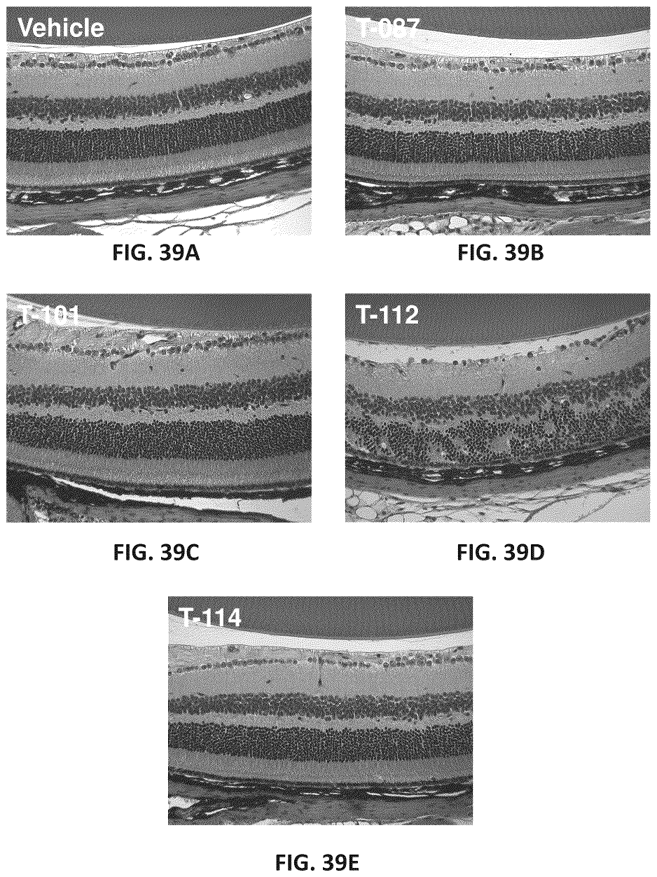

FIGS. 39A-39E shows examples of the results of tolerability testing of representative peptides in mice. T-087, T-101, T-112, and T-114 were prepared in sterile H2O+0.001% Tween-80 to a concentration of 520 μM for intravitreal (IVT) delivery into 12-week-old C57BL/6J mice (n=3 mice per group). Seven days following IVT injection, eyes were harvested and prepared for hematoxylin and eosin staining. A blinded veterinarian performed histopathological analysis of tissue slices to determine whether the peptides induced inflammation or retinal disruption. Representative images of each peptide are shown, indicating all peptides except T-112 were well tolerated.

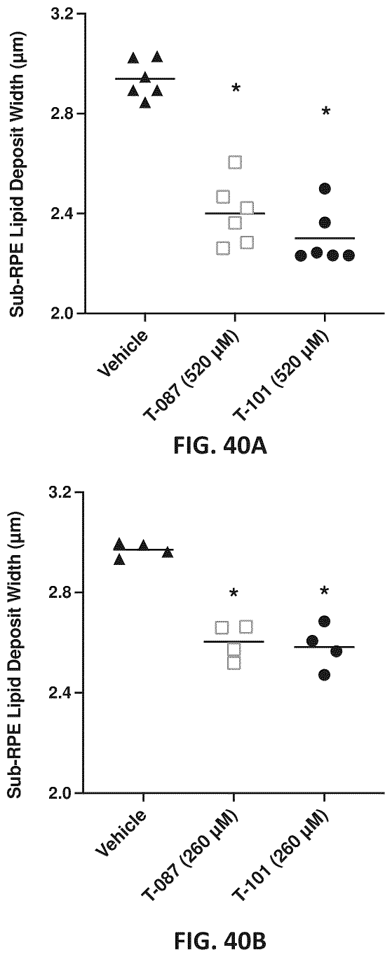

FIGS. 40A and 40B show results from in vivo studies measuring the effect of peptides T-087 and T-101 in reducing sub-RPE BODIPY+ lipid deposition in ApoE^{-/-} mice. FIG. 40A shows the results from a study where three-month-old ApoE−/− mice (B6.129P2-Apoe^{tmlUnc}/J) were given a high cholesterol diet (Research Diets D12079B) for two months. Two weeks prior to sacrifice, mice were randomized (n=6 mice per group) and received 1 μL IVT injections of 520 μM peptide T-087 or peptide T-101, or vehicle control. At sacrifice, eyes were enucleated and stained with BODIPY to mark neutral lipids, then imaged at 20× magnification. ImageJ was used to quantify the area of sub-RPE BODIPY+ staining, then the area of interest (AOI) was divided by the length of the region, yielding an average width of lipid deposition. Nine images were averaged for each mouse. Data are presented as individual data points representing values for individual mice. Differences were tested with a One-Way ANOVA with Tukey's post hoc testing. *, Different from Vehicle (P<0.05). In a separate study, the results of which are summarized in FIG. 40B, three-month-old ApoE−/− mice (B6.129P2-Apoe^{tmlUnc}/J) were given a high cholesterol diet (Research Diets D12079B) for two months. Two weeks prior to sacrifice, mice were randomized (n=4 mice per group) and received 1 μL IVT injections of 260 μM peptide T-087 or peptide T-101, or vehicle control. At sacrifice, eyes were enucleated and stained with BODIPY to mark neutral lipids, then imaged and quantified as above. Twelve images were averaged for each mouse. Data are presented as individual data points representing values for individual mice. Differences were tested with a One-Way ANOVA with Tukey's post hoc testing. *, Different from Vehicle (P<0.05). In both FIG. 40A and FIG. 40B, treatment with test peptide T-087 or test peptide T-101 resulted in statistically significant decrease of sub-RPE lipid deposits compared to vehicle-treated mice.

### Therapeutic Peptide Examples

The therapeutic peptides described herein may therefore solubilize lipids, may induce lipid efflux from cells and may accept and transport lipids that are released from within a cell. These therapeutic peptides may be engineered mimetics of one or more apolipoproteins such as ApoA1 with a structural homology to endogenous ApoA1 such that the structure and function of the therapeutic peptide is amphipathic in nature and effectively enhances the activities of cholesterol efflux regulatory proteins or transporter proteins involved in lipid efflux or lipid processing mechanisms (e.g., ABCA1, ABCG1, SR-B1 or related proteins). The therapeutic peptides described herein may interact with one or more molecules involved in lipid transport and processing pathways such as lipoproteins, lipid efflux transporters and lipoprotein particle modifiers. For example, where the therapeutic peptide provides for a mimetic structurally similar to ApoA1, the therapeutic peptide may associate with and support HDL function as it relates to lipid processing mechanisms and cholesterol efflux pathways. Many of the therapeutic peptides listed in FIGS. 1B-1I demonstrate these properties. In general, the therapeutic peptides may be 80 amino acids or less (e.g., 75 or fewer amino acids, 70 or fewer amino acids, 65 or fewer amino acids, 60 or fewer amino acids, 55 or fewer amino acids, 50 or fewer amino acids, 49 or fewer amino acids, 48 or fewer amino acids, 47 or fewer amino acids, 46 or fewer amino acids, 45 or fewer amino acids, 44 or fewer amino acids, 43 or fewer amino acids, 42 or fewer amino acids, 41 or fewer amino acids, 40 or fewer amino acids, 35 or fewer amino acids, 30 or fewer amino acids, 25 or fewer amino acids, 22 or fewer amino acids, 18 or fewer amnio acids, etc.).

The therapeutic peptides described herein may therefore solubilize lipids and may transport the captured lipids into cells. The therapeutic peptides may utilize GAG-dependent, GAG-independent and/or lipid import receptors (e.g., LDLR, SR-B1, and related receptors) for facilitating lipid import into cells. The therapeutic peptides may have the property of binding to oxidized lipids, which may enable clearance of pro-inflammatory and/or toxic oxidized lipid species. The therapeutic peptides may have the property of binding to LDLR in a lipid-dependent manner, to ensure peptides do not bind to LDLR in the absence of lipid, which may inhibit LDLR function and disrupt cholesterol homeostasis. Many of the peptides listed in FIGS. 1B-I and shown in sequence listing as SEQ ID NOS. 5-30, 35-80, and 84-178 demonstrate these properties. Some of the therapeutic peptides include a terminal peptide region that is homologous to (in some cases identical to, in some examples identical but for or fewer mismatches out of 11 amino acids) to the peptide sequence shown in SEQ ID NO. 8 (e.g., HLRKLRKRLLR). A second region may be linked to the terminal region. Alternatively, the D-conformation of SEQ ID NO. 8 may be positioned at the C-terminal end of the peptide. The mid-variable region shown is similar to that of SEQ ID NO 16 (which is shown appended to the terminal region as peptide SEQ ID NO. 27). A therapeutic peptide may include this sequence plus an extension peptide region of up to an additional 16 amino acids or fewer. The therapeutic peptides may have the property of binding and activating ABCA1, and also sustain the presence of ABCA1 on the membrane of cells in a pro-inflammatory environment. Many of the peptides listed in FIGS. 1B-I and shown in sequence listing as SEQ ID NOS. 5-30, 35-80, and 84-178 demonstrate these properties. Therapeutic peptides may also have N- and C-terminal modifications including acetylation and amidation.

In some examples, similar peptides may vary five or fewer of the 18 amino acids of the mid-variable region. For example, up to five of these amino acids may be varied. Substitutions or deletions may be made. In some examples, these substitutions may be conservative substitutions. In some examples the same terminal region is included coupled to a second mid-variable region. The mid-variable region may be similar to the sequence of SEQ ID NO. 20. The peptide including the terminal region and the mid-variable region is shown in SEQ ID NO. 29.

### Compositions

Any of the therapeutic peptides described herein may be used as part of a pharmaceutical composition. The pharmaceutical compositions comprising therapeutic peptides described herein may include on or more pharmaceutically acceptable carriers. The pharmaceutical compositions may be suitable for any mode of administration, for example, by intravitreal administration.

In some examples, the composition comprises a polypeptide of SEQ ID No. 5-30, 35-80, or 84-178. For example, in some examples the pharmaceutical composition includes a peptide having the sequence of SEQ ID No. 27. In some examples the pharmaceutical composition includes a peptide having the sequence of SEQ ID No. 25. In some examples the pharmaceutical composition includes a peptide having the sequence of SEQ ID No. 29 (or a modified form thereof). In some examples the pharmaceutical composition includes a peptide having the sequence of SEQ ID No. 87 (or a modified form thereof). In some examples the pharmaceutical composition includes a peptide having the sequence of SEQ ID No. 101 (or a modified form thereof). In some examples the pharmaceutical composition includes a peptide having the sequence of SEQ ID No. 8 at the N-terminal end (or at the C-terminal end in the D configuration) and a total length of less than 80 amino acids.

In some examples, the composition comprises a polypeptide of SEQ ID No. 35-80, or 84-120. For example, in some examples the pharmaceutical composition includes a peptide having the sequence of SEQ ID No. 101 (or a modified form thereof). In some examples the pharmaceutical composition includes a peptide having the sequence of SEQ ID No. 87 (or a modified form thereof). In some examples the pharmaceutical composition includes a peptide having the sequence of SEQ ID No. 100 (or a modified form thereof). In some examples the pharmaceutical composition includes a peptide having the sequence of SEQ ID No. 8 at the N-terminal end (or at the C-terminal end in the D configuration) and a total length of less than 80 amino acids

In some examples, the pharmaceutical compositions comprising a peptide as described herein (including, but not limited to those of SEQ ID No. 5-30, 35-80, or 84-178) and a pharmaceutically acceptable carrier is suitable for administration to a human subject. Such carriers are well known in the art (see, e.g., Remington's Pharmaceutical Sciences, 15th Edition, pp. 1035-1038 and 1570-1580). In some examples, the pharmaceutical composition is suitable for intravitreal injection. In some examples, the pharmaceutical composition is suitable for subretinal delivery. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multi-dosage forms. The compositions are generally formulated as sterile and substantially isotonic solutions.

In one example, the therapeutic peptides described herein are formulated into a pharmaceutical composition intended for subretinal or intravitreal injection. Such formulation involves the use of a pharmaceutically and/or physiologically acceptable vehicle or carrier, particularly one suitable for administration to the eye, e.g., by subretinal injection, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. In one example, the carrier is an isotonic sodium chloride solution. In another example, the carrier is a balanced salt solution. In one example, the pharmaceutically acceptable carrier includes surfactants, e.g., Tween-80, Tween-20, or perfluorooctane (Perfluoron liquid). If the peptide solution is to be stored long-term, it may be frozen in the presence of glycerol or Tween-20.

In certain examples of the methods described herein, the pharmaceutical composition described above is administered to the subject by subretinal injection. In other examples, the pharmaceutical composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parenteral routes of administration. Routes of administration may be combined, if desired. In certain examples, the pharmaceutical compositions of the disclosure are administered after administration of an initial loading dose of the complement system protein.

In some examples, the route of administration is chosen such that it reduces the risk of retinal detachment in the patient (e.g., intravitreal rather than subretinal administration). In some examples, intravitreal administration is chosen if the vector/composition is to be administered to an elderly adult (e.g., at least 60 years of age). In particular examples, any of the vectors/pharmaceutical compositions disclosed herein are administered to a subject intravitreally. Procedures for intravitreal injection are known in the art (see, e.g., Peyman, G. A., et al. (2009) Retina 29(7): 875-912 and Fagan, X. J. and Al-Qureshi, S. (2013) Clin. Experiment. Ophthalmol. 41(5): 500-7). Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as Povidone-Iodine (BETADINE®). A similar solution may also be used to clean the eyelid, eyelashes, and any other nearby tissues {e.g., skin). Any suitable anesthetic may be used, such as lidocaine or proparacaine, at any suitable concentration. Anesthetic may be administered by any method known in the art, including without limitation topical drops, gels or jellies, and subconjuctival application of anesthetic. Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site. During injection, the needle may be inserted perpendicular to the sclera and pointed to the center of the eye. The needle may be inserted such that the tip ends in the vitreous, rather than the subretinal space. Any suitable volume known in the art for injection may be used. After injection, the eye may be treated with a sterilizing agent such as an antibiotic. The eye may also be rinsed to remove excess sterilizing agents.

Furthermore, in certain examples it is desirable to perform non-invasive retinal imaging and functional studies to identify areas of specific ocular cells to be targeted for therapy. In these examples, clinical diagnostic tests are employed to determine the precise location(s) for one or more subretinal injection(s). These tests may include ophthalmoscopy, RPE function, electroretinography (ERG) (particularly the b-wave measurement, c-wave measurement), perimetry, topographical mapping of the layers of the retina and measurement of the thickness of its layers by means of confocal scanning laser ophthalmoscopy (cSLO) and optical coherence tomography (OCT), topographical mapping of cone density via adaptive optics (AO), functional eye exam, etc. In some examples, one or more injections are performed in the same eye in order to target different areas of retained bipolar cells.

The composition may be delivered in a volume of from about 0.1 μL to about 1 mL, including all numbers within the range, depending on the size of the area to be treated, the route of administration, and the desired effect of the method. In one example, the volume is about 50 μL. In another example, the volume is about 70 μL. In one example, the volume is about 100 μL. In another example, the volume is about 125 μL. In another example, the volume is about 150 μL. In another example, the volume is about 175 μL. In yet another example, the volume is about 200 μL. In another example, the volume is about 250 μL. In another example, the volume is about 300 μL. In another example, the volume is about 450 μL. In another example, the volume is about 500 μL. In another example, the volume is about 600 μL. In another example, the volume is about 750 μL. In another example, the volume is about 850 μL. In another example, the volume is about 1000 μL.

For example, the dose may be between about 100 ng/eye to about 10 mg/eye (e.g., about 100 ng/eye, about 150 ng/eye, about 200 ng/eye, about 250 ng/eye, about 300 ng/eye, about 400 ng/eye, about 500 ng/eye, about 600 ng/eye, about 700 ng/eye, about 800 ng/eye, about 900 ng/eye, about 1 μg/eye, about 2 μg/eye, about 3 μg/eye, about 5 μg/eye, about 10 μg/eye, about 15 μg/eye, about 20 μg/eye, about 25 μg/eye, about 30 μg/eye, about 35 μg/eye, about 40 μg/eye, about 50 μg/eye, about 60 μg/eye, about 70 μg/eye, about 80 μg/eye, about 90 μg/eye, about 100 μg/eye, about 120 μg/eye, about 150 μg/eye, about 175 μg/eye, about 200 μg/eye, about 250 μg/eye, about 300 μg/eye, about 350 μg/eye, about 400 μg/eye, about 500 μg/eye, about 750 μg/eye, about 1 mg/eye, about 1.5 mg/eye, about 2 mg/eye, about 2.5 mg/eye, about 3 mg/eye, about 3.5 mg/eye, about 4 mg/eye, about 4.5 mg/eye, about 5 mg/eye, or any ranges within these).

Still other dosages and administration volumes in these ranges may be selected by the attending physician, taking into account the physical state of the subject, preferably human, being treated, the age of the subject, the particular ocular disorder and the degree to which the disorder, if progressive, has developed. For extra-ocular delivery, e.g., oral delivery and/or intravenous delivery, the dosage may be increased according to the scale-up from the retina.

### Methods of Treatment/Prophylaxis

Described herein are various methods of preventing, treating, arresting progression of or ameliorating the ocular disorders and retinal changes associated therewith. Any of these methods may include identifying the patient that may benefit from one or more of these therapies and/or identifying which one or more of the therapies described herein may be most beneficial to a particular patient. The assays described herein, which may produce a score or characterization of the patient based on one or more biomarkers, as described herein, may be used to identify the patient that may benefit from one or more of these therapies and/or which therapies should be applied. Any of these methods may include determining the dose to be delivered, the delivery route and/or the schedule for delivering one or more doses. The assays described herein, scoring and/or characterizing based on one or more biomarkers, may be used to determine the dose to be delivered, the delivery route and/or the schedule for delivering one or more doses.

Generally, the methods include administering to a mammalian subject in need thereof, an effective amount of any of the compositions described herein. For example, treatment of age-related macular degeneration may include the localized delivery of a therapeutic composition as described herein to the patient's retina. The cells that will be the treatment target in these diseases may include photoreceptor cells in the retina or the cells of the RPE underlying the neurosensory retina or the cells of the choriocapillaris or the Bruch's membrane. In a certain aspect, the disclosure provides a method of treating a subject having age-related macular degeneration (AMD), comprising the step of administering to the subject any of the compositions described herein.

In a particular example or method of preventing, arresting progression of or ameliorating vision loss associated with an ocular disorder in the subject is provided. Vision loss associated with an ocular disorder refers to any decrease in peripheral vision, central (reading) vision, night vision, day vision, loss of color perception, loss of contrast sensitivity, or reduction in visual acuity. The methods and compositions described herein may be directed to increasing photoreceptor function. As used herein, “increase photoreceptor function” means to improve the function of the photoreceptors or increase the number or percentage of functional photoreceptors as compared to a diseased eye (having the same ocular disease), the same eye at an earlier time point, a non-treated portion of the same eye, or the contralateral eye of the same patient. Photoreceptor function may be assessed using a functional study, e.g., ERG or perimetry, which are conventional in the art.

For each of the described methods, the treatment may be used to prevent the occurrence of retinal damage or to rescue eyes having mild or advanced disease. As used herein, the term “rescue” means to prevent progression of the disease to total blindness, prevent spread of damage to uninjured ocular cells, improve damage in injured ocular cells, or to provide enhanced vision. In one example, the composition is administered before the disease becomes symptomatic or prior to photoreceptor loss. “Symptomatic” means the onset of any of the various retinal changes described above or vision loss. In another example, the composition is administered after disease becomes symptomatic. In yet another example, the composition is administered after initiation of photoreceptor loss. In another example, the composition is administered after outer nuclear layer (ONL) degeneration begins. In some examples, it is desirable that the composition is administered while bipolar cell circuitry to ganglion cells and the optic nerve remains intact. In another example, the composition is administered after initiation of photoreceptor loss. In yet another example, the composition is administered when less than 90% of the photoreceptors are functioning or remaining, as compared to a non-diseased eye. In another example, the composition is administered when less than 80% of the photoreceptors are functioning or remaining. In another example, the composition is administered when less than 70% of the photoreceptors are functioning or remaining. In another example, the composition is administered when less than 60% of the photoreceptors are functioning or remaining. In another example, the composition is administered when less than 50% of the photoreceptors are functioning or remaining. In another example, the composition is administered when less than 40% of the photoreceptors are functioning or remaining. In another example, the composition is administered when less than 30% of the photoreceptors are functioning or remaining. In another example, the composition is administered when less than 20% of the photoreceptors are functioning or remaining. In another example, the composition is administered when less than 10% of the photoreceptors are functioning or remaining. In one example, the composition is administered only to one or more regions of the eye. In another example, the composition is administered to the entire eye. In another example, the method includes performing functional and imaging studies to determine the efficacy of the treatment. These studies include ERG and in vivo retinal imaging, as described in the examples below. In addition, visual field studies, perimetry and microperimetry, pupillometry, mobility testing, visual acuity, contrast sensitivity, color vision testing may be performed.

In yet another example, any of the methods described herein may be performed in combination with another, or secondary, therapy. The therapy may be any now known, or as yet unknown, therapy which helps prevent, arrest or ameliorate any of the described retinal changes and/or vision loss.

In some examples, methods of treatment described herein may incorporate identification of a patient based on one or more diagnostic techniques. In particular, identification of one or more genetic aberrations relating to dysfunction of one or more components of a lipid transport and processing mechanism may support the selection of one or more of the therapeutic peptides described herein. In a particular example, an abnormality of the genotype of one or more components of a lipid processing mechanism may result in the observed dysregulation of lipid processing relating to the pathogenesis of AMD. Based on the identified abnormality, one or more of the therapeutic peptides may be administered to support the lipid processing mechanism or cholesterol homeostasis.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a subset of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data are provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

What is claimed is:

1. A method of treating intermediate age-related macular degeneration (AMD), the method comprising administering to a subject an amphipathic ApoA1 mimetic polypeptide that comprises a helical coil having a hydrophobic moment, μH, of 0.65 or greater in an amount sufficient to reduce an amount of drusen in the subject, thereby treating the intermediate AMD.

2. The method of claim 1, wherein the polypeptide has ATP binding cassette transporter binding activity.

3. The method of claim 1, wherein the polypeptide comprises a peptide sequence having at least 80% identity with SEQ ID NO: 87.

4. The method of claim 3, wherein, for the polypeptide, any peptide residues that differ from the sequence of SEQ ID NO.: 87 in positions 2-7, 9 and 11-17 are conservative substitutions or conservative hydrophobicity substitutions having a hydrophobicity value that is within 0.25 of the hydrophobicity value of a peptide residue in a corresponding position of SEQ ID NO.: 87, as calculated using the method of Fauchere and Pliska, and wherein any differing peptide residues in positions 1, 8, 10 and 18 are any amino acid.

5. The method of claim 3, wherein, for the polypeptide, the polypeptide sequence is one of SEQ. ID. NO.: 87, 152, 153, 154, 155, 156, 157, 158, 159, 160, 161, 162, 163, 164, 165, 166, 167, 168, 169, 170, 171, 172, 173, 174, 175, 176, 177 or 178.

6. The method of claim 3, wherein, for the polypeptide, the polypeptide sequence is one of SEQ. ID. NO.: 87, 152, 160, 161, 163 or 172.

7. The method of claim 3, wherein, for the polypeptide, the polypeptide sequence has at least 94% identity with SEQ ID NO: 87.

8. The method of claim 3, wherein, for the polypeptide, the polypeptide sequence is SEQ ID NO: 87.

9. The method of claim 3, wherein, for the polypeptide, the polypeptide sequence is SEQ ID NO.: 35 or 36, wherein X may be any amino acid.

10. The method of claim 1, wherein administering comprises intraocular injection.

11. The method of claim 1, wherein administering comprises intravascular (IV) injection.

12. The method of claim 1, wherein administering comprises subcutaneous (SC) injection.

13. The method of claim 1, wherein the patient is 40 years old or older.

14. A method of treating intermediate age-related macular degeneration (AMD), the method comprising administering to a subject an amphipathic ApoA1 mimetic polypeptide that comprises a helical coil having a hydrophobic moment, μH, of 0.65 or greater, wherein the polypeptide has ATP binding cassette transporter membrane stabilization and agonist activity, and wherein the polypeptide has transporter protein binding activity, further wherein the amphipathic ApoA1 mimetic polypeptide is administered in an amount sufficient to reduce an amount of drusen in the subject, thereby treating the intermediate AMD.

15. The method of claim 14, wherein the polypeptide comprises a peptide sequence having at least 80% identity with SEQ ID NO: 87.

16. The method of claim 15, wherein, for the polypeptide, any peptide residues that differ from the sequence of SEQ ID NO: 87 in positions 2-7, 9 and 11-17 are conservative substitutions or conservative hydrophobicity substitutions having a hydrophobicity value that is within 0.25 of the hydrophobicity value of a peptide residue in a corresponding position of SEQ ID NO: 87, as calculated using the method of Fauchere and Pliska, and wherein any differing peptide residues in positions 1, 8, 10 and 18 are any amino acid.

17. The method of claim 15, wherein, for the polypeptide, the polypeptide sequence is one of SEQ. ID. NO.: 87, 152, 153, 154, 155, 156, 157, 158, 159, 160, 161, 162, 163, 164, 165, 166, 167, 168, 169, 170, 171, 172, 173, 174, 175, 176, 177 or 178.

18. The method of claim 15, wherein, for the polypeptide, the polypeptide sequence has at least 94% identity with SEQ ID NO: 87.

19. The method of claim 15, wherein, for the polypeptide, the polypeptide sequence is SEQ ID NO.: 35 or 36, wherein X may be any amino acid.

20. A method of treating intermediate age-related macular degeneration (AMD), the method comprising administering to a subject an amphipathic ApoA1 mimetic polypeptide that comprises a helical coil having a hydrophobic moment, μH, of 0.65 or greater in an amount sufficient to reduce an amount of drusen in the subject, thereby treating the intermediate AMD, further wherein the amphipathic ApoA1 mimetic polypeptide comprises a peptide sequence having at least 80% identity with SEQ ID NO: 87.


SEQUENCE LISTING (178 sequences)

SEQ ID NO: 1 - protein, 18 aa, synthetic construct
DWFKAFYDKVAEKFKEAF

SEQ ID NO: 2 - protein, 28 aa, synthetic construct
LRKLRKRLLRDWLKAFYDKVAEKLKEAF

SEQ ID NO: 3 - protein, 26 aa, synthetic construct
EVRSKLEEWFAAFREFAEEFLARLKS

SEQ ID NO: 4 - protein, 37 aa, synthetic construct
DWLKAFYDKVAEKLKEAFPDWAKAAYDKAAEKAKEAA

SEQ ID NO: 5 - protein, 22 aa, synthetic construct
PLRAELQEGARQKLHELQEKLS

SEQ ID NO: 6 - protein, 44 aa, synthetic construct
QDEPPQSPWDRVKDLATVYVDVLKDSGRDYVSQFEGSALGKQLN

SEQ ID NO: 7 - protein, 34 aa, synthetic construct
EELRVRLASHLRKLRKRLLRDADDLQKRLAVYQA

SEQ ID NO: 8 - protein, 11 aa, synthetic construct
HLRKLRKRLLR

SEQ ID NO: 9 - protein, 50 aa, synthetic construct
EELRVRLASHLRKLRKRLLRDADDLQKRLAVYQAGAREGAERGLSAIRER

SEQ ID NO: 10 - protein, 22 aa, synthetic construct
RDADDLQKRLAVYQAGAREGAE

SEQ ID NO: 11 - protein, 22 aa, synthetic construct
DADDLQKRLAVYQAGAREGAER

SEQ ID NO: 12 - protein, 22 aa, synthetic construct
QSTEELRVRLASHLRKLRKRLL

SEQ ID NO: 13 - protein, 76 aa, synthetic construct
TRARLSKELQAAQARLGADMEDVRGRLVQYRGEVQAMLGQSTEELRVRLASHLRKLRKRLLRDADDLQKRLAVYQA

SEQ ID NO: 14 - protein, 22 aa, synthetic construct
EQAQQIRLQAEAFQARLKSWFE

SEQ ID NO: 15 - protein, 10 aa, synthetic construct
LVGRQLEEFL

SEQ ID NO: 16 - protein, 18 aa, synthetic construct
SLVSQYFQTVTDYGKDLM

SEQ ID NO: 17 - protein, 45 aa, synthetic construct
PLRAELQEGARQKLHELQEKLSPPLRAELQEGARQKLHELQEKLS

SEQ ID NO: 18 - protein, 35 aa, synthetic construct
HLRKLRKRLLRPPEQAQQIRLQAEAFQARLKSWFE

SEQ ID NO: 19 - protein, 22 aa, synthetic construct
NMDEINKRLHAYEEGVKKAADE

SEQ ID NO: 20 - protein, 22 aa, synthetic construct
DGDKLCDRLAEYCARAKEAFDR

SEQ ID NO: 21 - protein, 29 aa, synthetic construct
HLRKLRKRLLRDWFKAFYDKVAEKFKEAF

SEQ ID NO: 22 - protein, 41 aa, synthetic construct
DWFKAFYDKVAEKFKEAFPEQAQQIRLQAEAFQARLKSWFE

SEQ ID NO: 23 - protein, 18 aa, synthetic construct
DWFKAFYDKVAENFKEAF

SEQ ID NO: 24 - protein, 18 aa, synthetic construct
FEHWISGIYSECKHLIQE

SEQ ID NO: 25 - protein, 29 aa, synthetic construct
HLRKLRKRLLRFEHWISGIYSECKHLIQE

SEQ ID NO: 26 - protein, 18 aa, synthetic construct
GFGDWIDSIEAFSRSLHS

SEQ ID NO: 27 - protein, 29 aa, synthetic construct
HLRKLRKRLLRSLVSQYFQTVTDYGKDLM

SEQ ID NO: 28 - protein, 21 aa, synthetic construct
HLRKLRKRLLRLVGRQLEEFL

SEQ ID NO: 29 - protein, 33 aa, synthetic construct
HLRKLRKRLLRDGDKLCDRLAEYCARAKEAFDR

SEQ ID NO: 30 - protein, 45 aa, synthetic construct
EHLCELIERACRKLVELQEKLSKWLIAELGEGWRQWLHSLKEFLS

SEQ ID NO: 31 - protein, 18 aa, synthetic construct
MKIEHKWFFEETKIEMGK

SEQ ID NO: 32 - protein, 22 aa, synthetic construct
NMDENNKLKAAYVHGEKWEADT

SEQ ID NO: 33 - protein, 37 aa, synthetic construct
DWLKAFYDKVAEKLKEAFPDWLKAFYDKVAEKLKEAF

SEQ ID NO: 34 - protein, 18 aa, synthetic construct
GIKKFLGSIWKFIKAFVG

SEQ ID NO: 35 - protein, 18 aa, synthetic construct
  VARIANT 1: note = X can be any amino acid
  VARIANT 8: note = X can be any amino acid
  VARIANT 10: note = X can be any amino acid
  VARIANT 18: note = X can be any amino acid
XAWERFRXLXKELADYFX

SEQ ID NO: 36 - protein, 16 aa, synthetic construct
  VARIANT 7: note = X can be any amino acid
  VARIANT 9: note = X can be any amino acid
AWERFRXLXKELADYF

SEQ ID NO: 37 - protein, 19 aa, synthetic construct
  VARIANT 6: note = X can be any amino acid
  VARIANT 13: note = X can be any amino acid
  VARIANT 16: note = X can be any amino acid
ADALEXALKELKXFIXEWT

SEQ ID NO: 38 - protein, 15 aa, synthetic construct
  VARIANT 6: note = X can be any amino acid
  VARIANT 13: note = X can be any amino acid
ADALEXALKELKXFI

SEQ ID NO: 39 - protein, 22 aa, synthetic construct
  VARIANT 2..3: note = X can be any amino acid
  VARIANT 9: note = X can be any amino acid
  VARIANT 16: note = X can be any amino acid
  VARIANT 19: note = X can be any amino acid
RXXADALEXALKELKXFIXEWT

SEQ ID NO: 40 - protein, 33 aa, synthetic construct
HLRKLRKRLLRDGDKLQDRLAEYQARAKEAFDR

SEQ ID NO: 41 - protein, 33 aa, synthetic construct
HLRRLRRRLLRDGDKLQDRLAEYQARAKEAFDR

SEQ ID NO: 42 - protein, 33 aa, synthetic construct
ALRLDRLDLKRKGLALQDRKYEAQARDKEHRRF

SEQ ID NO: 43 - protein, 33 aa, synthetic construct
HLRKLRKRLLRDADDLQKRLAVYQAGAREGAER

SEQ ID NO: 44 - protein, 28 aa, synthetic construct
LASHLRKLRKRLLRDADDLQKRLAVYQA

SEQ ID NO: 45 - protein, 25 aa, synthetic construct
HLRKLRKRLLRDADDLQKRLAVYQA

SEQ ID NO: 46 - protein, 36 aa, synthetic construct
LASHLRKLRKRLLRDADDLQKRLAVYQAGAREGAER

SEQ ID NO: 47 - protein, 36 aa, synthetic construct
LARHLRKLRKRLARDADDLQKRLAVYQAGAREGAER

SEQ ID NO: 48 - protein, 36 aa, synthetic construct
LASHLRKLRKRLLRDGDKLQDRLAEYQARAKEAFDR

SEQ ID NO: 49 - protein, 36 aa, synthetic construct
LARHLRKLRKRLARDGDKLQDRLAEYQARAKEAFDR

SEQ ID NO: 50 - protein, 33 aa, synthetic construct
HLRKLRKRLLRDADKLQDRLAEYQARAKEAFDR

SEQ ID NO: 51 - protein, 33 aa, synthetic construct
HLRKLRKRLLRDGDDLQDRLAEYQARAKEAFDR

SEQ ID NO: 52 - protein, 33 aa, synthetic construct
HLRKLRKRLLRDGDKLQKRLAEYQARAKEAFDR

SEQ ID NO: 53 - protein, 33 aa, synthetic construct
HLRKLRKRLLRDGDKLQDRLAVYQARAKEAFDR

SEQ ID NO: 54 - protein, 33 aa, synthetic construct
HLRKLRKRLLRDGDKLQDRLAEYQAGAKEAFDR

SEQ ID NO: 55 - protein, 33 aa, synthetic construct
HLRKLRKRLLRDGDKLQDRLAEYQARAREAFDR

SEQ ID NO: 56 - protein, 33 aa, synthetic construct
HLRKLRKRLLRDGDKLQDRLAEYQARAKEGFDR

SEQ ID NO: 57 - protein, 33 aa, synthetic construct
HLRKLRKRLLRDGDKLQDRLAEYQARAKEAADR

SEQ ID NO: 58 - protein, 33 aa, synthetic construct
HLRKLRKRLLRDGDKLQDRLAEYQARAKEAFER

SEQ ID NO: 59 - protein, 33 aa, synthetic construct
HLRKLVKRLFRDGDKLQDRLAEYQARAKEAFDR

SEQ ID NO: 60 - protein, 33 aa, synthetic construct
HLRKLRKRLFRDGDKLQDRLAEYQARAKEAFDR

SEQ ID NO: 61 - protein, 19 aa, synthetic construct
HLRKLRKRLLRDGDKLQDR

SEQ ID NO: 62 - protein, 33 aa, synthetic construct
HLRKLRKRLLRDGDKLYDRLAEYYARAKEAFDR

SEQ ID NO: 63 - protein, 33 aa, synthetic construct
HLRKLRKRLLRDADDLQDRLAEYQARAKEAFDR

SEQ ID NO: 64 - protein, 33 aa, synthetic construct
HLRKLRKRLLRDADDLQKRLAEYQARAKEAFDR

SEQ ID NO: 65 - protein, 32 aa, synthetic construct
LASHLRKLRKRLLRSLVSQYFQTVTDYGKDLM

SEQ ID NO: 66 - protein, 32 aa, synthetic construct
LARHLRKLRKRLARSLVSQYFQTVTDYGKDLM

SEQ ID NO: 67 - protein, 29 aa, synthetic construct
HYKRLRKRQLMDSVLQYFSTDTLLGVRLK

SEQ ID NO: 68 - protein, 57 aa, synthetic construct
MEDVRGRLVQYRGEVQAMLGQSTEELRVRLASHLRKLRKRLLRDADDLQKRLAVYQA

SEQ ID NO: 69 - protein, 54 aa, synthetic construct
VRGRLVQYRGEVQAMLGQSTEELRVRLASHLRKLRKRLLRDADDLQKRLAVYQA

SEQ ID NO: 70 - protein, 50 aa, synthetic construct
LVQYRGEVQAMLGQSTEELRVRLASHLRKLRKRLLRDADDLQKRLAVYQA

SEQ ID NO: 71 - protein, 42 aa, synthetic construct
LVQYRGEVQAMLGQSTEELRVRLASHLRKLRKRLLRDADDLQ

SEQ ID NO: 72 - protein, 14 aa, synthetic construct
LASHLRKLRKRLLR

SEQ ID NO: 73 - protein, 18 aa, synthetic construct
LASHLRKLRKRLLRDADD

SEQ ID NO: 74 - protein, 20 aa, synthetic construct
EELRVRLASHLRKLRKRLLR

SEQ ID NO: 75 - protein, 24 aa, synthetic construct
EELRVRLASHLRKLRKRLLRDADD

SEQ ID NO: 76 - protein, 16 aa, synthetic construct
HLRKLRKRLLRDADDL

SEQ ID NO: 77 - protein, 33 aa, synthetic construct
HLRKLRKRLLRDGDKLVDRLAEYVARAKEAFDR

SEQ ID NO: 78 - protein, 33 aa, synthetic construct
HLRKLRKRLLRDGDKLSDRLAEYSARAKEAFDR

SEQ ID NO: 79 - protein, 33 aa, synthetic construct
HLRKLRKRLLRDGDKLVDRLAEYSARAKEAFDR

SEQ ID NO: 80 - protein, 33 aa, synthetic construct
HLRKLRKRLLRDGDKLSDRLAEYVARAKEAFDR

SEQ ID NO: 81 - protein, 18 aa, synthetic construct
KWFKAFYKDVAEDFKEAF

SEQ ID NO: 82 - protein, 18 aa, synthetic construct
DKFKAFYDKVAEWFKEAF

SEQ ID NO: 83 - protein, 18 aa, synthetic construct
AKAWEKFVKEFAKYFDDF

SEQ ID NO: 84 - protein, 18 aa, synthetic construct
REFRALFKELADYFRDAW

SEQ ID NO: 85 - protein, 18 aa, synthetic construct
WREFRALFKELADYFRDA

SEQ ID NO: 86 - protein, 18 aa, synthetic construct
WERFRALFKELADYFRDA

SEQ ID NO: 87 - protein, 18 aa, synthetic construct
DAWERFRALFKELADYFR

SEQ ID NO: 88 - protein, 18 aa, synthetic construct
FDDYLSRFYSQLRRAFHE

SEQ ID NO: 89 - protein, 18 aa, synthetic construct
FRDYASRFYSQLRDAFHE

SEQ ID NO: 90 - protein, 18 aa, synthetic construct
HEFDDSLSRFYSQLRRAF

SEQ ID NO: 91 - protein, 18 aa, synthetic construct
FEHWISGIYSELKHLIQE

SEQ ID NO: 92 - protein, 18 aa, synthetic construct
FEHWISGIYSEAKHLIQE

SEQ ID NO: 93 - protein, 22 aa, synthetic construct
LPVLESFKVSFLSALEEYTKKL

SEQ ID NO: 94 - protein, 22 aa, synthetic construct
EKEIRSVLSGATKHFEKILDKL

SEQ ID NO: 95 - protein, 22 aa, synthetic construct
EREIRSVLSGATRHFEDILDKL

SEQ ID NO: 96 - protein, 22 aa, synthetic construct
AKSLSRVLSGATKHFRTILDEL

SEQ ID NO: 97 - protein, 22 aa, synthetic construct
ARMEEMGSRTRDRLDEVKEQVA

SEQ ID NO: 98 - protein, 22 aa, synthetic construct
KRMERMLDETRDTLTRFSEQWT

SEQ ID NO: 99 - protein, 22 aa, synthetic construct
RRMKDYLKKFQETLKREFRDWE

SEQ ID NO: 100 - protein, 22 aa, synthetic construct
RRGDKALSDTKDELKRFIEEWT

SEQ ID NO: 101 - protein, 22 aa, synthetic construct
RSGADALESALKELKRFIREWT

SEQ ID NO: 102 - protein, 22 aa, synthetic construct
TVGSLAGQPLQERAQAWGERLR

SEQ ID NO: 103 - protein, 22 aa, synthetic construct
TVGRLAKDHLHELTRGWGKFER

SEQ ID NO: 104 - protein, 22 aa, synthetic construct
TVRGSADALLEHLTRAWDGFNR

SEQ ID NO: 105 - protein, 22 aa, synthetic construct
DALSRGVEGLARAWDTFNRHLT

SEQ ID NO: 106 - protein, 22 aa, synthetic construct
EALSKGVEGLAKAWETFNKHLT

SEQ ID NO: 107 - protein, 18 aa, synthetic construct
NHINDVQRDVKKTIERLF

SEQ ID NO: 108 - protein, 18 aa, synthetic construct
DHINKVQRKVTHEIERLF

SEQ ID NO: 109 - protein, 28 aa, synthetic construct
HLRKLRKRLLRSLVSQYFQTVTDYGKDL

SEQ ID NO: 110 - protein, 28 aa, synthetic construct
HLRKLRKRLLRSLVSQYRQTVTDYGKDL

SEQ ID NO: 111 - protein, 28 aa, synthetic construct
HLRKLVKRLLRSLVSQYRQTVTDYGEDL

SEQ ID NO: 112 - protein, 57 aa, synthetic construct
YKDFGERVERALGRQLEMLGQSTEELRVRLASHLRKLRKRLLRDADDLQKRLAVYQA

SEQ ID NO: 113 - protein, 57 aa, synthetic construct
WKDFGERVERALREQFEDLGQSTEELRVRLASHLRKLRKRLLRDADDLQKRLAVYQA

SEQ ID NO: 114 - protein, 57 aa, synthetic construct
LKDFGERVARALREQFEDLGQSTEELRVRLASHLRKLRKRLLRDADDLQKRLAVYQA

SEQ ID NO: 115 - protein, 57 aa, synthetic construct
DHINKVQRKVTHEIERLFLGQSTEELRVRLASHLRKLRKRLLRDADDLQKRLAVYQA

SEQ ID NO: 116 - protein, 57 aa, synthetic construct
DAWERFRALFKELADYFRLGQSTEELRVRLASHLRKLRKRLLRDADDLQKRLAVYQA

SEQ ID NO: 117 - protein, 61 aa, synthetic construct
RSGADALESALKELKRFIREWTLGQSTEELRVRLASHLRKLRKRLLRDADDLQKRLAVYQA

SEQ ID NO: 118 - protein, 76 aa, synthetic construct
LVARLLKELTAAMARLGRDAEDIRGNLFQYFDEVWAMLGQSTEELRVRLASHLRKLRKRLLRDADDLQKRLAVYQA

SEQ ID NO: 119 - protein, 76 aa, synthetic construct
DVKRLFKEFTAAMARFARKAEDIRGNFDQYFDEVFAMLGQSTEELRVRLASHLRKLRKRLLRDADDLQKRLAVYQA

SEQ ID NO: 120 - protein, 76 aa, synthetic construct
DVKRLLKELTEAMAELARKAEDIRGNLDQYFRKVFAMLGQSTEELRVRLASHLRKLRKRLLRDADDLQKRLAVYQA

SEQ ID NO: 121 - protein, 22 aa, synthetic construct
ASGADALESALKELKRFIREWT

SEQ ID NO: 122 - protein, 22 aa, synthetic construct
RAGADALESALKELKRFIREWT

SEQ ID NO: 123 - protein, 22 aa, synthetic construct
RSAADALESALKELKRFIREWT

SEQ ID NO: 124 - protein, 22 aa, synthetic construct
RSGPDALESALKELKRFIREWT

SEQ ID NO: 125 - protein, 22 aa, synthetic construct
RSGAAALESALKELKRFIREWT

SEQ ID NO: 126 - protein, 22 aa, synthetic construct
RSGADPLESALKELKRFIREWT

SEQ ID NO: 127 - protein, 22 aa, synthetic construct
RSGADAAESAAKELKRFIREWT

SEQ ID NO: 128 - protein, 22 aa, synthetic construct
RSGADALASALKELKRFIREWT

SEQ ID NO: 129 - protein, 22 aa, synthetic construct
RSGADALEAALKELKRFIREWT

SEQ ID NO: 130 - protein, 22 aa, synthetic construct
RSGADALESPLKELKRFIREWT

SEQ ID NO: 131 - protein, 22 aa, synthetic construct
RSGADALESAAKELKRFIREWT

SEQ ID NO: 132 - protein, 22 aa, synthetic construct
RSGADALESALAELKRFIREWT

SEQ ID NO: 133 - protein, 22 aa, synthetic construct
RSGADALESALKALKRFIREWT

SEQ ID NO: 134 - protein, 22 aa, synthetic construct
RSGADALESALKEAKRFIREWT

SEQ ID NO: 135 - protein, 22 aa, synthetic construct
RSGADALESALKELARFIREWT

SEQ ID NO: 136 - protein, 22 aa, synthetic construct
RSGADALESALKELKAFIREWT

SEQ ID NO: 137 - protein, 22 aa, synthetic construct
RSGADALESALKELKRAIREWT

SEQ ID NO: 138 - protein, 22 aa, synthetic construct
RSGADALESALKELKRFAREWT

SEQ ID NO: 139 - protein, 22 aa, synthetic construct
RSGADALESALKELKRFIAEWT

SEQ ID NO: 140 - protein, 22 aa, synthetic construct
RSGADALESALKELKRFIRAWT

SEQ ID NO: 141 - protein, 22 aa, synthetic construct
RSGADALESALKELKRFIREAT

SEQ ID NO: 142 - protein, 22 aa, synthetic construct
RSGADALESALKELKRFIREWA

SEQ ID NO: 143 - protein, 22 aa, synthetic construct
RSGGDALESALKELKRFIREWT

SEQ ID NO: 144 - protein, 22 aa, synthetic construct
RSGADGLESALKELKRFIREWT

SEQ ID NO: 145 - protein, 22 aa, synthetic construct
RSGADALESGLKELKRFIREWT

SEQ ID NO: 146 - protein, 22 aa, synthetic construct
AAAADALESALKELKRFIREWT

SEQ ID NO: 147 - protein, 22 aa, synthetic construct
RSGADAAAAALKELKRFIREWT

SEQ ID NO: 148 - protein, 22 aa, synthetic construct
RSGADALESAAAALKRFIREWT

SEQ ID NO: 149 - protein, 22 aa, synthetic construct
RSGADALESALKEAAAFIREWT

SEQ ID NO: 150 - protein, 22 aa, synthetic construct
RSGADALESALKELKRAAAEWT

SEQ ID NO: 151 - protein, 22 aa, synthetic construct
RSGADALESALKELKRFIRAAA

SEQ ID NO: 152 - protein, 18 aa, synthetic construct
AAWERFRALFKELADYFR

SEQ ID NO: 153 - protein, 18 aa, synthetic construct
DPWERFRALFKELADYFR

SEQ ID NO: 154 - protein, 18 aa, synthetic construct
DGWERFRALFKELADYFR

SEQ ID NO: 155 - protein, 18 aa, synthetic construct
DAAERFRALFKELADYFR

SEQ ID NO: 156 - protein, 18 aa, synthetic construct
DAWARFRALFKELADYFR

SEQ ID NO: 157 - protein, 18 aa, synthetic construct
DAWEAFRALFKELADYFR

SEQ ID NO: 158 - protein, 18 aa, synthetic construct
DAWERARALFKELADYFR

SEQ ID NO: 159 - protein, 18 aa, synthetic construct
DAWERFAALFKELADYFR

SEQ ID NO: 160 - protein, 18 aa, synthetic construct
DAWERFRPLFKELADYFR

SEQ ID NO: 161 - protein, 18 aa, synthetic construct
DAWERFRGLFKELADYFR

SEQ ID NO: 162 - protein, 18 aa, synthetic construct
DAWERFRAAFKELADYFR

SEQ ID NO: 163 - protein, 18 aa, synthetic construct
DAWERFRALAKELADYFR

SEQ ID NO: 164 - protein, 18 aa, synthetic construct
DAWERFRALFAELADYFR

SEQ ID NO: 165 - protein, 18 aa, synthetic construct
DAWERFRALFKALADYFR

SEQ ID NO: 166 - protein, 18 aa, synthetic construct
DAWERFRALFKEAADYFR

SEQ ID NO: 167 - protein, 18 aa, synthetic construct
DAWERFRALFKELPDYFR

SEQ ID NO: 168 - protein, 18 aa, synthetic construct
DAWERFRALFKELGDYFR

SEQ ID NO: 169 - protein, 18 aa, synthetic construct
DAWERFRALFKELAAYFR

SEQ ID NO: 170 - protein, 18 aa, synthetic construct
DAWERFRALFKELADAFR

SEQ ID NO: 171 - protein, 18 aa, synthetic construct
DAWERFRALFKELADYAR

SEQ ID NO: 172 - protein, 18 aa, synthetic construct
DAWERFRALFKELADYFA

SEQ ID NO: 173 - protein, 18 aa, synthetic construct
AAAERFRALFKELADYFR

SEQ ID NO: 174 - protein, 18 aa, synthetic construct
DAWAAARALFKELADYFR

SEQ ID NO: 175 - protein, 18 aa, synthetic construct
DAWERFAAAFKELADYFR

SEQ ID NO: 176 - protein, 18 aa, synthetic construct
DAWERFRALAAALADYFR

SEQ ID NO: 177 - protein, 18 aa, synthetic construct
DAWERFRALFKEAAAYFR

SEQ ID NO: 178 - protein, 18 aa, synthetic construct
DAWERFRALFKELADAAA